SEQUENCE LISTING (237 sequences)

SEQ ID NO: 1 - DNA, 50 bp, synthetic construct
  source 1..50: mol_type = other DNA; note = A 50-nucleotide sequence representing the 5' junction region of soybean genomic DNA and the integrated transgenic expression cassette.
tacatatataataaaaccttgaattagtcagcatcatcacaccaaaagtt

SEQ ID NO: 2 - DNA, 50 bp, synthetic construct
  source 1..50: mol_type = other DNA; note = A 50-nucleotide sequence representing the 3' junction region of the integrated transgenic expression cassette and the soybean genomic DNA.
ttcccggacatgaagccatttacaaccatagatgtgaggaagaatgtcaa

SEQ ID NO: 3 - DNA, 100 bp, synthetic construct
  source 1..100: mol_type = other DNA; note = A 100-nucleotide sequence representing the 5' junction region of soybean genomic DNA and the integrated transgenic expression cassette.
ccgttgtagtgtctgtttctcttagtacatatataataaaaccttgaattagtcagcatcatcacaccaaaagttaggcccgaatagtttgaaattagaa

SEQ ID NO: 4 - DNA, 100 bp, synthetic construct
  source 1..100: mol_type = other DNA; note = A 100-nucleotide sequence representing the 3' junction region of the integrated transgenic expression cassette and the soybean genomic DNA.
atactcattgctgatccatgtagatttcccggacatgaagccatttacaaccatagatgtgaggaagaatgtcaaaaatggaataaattttataaccact

SEQ ID NO: 5 - DNA, 200 bp, synthetic construct
  source 1..200: mol_type = other DNA; note = A 200-nucleotide sequence representing the 5' junction region of soybean genomic DNA and the integrated transgenic expression cassette.
caaaattgtgataatatattaaatttagcataaaaagaaatgcaaataccccgttgtagtgtctgtttctcttagtacatatataataaaaccttgaattagtcagcatcatcacaccaaaagttaggcccgaatagtttgaaattagaaagctcgcaattgaggtctgtcgaccctgcactaactataacggtcctaag

SEQ ID NO: 6 - DNA, 200 bp, synthetic construct
  source 1..200: mol_type = other DNA; note = A 200-nucleotide sequence representing the 3' junction region of the integrated transgenic expression cassette and the soybean genomic DNA.
attgaaaaaaaattggtaattactctttctttttctccatattgaccatcatactcattgctgatccatgtagatttcccggacatgaagccatttacaaccatagatgtgaggaagaatgtcaaaaatggaataaattttataaccactagcagaaatagaagcactacaatgtataactgcctttcccatgttttatt

SEQ ID NO: 7 - DNA, 1181 bp, synthetic construct
  source 1..1181: mol_type = other DNA; note = A 1,181-nucleotide sequence representing the 5' junction region of soybean genomic DNA and the integrated transgenic expression cassette.
ataataacacaaaagagaatcaaaatgtttattggaaccagctctattcttagtttatttttattttggtgatatatttttaatttgttagaaataaatggtgtaacagttttaattagttttaatggtttgaacagttaattataatgaatgtatgagatgtgtgtactttattctccaaactctaaagtactgactttaaataaaaaatagttatcattgaaaaggtgttcatttttataactgttaacctataacttcttctatagttttgggtacaaaaactgttcacgtacgtacgtattcttataactaactgttaacctataacttgtttttctctcttcatcatattttaatttccgttttttcccctcatttctgtttctttgtatcgtagaaacatagactccgaccaatgccaaaatctttgatcttcattgttcatattctccacatctcaatcgattgttgcagaatcacagaaggagaaagaacataaagaaaagcttgcatttttgcctcaatatcatgttatttttgtttttggaggggagagataactacactaatctcttacgagatcagtgttaatattactacaaaaaagtgataatgatatttacatttttttaagtaaaacatatacatcttctaaaaaaaaagtaaaatatatacatatctctaatttatatttacatacgcatgcaatgttaggtatattcagcaatgcaacactattttcttttttctctataccgttttttctttattattgcatggatagtaggaaaaaataaataattaaaccaaaattaatatataagtcaatatattaaagataaattgttactagcaactttagcataataaaaatgggatatataattttagtgcagtcaaaattgtgataatatattaaatttagcataaaaagaaatgcaaataccccgttgtagtgtctgtttctcttagtacatatataataaaaccttgaattagtcagcatcatcacaccaaaagttaggcccgaatagtttgaaattagaaagctcgcaattgaggtctgtcgaccctgcactaactataacggtcctaaggtagcgacttaggctgagcccgggcaggcctacccataatacccataatagctgtttgccaatcgttcttcttggcgcgcc

SEQ ID NO: 8 - DNA, 1192 bp, synthetic construct
  source 1..1192: mol_type = other DNA; note = A 1,192-nucleotide sequence representing the 3' junction region of the integrated transgenic expression cassette and the soybean genomic DNA.
ctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaaccatagatgtgaggaagaatgtcaaaaatggaataaattttataaccactagcagaaatagaagcactacaatgtataactgcctttcccatgttttatttgtttcgtatgatctaagatgttaagttgacatgtttattaatcttgaagcactattaatttatttctacttatctgtacttttttttattcttaatttaataattttttcctcaatatattttaaaaatatatattaaactaaaattaatataaatgttaaagcatgataaaataaaatgttttgatatatcaaatttaacaaataaaaatacggacacacaaataagataacacatgtgtttttcattttgctaagtatatatattattgatcatatatcttatgattcatgatttgattcacgattccatacataggctttccgattcaactaccttgctggtaattatattgtctcacaccaagcatcaaaattatttgcaattacaaattaattgtttgctagttgttactgatgtcaatggtaaggaggcgttccgaggatctccttcgtttacttttgggctccatatcttccaaatgatgagtgatattgtcactattgatcaattttcactttaaaactgaatcattctgattaaaaaattaattcaaattaagtaactgattgaatttaaaattatatggaaaacaaagatatagattatgaatgagttgtcgctttgcttgttgttattgttaagtgaatttgggttcattgatttcaatgccgctggaatcaaatttgtaagtgaatttggagagattcttggataaatgtatcatatatttcatagtagcatgcatctcattctttgatttgggataaattagcaatttttgctattttacaggtccaatatgttcctgaacaattagaatcactaagggtacgtagtcctttgataactgccacatatatggagctcgaagatggactgagtagtagggaatggag

SEQ ID NO: 9 - DNA, 12240 bp, synthetic construct
  source 1..12240: mol_type = other DNA; note = A 12,240-nucleotide sequence corresponding to the transgenic inserted T-DNA of Soybean event GM_CSM63770.
agtcagcatcatcacaccaaaagttaggcccgaatagtttgaaattagaaagctcgcaattgaggtctgtcgaccctgcactaactataacggtcctaaggtagcgacttaggctgagcccgggcaggcctacccataatacccataatagctgtttgccaatcgttcttcttggcgcgccgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtggtaccaccatggagattgtgaacaaccagaaccagtgcgttccttacaactgcttgaacaaccctgagattgagattcttgagggtggtagaatttctgttggcaacactcctattgacatctctttgagtttgactcaattcttgttgagtgagttcgttcctggtgctggtttcgtcttgggtttgattgatttgatttggggtttcgttggtcctagtcaatgggatgctttcttggctcaagttgagcaattgattaaccagaggatcgctgaggctgtgaggaacactgctattcaagagttggagggtatggctagagtttacagaacttacgctactgctttcgctgagtgggagaaggctcctgatgaccctgagttgagggaggctttgagaactcaattcactgctactgagacttacatcagtggtagaatcagtgtcttgaagattcaaactttcgaggttcaattgctttctgtgttcgctcaagctgcaaacttgcacttgtctttgcttagagatgttgtgttctttggtcaaagatggggtttctccactactaccgtgaacaattactacaacgatttgactgagggtatttctacttacactgattacgctgttagatggtacaacactggtttggagagagtttggggtccagattccagagattgggtcagatacaaccagttcagaagggagttgactttgactgtcttggacattgttgctctcttccctaactacgatagtcgtcgttaccctattagaactgtttctcaacttactagggaaatctacactaaccctgttcttgagaacttcgatggtagtttccgtggtagtgctcaagggattgagcgttctattcgttctcctcatcttatggacattcttaactctattactatctacactgatgctcatcgtggttactattactggtctggtcatcaaattatggctagtcctgttggtttcagtggtcctgagttcactttccctctttacggtactatgggcaacgctgcacctcaacagaggatcgttgctcaacttggtcaaggtgtttacaggactctttcttcaaccctttacaggcgtcctttcaacattgggatcaacaaccagcagctttctgttcttgatggaaccgagttcgcttacggaacctcttcaaaccttcctagtgctgtttacaggaagtctggaaccgttgacagtcttgatgagattccaccgcagaacaataacgttccacccaggcaaggcttcagtcataggctttctcatgtttctatgttccgctctggattcagcaactcttcagtttctattatcagggctccaatgttctcgtggattcataggtctgccgagttcaacaacattatcgcttccgatagcattaaccagattccacttgttaagggattccgtgtttggggaggcacctctgttattaccggaccaggcttcaccggaggcgacattcttcgtcgtaacaccttcggagatttcgtttcacttcaagtgaacattaactcaccaatcacccagcgctacaggcttcgcttccgctacgcatcatccagggatgcaagggtgatcgtgcttaccggagcagcctcaaccggagtgggaggccaagtgagcgtgaacatgccacttcagaagacgatggagatcggcgagaaccttacctcaagaacctttcgttacaccgatttcagcaacccattcagctttcgtgcaaacccagacatcatagggatctcagagcagccactgtttggagctggatcaatctcatccggagagctttacatcgacaagatcgagatcatactcgcagatgcaaccttcgaggctgagagcgatctggagcgtgcacagaaggcagtgaacgcactctttacctctaccaaccagctcggactcaagaccaacgtgaccgattaccacatcgaccaagtgagcaacctcgtgacctacctctcagatgagttctgcttggatgagaaacgcgaactcagcgagaaggtgaagcacgcaaagcgtctctcagatgagcgtaacctcctccaggatagcaatttcaaggacatcaatcgtcagccagagcgtggatggggaggctcaaccggaatcaccatccagggaggcgatgatgtgtttaaggagaattacgtgacactctccggaacattcgatgagtgctacccaacatacctctatcagaagatcgacgagtccaagctcaaggcgttcacccgttatcagctccgtggctacatcgaggatagtcaagacctggaaatctacctcatccgctacaatgcaaagcacgagacagtgaatgtaccaggaacaggctccctctggccactctccgcacagtctccaatcggcaagtgcggcgagccaaatcgctgcgcgccacacctggagtggaatcccgacctggactgctcctgccgcgacggcgagaagtgcgcccaccactcccaccactttagcctggacatcgacgtgggctgtacagacctgaatgaggatctgggcgtgtgggtgatctttaagatcaagacacaggacggccacgcccgcctgggcaatctggagtttctggaggagaagcctctggtgggcgaagccctggcccgcgtgaagcgcgccgagaagaaatggcgcgacaaacgcgagaaactggaatgggaaacaaacatcgtgtacaaagaagccaaagaatccgtggacgccctatttgtgaactcccagtatgaccagctacaggccgacacaaacatcgcgatgatccacgctgcggacaagcgcgtgcactccatacgcgaagcctatctacccgaactatccgtgatacccggcgtcaatgccgcgatctttgaagaattggaaggccgcatcttcacagcctttagcctctatgacgcccgaaatgtcatcaagaatggcgactttaacaatgggctatcctgttggaatgtcaaagggcacgtggacgtcgaagagcagaacaatcagcgatccgtcttagtcgtacccgaatgggaagccgaagtctcccaggaagtccgagtctgtcctggtagaggttacatcttgagagtgactgcttacaaggagggttacggtgagggatgcgtgactattcacgagattgagaacaacactgatgagttgaagttcagtaactgcgtggaggaggaaatctaccccaacaacactgtgacttgtaacgattacaccgtgaaccaggaggaatacggaggcgcttacacctccagaaaccgtggatacaatgaggctccctcggtccccgctgattatgcctccgtctatgaggagaagtcctacaccgatggaaggcgcgagaatccctgcgagttcaatcgcggctatcgagactacactccgctacccgttggctatgtcacaaaggaactggaatacttcccggaaacagacaaagtctggatcgaaatcggcgaaacagaagggacgttcatagtcgatagcgtagaacttctccttatggaagaatgacctgcaggtcattcaatttgacccttttgctacattcttgctgtcatagttatttaatttaatttcttaaggtgcatattcttttggtgaatttaagcccccccgaaacatggaaggaggttggctgcaaagttcctgaaggataatcgtttttttcattccctttaatttttgttattcattatttcacattttacctaaaacttcagggagtgagataaagaactctttctctctgaactgtttatgttgtctactagaaaaaaaaatcctgtaagaggtaaggtattcttccaaaacaggattctttttcttatcattttacttcattagtttctaagttccttttcctacattgtataagattctgtcagtacatttatgttgcttatgtaggaagtgggactgagtttcttatgaaggaatttatgagttattgaatatttattgcaagctatattttagaatacagtatactttcttcttatgttattatttttttaagtgtattgtgttgtgttattattataagctttgttaaactctgatcattttcaaattcaaaacaatgtttagttcaaaggcaaagctgagttgtgctgattatttagggataatagctgtttgccaatcgttcttcttggcgcgccacgtcctgcttggcctactaggcccggaccgacggtacctcagcgctgtgcctgttgcgaaaaacccaaataaatacaaagttaatagattttcaacaatgcggggatagctcagttgggagagcgtcagactgaagatctgaaggtcgcgtgttcgatccacgctcaccgcatcttttttttgttctgtttttcccagcgaatgaactatccatacgtcgtcgtttgtatagccatgatccatgaagcgctctaattaccaatcattttttctttcttggaattgatcaagttacaaatataaaattattttacactaaaaactctcttttaaaaatttagatccaattttaattttgttttctaaaagattgtatccaagaagtaaaatttacatggaaaataaaatgcctataaattaaaaaatgttatattttcgtgacctttaaccttaggaacatttgataactattactttgagattgcagtcataggcaacacaaggtcttggacaaacatgttcgtgacaccaaacacccccctcaaaattaagtatattataccaactcatttcatttgcttgttacggttgtgttttatcaatccttcctcgtcttgttttttcctccaaatttgtaatcttaaaattggtgaaacatcacatatttgattctcaatcttagattttctaccttttaggtaatctatattttgaaattgaaattgaaattgaattccgtgtaaaatctataatatgggaacccaattcgaatttaatgttttatgtaaaggccaataatgttccaaatttatttccttattattattgtatattttgtccccaagtacatcatagattggctttcaattccaaatccatcatggtatatttttaggtgcaaactattgtcgtagatttaatcccctagttttgttgattttttaaaatagtactttcaacaattttaaattttatttgattggttataaacttcgggtgtgatttagttgtccaatttggattatattgtcattttcatacataaatttatttatttttgaaaaagttatcttttagtctttgaaattgaaatttttatgaatatgtggatttagtccctcaattctcaaattaaccatcttcacaagagtgagaaacagagatgggagggagaaaaatggaaagataaaaaaagtatttttcttcctccatgttattaggtcacatcaatatttataatattttatattaaatttaatgatttcttttgggagaggtactcttagacctatttttttaacaaaaattattggactcataattttttaattgtcctttaatttcttttatttgtatctagactttataaagtttgagagttttatattttaaggagaaatatattaaaatataaggttagttttcatacactagatagacttatgatcttatacgagtgaatgagttaaactctcaattaaaattatttgaattttttcaaaaattaaaatcatccattttgttaaattaaaatttgaagaaatttataggatgcgtaagaattgatgccttgttaattttcaaatataattatagtaaaaattcttaattagtttactaacaacgatttaaaagtttactttgatgtgttgaacaatatttatcaaaccaaatttgatcaagtggatatactaacaaaagttcaagatcttaaactttaagtgaattaattttgacatatatatatatataaaaaaaaaaagttaggtgttaaaagaaaactgaaaattgttaaaagaaaagaaaaggagggtatagataaaatgtggatagaaagagatgagttgtagatgcaaaaaaatctttagaaacaatatgattggctaactaagccctctcattaccccacaaaatttctcatatctttcactcttcaccactcatctcctcgtttcctcataatattcccccacttccactctctctcacacaatctccaacactactgcccattcctctgttctttctctctactctcaaccacagagcttcaagtacccaccatgaccagcaaccgaaagaacgagaacgagatcatcaacgccctgtccataccggccgtgtcaaaccactccgcccagatgaacctctccaccgacgcgaggatcgaggactccctctgcatcgccgagggcaacaacatcgacccgttcgtgtctgcaagcacggtccagaccggcatcaacatcgcgggccgcatcctgggcgtgctcggcgtgcccttcgcgggtcaaatcgcctctttctactcattcctcgtgggcgagctgtggccgcgcggacgtgacccgtgggaaatcttcctggagcacgttgagcagctcatccggcagcaagtgaccgagaacaccagggacaccgcactggcacggctccagggccttggcaacagcttccgcgcctaccagcagtcgctggaggactggctggagaaccgagacgacgccagaacccgctcagttctgtacacacagtacatcgccctagagctggacttcctcaacgctatgccgctcttcgccatccgtaaccaggaagtaccgcttctgatggtgtacgcacaagcagcgaacctccatctgctcctgctgcgagacgcatctctgttcggcagtgagttcgggctgacgagccaggagatccagcgctactacgagcgccaagtggagaagactcgtgagtacagcgactactgcgcgcgctggtacaacacgggcttgaacaaccttcgcgggacaaacgccgaatcctggcttcgctacaaccagttccgccgcgacctcacgctgggtgtgctggacctggtcgcgctcttcccgtcctacgacacacgggtgtacccaatgaacacgagcgcacagctcacccgtgagatctacacagatcccatcggccgcaccaacgctcccagtggcttcgcaagcacgaattggttcaacaataacgctccttctttctctgccatcgaggccgctgtcatcagaccgccgcacttactcgatttcccggagcagctcactatcttctctgtgttgtcccggtggtcgaacacgcagtacatgaactactgggtgggccacaggctagagagccggaccatccgtggcagtctctcaacctcgacccacggcaacacgaacacgagcatcaaccctgtcactctccagtttacatctagggacgtttacaggacagagtcgttcgctggcattaacattctgttgaccactccggtgaacggcgtcccttgggcccgcttcaactggaggaatcctctgaactcactgcgcggcagccttctctacactatcggctacaccggcgttgggacgcaactcttcgactcggagaccgagctgccgcccgagaccaccgagcggcctaactacgagagttattcacacaggctctccaacatccgcttgatttctgggaacaccttgcgggctccggtgtactcctggacgcaccgcagcgccgacagaactaataccatcagctccgactcgatcacccagatcccgctggtgaaggctcacacgcttcagtcgggcaccacagtcgtcaagggccctggcttcaccggcggcgacatcctgcgtcgcacatctggcggacccttcgccttcagcaacgtgaacttggacttcaatttgtcacagcggtatcgtgccagaatccggtacgccagcactacgaacctgcgaatctatgttactgtggcgggcgagcggatcttcgccgggcaattcgacaagacgatggacgcgggagcacctctgacattccagtcattctcttacgccacgatcaacacggcattcacgtttccggagcgttccagtagcctgaccgtgggcgctgataccttcagtagcgggaacgaggtgtacgttgaccgtttcgagctgatcccggtcaccgccaccttcgaagctgagtcggacctggagcgtgcacagaaggcagtcaacgagctgttcacctctagcaaccagatcggcctcaagaccgacgtcacagactaccacatcgaccaagtgtccaacctggtcgagtgccttagcgacgagttctgcctagacgagaagaaggagctgtcggagaaggtcaaacacgccaagcgtctgagcgatgagcgcaacctgctccaagaccctaacttccgtggcatcaacaggcagcttgaccgtggctggcgcggctcgacggacatcacgatccagggtggcgacgacgtattcaaggagaattacgtgaccttgcttgggacgtttgacgagtgctatcccacctacctctaccagaagattgatgaatcgaaattgaaggcgtacacgagataccagctccgtggctacatcgaggacagccaggacttggagatctacctcatacgctacaacgctaaacatgagaccgtgaacgtccctgggacgggcagtctgtggccactctctgctcctagccctatcggcaagtgcgctcaccactcgcaccacttcagccttgacatcgacgtgggatgtactgacctcaacgaagacctgggcgtctgggttatcttcaagatcaagacccaggacggccacgcccgactcggcaacctggagttcctggaggagaaaccactggtgggcgaggcgctcgcccgcgtgaagcgtgccgagaagaagtggcgggacaagagggagaagctagaatgggagacgaacatcgtgtacaaggaggccaaggaaagcgtcgatgccctgttcgtgaactcacagtacgaccgtctccaggcggacacgaacatcgccatgatccacgcggctgacaagcgcgtccactccatccgcgaggcgtacttaccggagctgtcggtgatcccaggcgtaaacgcggcgatcttcgaggagctagagggacgcatcttcacagcgttcagcctgtacgacgcacgcaacgtcatcaagaacggcgatttcaacaacggactgtcctgctggaacgtgaagggccacgtcgatgtcgaggaacagaacaaccaccgctctgtcctggtggtcccagagtgggaggccgaggtctcccaggaggtccgcgtgtgccctgggcgtggctacatcctccgtgtgacagcctacaaggagggctacggtgagggctgcgtcaccattcacgagatcgagaacaacactgacgaactcaagttctcgaattgcgtggaggaggaggtgtacccgaacaatacggtgacgtgcaacgactacacggcaacccaagaggagtacgagggcacctacaccagtaggaaccgtggctacgacggtgcctacgagtcgaactccagcgtccctgcggactacgccagcgcgtacgaggagaaggcttacaccgacggacgccgggacaacccatgcgagagcaaccgtggctacggcgactacactcctctcccggccggatacgtcacaaaggagctggagtatttcccagagacggacaaggtgtggatcgaaatcggagagacagagggaaccttcatcgtggacagcgtggagctgctcctcatggaggagtgagaaaactagtggtttatttactatatgttattgtatctttcaataaagagatgctccatttgaccaaaaataaaaagagatgctccagatagtgcatcttatctatgttataataatcaagtgtatgcatttccttcccctttatttggagcatgtgtatgtttaaatatttgtaatttgaaccttcaaacttgttctagttaccaaataataagatcattattagtttgtgtgtttatctctattgtctactaattttattttttttttacttttctattctttatttgtattaaatcataagttctaattatttaatcaaaacttcaaatggtggcggttatacgctattccgaatcgtcttttgttgtccgagtctataggctacactcatatcctggttgaaacagactctatagctgtcctgatcaggtcaatcagattagaagattcttttctcaaaattggagtcttttctttcaacgctgtgcagattgacttcgccagcagaacacgcgctgaggttaattaagggcccggccgtgacggccacgagcgaactcctgcaggtgtttaaactagggataacagggtaataggtctcacgcggcaaatcctaccacctcatttaaatagagtgaggttgatttgcggccgcacaacaaacgccggcgctctcctctcttaaggtagccggtgacacccttgggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaa

SEQ ID NO: 10 - DNA, 14240 bp, synthetic construct
  source 1..14240: mol_type = other DNA; note = A 14,240-nucleotide sequence corresponding to the contig nucleotide sequence of the 5' genomic flanking DNA nucleotide sequence, the inserted T-DNA nucleotide sequence in event GM_CSM63770, and the 3' genomic flanking DNA nucleotide sequence.
ataataacacaaaagagaatcaaaatgtttattggaaccagctctattcttagtttatttttattttggtgatatatttttaatttgttagaaataaatggtgtaacagttttaattagttttaatggtttgaacagttaattataatgaatgtatgagatgtgtgtactttattctccaaactctaaagtactgactttaaataaaaaatagttatcattgaaaaggtgttcatttttataactgttaacctataacttcttctatagttttgggtacaaaaactgttcacgtacgtacgtattcttataactaactgttaacctataacttgtttttctctcttcatcatattttaatttccgttttttcccctcatttctgtttctttgtatcgtagaaacatagactccgaccaatgccaaaatctttgatcttcattgttcatattctccacatctcaatcgattgttgcagaatcacagaaggagaaagaacataaagaaaagcttgcatttttgcctcaatatcatgttatttttgtttttggaggggagagataactacactaatctcttacgagatcagtgttaatattactacaaaaaagtgataatgatatttacatttttttaagtaaaacatatacatcttctaaaaaaaaagtaaaatatatacatatctctaatttatatttacatacgcatgcaatgttaggtatattcagcaatgcaacactattttcttttttctctataccgttttttctttattattgcatggatagtaggaaaaaataaataattaaaccaaaattaatatataagtcaatatattaaagataaattgttactagcaactttagcataataaaaatgggatatataattttagtgcagtcaaaattgtgataatatattaaatttagcataaaaagaaatgcaaataccccgttgtagtgtctgtttctcttagtacatatataataaaaccttgaattagtcagcatcatcacaccaaaagttaggcccgaatagtttgaaattagaaagctcgcaattgaggtctgtcgaccctgcactaactataacggtcctaaggtagcgacttaggctgagcccgggcaggcctacccataatacccataatagctgtttgccaatcgttcttcttggcgcgccgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtggtaccaccatggagattgtgaacaaccagaaccagtgcgttccttacaactgcttgaacaaccctgagattgagattcttgagggtggtagaatttctgttggcaacactcctattgacatctctttgagtttgactcaattcttgttgagtgagttcgttcctggtgctggtttcgtcttgggtttgattgatttgatttggggtttcgttggtcctagtcaatgggatgctttcttggctcaagttgagcaattgattaaccagaggatcgctgaggctgtgaggaacactgctattcaagagttggagggtatggctagagtttacagaacttacgctactgctttcgctgagtgggagaaggctcctgatgaccctgagttgagggaggctttgagaactcaattcactgctactgagacttacatcagtggtagaatcagtgtcttgaagattcaaactttcgaggttcaattgctttctgtgttcgctcaagctgcaaacttgcacttgtctttgcttagagatgttgtgttctttggtcaaagatggggtttctccactactaccgtgaacaattactacaacgatttgactgagggtatttctacttacactgattacgctgttagatggtacaacactggtttggagagagtttggggtccagattccagagattgggtcagatacaaccagttcagaagggagttgactttgactgtcttggacattgttgctctcttccctaactacgatagtcgtcgttaccctattagaactgtttctcaacttactagggaaatctacactaaccctgttcttgagaacttcgatggtagtttccgtggtagtgctcaagggattgagcgttctattcgttctcctcatcttatggacattcttaactctattactatctacactgatgctcatcgtggttactattactggtctggtcatcaaattatggctagtcctgttggtttcagtggtcctgagttcactttccctctttacggtactatgggcaacgctgcacctcaacagaggatcgttgctcaacttggtcaaggtgtttacaggactctttcttcaaccctttacaggcgtcctttcaacattgggatcaacaaccagcagctttctgttcttgatggaaccgagttcgcttacggaacctcttcaaaccttcctagtgctgtttacaggaagtctggaaccgttgacagtcttgatgagattccaccgcagaacaataacgttccacccaggcaaggcttcagtcataggctttctcatgtttctatgttccgctctggattcagcaactcttcagtttctattatcagggctccaatgttctcgtggattcataggtctgccgagttcaacaacattatcgcttccgatagcattaaccagattccacttgttaagggattccgtgtttggggaggcacctctgttattaccggaccaggcttcaccggaggcgacattcttcgtcgtaacaccttcggagatttcgtttcacttcaagtgaacattaactcaccaatcacccagcgctacaggcttcgcttccgctacgcatcatccagggatgcaagggtgatcgtgcttaccggagcagcctcaaccggagtgggaggccaagtgagcgtgaacatgccacttcagaagacgatggagatcggcgagaaccttacctcaagaacctttcgttacaccgatttcagcaacccattcagctttcgtgcaaacccagacatcatagggatctcagagcagccactgtttggagctggatcaatctcatccggagagctttacatcgacaagatcgagatcatactcgcagatgcaaccttcgaggctgagagcgatctggagcgtgcacagaaggcagtgaacgcactctttacctctaccaaccagctcggactcaagaccaacgtgaccgattaccacatcgaccaagtgagcaacctcgtgacctacctctcagatgagttctgcttggatgagaaacgcgaactcagcgagaaggtgaagcacgcaaagcgtctctcagatgagcgtaacctcctccaggatagcaatttcaaggacatcaatcgtcagccagagcgtggatggggaggctcaaccggaatcaccatccagggaggcgatgatgtgtttaaggagaattacgtgacactctccggaacattcgatgagtgctacccaacatacctctatcagaagatcgacgagtccaagctcaaggcgttcacccgttatcagctccgtggctacatcgaggatagtcaagacctggaaatctacctcatccgctacaatgcaaagcacgagacagtgaatgtaccaggaacaggctccctctggccactctccgcacagtctccaatcggcaagtgcggcgagccaaatcgctgcgcgccacacctggagtggaatcccgacctggactgctcctgccgcgacggcgagaagtgcgcccaccactcccaccactttagcctggacatcgacgtgggctgtacagacctgaatgaggatctgggcgtgtgggtgatctttaagatcaagacacaggacggccacgcccgcctgggcaatctggagtttctggaggagaagcctctggtgggcgaagccctggcccgcgtgaagcgcgccgagaagaaatggcgcgacaaacgcgagaaactggaatgggaaacaaacatcgtgtacaaagaagccaaagaatccgtggacgccctatttgtgaactcccagtatgaccagctacaggccgacacaaacatcgcgatgatccacgctgcggacaagcgcgtgcactccatacgcgaagcctatctacccgaactatccgtgatacccggcgtcaatgccgcgatctttgaagaattggaaggccgcatcttcacagcctttagcctctatgacgcccgaaatgtcatcaagaatggcgactttaacaatgggctatcctgttggaatgtcaaagggcacgtggacgtcgaagagcagaacaatcagcgatccgtcttagtcgtacccgaatgggaagccgaagtctcccaggaagtccgagtctgtcctggtagaggttacatcttgagagtgactgcttacaaggagggttacggtgagggatgcgtgactattcacgagattgagaacaacactgatgagttgaagttcagtaactgcgtggaggaggaaatctaccccaacaacactgtgacttgtaacgattacaccgtgaaccaggaggaatacggaggcgcttacacctccagaaaccgtggatacaatgaggctccctcggtccccgctgattatgcctccgtctatgaggagaagtcctacaccgatggaaggcgcgagaatccctgcgagttcaatcgcggctatcgagactacactccgctacccgttggctatgtcacaaaggaactggaatacttcccggaaacagacaaagtctggatcgaaatcggcgaaacagaagggacgttcatagtcgatagcgtagaacttctccttatggaagaatgacctgcaggtcattcaatttgacccttttgctacattcttgctgtcatagttatttaatttaatttcttaaggtgcatattcttttggtgaatttaagcccccccgaaacatggaaggaggttggctgcaaagttcctgaaggataatcgtttttttcattccctttaatttttgttattcattatttcacattttacctaaaacttcagggagtgagataaagaactctttctctctgaactgtttatgttgtctactagaaaaaaaaatcctgtaagaggtaaggtattcttccaaaacaggattctttttcttatcattttacttcattagtttctaagttccttttcctacattgtataagattctgtcagtacatttatgttgcttatgtaggaagtgggactgagtttcttatgaaggaatttatgagttattgaatatttattgcaagctatattttagaatacagtatactttcttcttatgttattatttttttaagtgtattgtgttgtgttattattataagctttgttaaactctgatcattttcaaattcaaaacaatgtttagttcaaaggcaaagctgagttgtgctgattatttagggataatagctgtttgccaatcgttcttcttggcgcgccacgtcctgcttggcctactaggcccggaccgacggtacctcagcgctgtgcctgttgcgaaaaacccaaataaatacaaagttaatagattttcaacaatgcggggatagctcagttgggagagcgtcagactgaagatctgaaggtcgcgtgttcgatccacgctcaccgcatcttttttttgttctgtttttcccagcgaatgaactatccatacgtcgtcgtttgtatagccatgatccatgaagcgctctaattaccaatcattttttctttcttggaattgatcaagttacaaatataaaattattttacactaaaaactctcttttaaaaatttagatccaattttaattttgttttctaaaagattgtatccaagaagtaaaatttacatggaaaataaaatgcctataaattaaaaaatgttatattttcgtgacctttaaccttaggaacatttgataactattactttgagattgcagtcataggcaacacaaggtcttggacaaacatgttcgtgacaccaaacacccccctcaaaattaagtatattataccaactcatttcatttgcttgttacggttgtgttttatcaatccttcctcgtcttgttttttcctccaaatttgtaatcttaaaattggtgaaacatcacatatttgattctcaatcttagattttctaccttttaggtaatctatattttgaaattgaaattgaaattgaattccgtgtaaaatctataatatgggaacccaattcgaatttaatgttttatgtaaaggccaataatgttccaaatttatttccttattattattgtatattttgtccccaagtacatcatagattggctttcaattccaaatccatcatggtatatttttaggtgcaaactattgtcgtagatttaatcccctagttttgttgattttttaaaatagtactttcaacaattttaaattttatttgattggttataaacttcgggtgtgatttagttgtccaatttggattatattgtcattttcatacataaatttatttatttttgaaaaagttatcttttagtctttgaaattgaaatttttatgaatatgtggatttagtccctcaattctcaaattaaccatcttcacaagagtgagaaacagagatgggagggagaaaaatggaaagataaaaaaagtatttttcttcctccatgttattaggtcacatcaatatttataatattttatattaaatttaatgatttcttttgggagaggtactcttagacctatttttttaacaaaaattattggactcataattttttaattgtcctttaatttcttttatttgtatctagactttataaagtttgagagttttatattttaaggagaaatatattaaaatataaggttagttttcatacactagatagacttatgatcttatacgagtgaatgagttaaactctcaattaaaattatttgaattttttcaaaaattaaaatcatccattttgttaaattaaaatttgaagaaatttataggatgcgtaagaattgatgccttgttaattttcaaatataattatagtaaaaattcttaattagtttactaacaacgatttaaaagtttactttgatgtgttgaacaatatttatcaaaccaaatttgatcaagtggatatactaacaaaagttcaagatcttaaactttaagtgaattaattttgacatatatatatatataaaaaaaaaaagttaggtgttaaaagaaaactgaaaattgttaaaagaaaagaaaaggagggtatagataaaatgtggatagaaagagatgagttgtagatgcaaaaaaatctttagaaacaatatgattggctaactaagccctctcattaccccacaaaatttctcatatctttcactcttcaccactcatctcctcgtttcctcataatattcccccacttccactctctctcacacaatctccaacactactgcccattcctctgttctttctctctactctcaaccacagagcttcaagtacccaccatgaccagcaaccgaaagaacgagaacgagatcatcaacgccctgtccataccggccgtgtcaaaccactccgcccagatgaacctctccaccgacgcgaggatcgaggactccctctgcatcgccgagggcaacaacatcgacccgttcgtgtctgcaagcacggtccagaccggcatcaacatcgcgggccgcatcctgggcgtgctcggcgtgcccttcgcgggtcaaatcgcctctttctactcattcctcgtgggcgagctgtggccgcgcggacgtgacccgtgggaaatcttcctggagcacgttgagcagctcatccggcagcaagtgaccgagaacaccagggacaccgcactggcacggctccagggccttggcaacagcttccgcgcctaccagcagtcgctggaggactggctggagaaccgagacgacgccagaacccgctcagttctgtacacacagtacatcgccctagagctggacttcctcaacgctatgccgctcttcgccatccgtaaccaggaagtaccgcttctgatggtgtacgcacaagcagcgaacctccatctgctcctgctgcgagacgcatctctgttcggcagtgagttcgggctgacgagccaggagatccagcgctactacgagcgccaagtggagaagactcgtgagtacagcgactactgcgcgcgctggtacaacacgggcttgaacaaccttcgcgggacaaacgccgaatcctggcttcgctacaaccagttccgccgcgacctcacgctgggtgtgctggacctggtcgcgctcttcccgtcctacgacacacgggtgtacccaatgaacacgagcgcacagctcacccgtgagatctacacagatcccatcggccgcaccaacgctcccagtggcttcgcaagcacgaattggttcaacaataacgctccttctttctctgccatcgaggccgctgtcatcagaccgccgcacttactcgatttcccggagcagctcactatcttctctgtgttgtcccggtggtcgaacacgcagtacatgaactactgggtgggccacaggctagagagccggaccatccgtggcagtctctcaacctcgacccacggcaacacgaacacgagcatcaaccctgtcactctccagtttacatctagggacgtttacaggacagagtcgttcgctggcattaacattctgttgaccactccggtgaacggcgtcccttgggcccgcttcaactggaggaatcctctgaactcactgcgcggcagccttctctacactatcggctacaccggcgttgggacgcaactcttcgactcggagaccgagctgccgcccgagaccaccgagcggcctaactacgagagttattcacacaggctctccaacatccgcttgatttctgggaacaccttgcgggctccggtgtactcctggacgcaccgcagcgccgacagaactaataccatcagctccgactcgatcacccagatcccgctggtgaaggctcacacgcttcagtcgggcaccacagtcgtcaagggccctggcttcaccggcggcgacatcctgcgtcgcacatctggcggacccttcgccttcagcaacgtgaacttggacttcaatttgtcacagcggtatcgtgccagaatccggtacgccagcactacgaacctgcgaatctatgttactgtggcgggcgagcggatcttcgccgggcaattcgacaagacgatggacgcgggagcacctctgacattccagtcattctcttacgccacgatcaacacggcattcacgtttccggagcgttccagtagcctgaccgtgggcgctgataccttcagtagcgggaacgaggtgtacgttgaccgtttcgagctgatcccggtcaccgccaccttcgaagctgagtcggacctggagcgtgcacagaaggcagtcaacgagctgttcacctctagcaaccagatcggcctcaagaccgacgtcacagactaccacatcgaccaagtgtccaacctggtcgagtgccttagcgacgagttctgcctagacgagaagaaggagctgtcggagaaggtcaaacacgccaagcgtctgagcgatgagcgcaacctgctccaagaccctaacttccgtggcatcaacaggcagcttgaccgtggctggcgcggctcgacggacatcacgatccagggtggcgacgacgtattcaaggagaattacgtgaccttgcttgggacgtttgacgagtgctatcccacctacctctaccagaagattgatgaatcgaaattgaaggcgtacacgagataccagctccgtggctacatcgaggacagccaggacttggagatctacctcatacgctacaacgctaaacatgagaccgtgaacgtccctgggacgggcagtctgtggccactctctgctcctagccctatcggcaagtgcgctcaccactcgcaccacttcagccttgacatcgacgtgggatgtactgacctcaacgaagacctgggcgtctgggttatcttcaagatcaagacccaggacggccacgcccgactcggcaacctggagttcctggaggagaaaccactggtgggcgaggcgctcgcccgcgtgaagcgtgccgagaagaagtggcgggacaagagggagaagctagaatgggagacgaacatcgtgtacaaggaggccaaggaaagcgtcgatgccctgttcgtgaactcacagtacgaccgtctccaggcggacacgaacatcgccatgatccacgcggctgacaagcgcgtccactccatccgcgaggcgtacttaccggagctgtcggtgatcccaggcgtaaacgcggcgatcttcgaggagctagagggacgcatcttcacagcgttcagcctgtacgacgcacgcaacgtcatcaagaacggcgatttcaacaacggactgtcctgctggaacgtgaagggccacgtcgatgtcgaggaacagaacaaccaccgctctgtcctggtggtcccagagtgggaggccgaggtctcccaggaggtccgcgtgtgccctgggcgtggctacatcctccgtgtgacagcctacaaggagggctacggtgagggctgcgtcaccattcacgagatcgagaacaacactgacgaactcaagttctcgaattgcgtggaggaggaggtgtacccgaacaatacggtgacgtgcaacgactacacggcaacccaagaggagtacgagggcacctacaccagtaggaaccgtggctacgacggtgcctacgagtcgaactccagcgtccctgcggactacgccagcgcgtacgaggagaaggcttacaccgacggacgccgggacaacccatgcgagagcaaccgtggctacggcgactacactcctctcccggccggatacgtcacaaaggagctggagtatttcccagagacggacaaggtgtggatcgaaatcggagagacagagggaaccttcatcgtggacagcgtggagctgctcctcatggaggagtgagaaaactagtggtttatttactatatgttattgtatctttcaataaagagatgctccatttgaccaaaaataaaaagagatgctccagatagtgcatcttatctatgttataataatcaagtgtatgcatttccttcccctttatttggagcatgtgtatgtttaaatatttgtaatttgaaccttcaaacttgttctagttaccaaataataagatcattattagtttgtgtgtttatctctattgtctactaattttattttttttttacttttctattctttatttgtattaaatcataagttctaattatttaatcaaaacttcaaatggtggcggttatacgctattccgaatcgtcttttgttgtccgagtctataggctacactcatatcctggttgaaacagactctatagctgtcctgatcaggtcaatcagattagaagattcttttctcaaaattggagtcttttctttcaacgctgtgcagattgacttcgccagcagaacacgcgctgaggttaattaagggcccggccgtgacggccacgagcgaactcctgcaggtgtttaaactagggataacagggtaataggtctcacgcggcaaatcctaccacctcatttaaatagagtgaggttgatttgcggccgcacaacaaacgccggcgctctcctctcttaaggtagccggtgacacccttgggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaaccatagatgtgaggaagaatgtcaaaaatggaataaattttataaccactagcagaaatagaagcactacaatgtataactgcctttcccatgttttatttgtttcgtatgatctaagatgttaagttgacatgtttattaatcttgaagcactattaatttatttctacttatctgtacttttttttattcttaatttaataattttttcctcaatatattttaaaaatatatattaaactaaaattaatataaatgttaaagcatgataaaataaaatgttttgatatatcaaatttaacaaataaaaatacggacacacaaataagataacacatgtgtttttcattttgctaagtatatatattattgatcatatatcttatgattcatgatttgattcacgattccatacataggctttccgattcaactaccttgctggtaattatattgtctcacaccaagcatcaaaattatttgcaattacaaattaattgtttgctagttgttactgatgtcaatggtaaggaggcgttccgaggatctccttcgtttacttttgggctccatatcttccaaatgatgagtgatattgtcactattgatcaattttcactttaaaactgaatcattctgattaaaaaattaattcaaattaagtaactgattgaatttaaaattatatggaaaacaaagatatagattatgaatgagttgtcgctttgcttgttgttattgttaagtgaatttgggttcattgatttcaatgccgctggaatcaaatttgtaagtgaatttggagagattcttggataaatgtatcatatatttcatagtagcatgcatctcattctttgatttgggataaattagcaatttttgctattttacaggtccaatatgttcctgaacaattagaatcactaagggtacgtagtcctttgataactgccacatatatggagctcgaagatggactgagtagtagggaatggag

SEQ ID NO: 11 - DNA, 1000 bp, Glycine max
  source 1..1000: mol_type = genomic DNA; note = A 1,000-nucleotide sequence representing the 5' flanking soybean genomic DNA up to the inserted T-DNA.
ataataacacaaaagagaatcaaaatgtttattggaaccagctctattcttagtttatttttattttggtgatatatttttaatttgttagaaataaatggtgtaacagttttaattagttttaatggtttgaacagttaattataatgaatgtatgagatgtgtgtactttattctccaaactctaaagtactgactttaaataaaaaatagttatcattgaaaaggtgttcatttttataactgttaacctataacttcttctatagttttgggtacaaaaactgttcacgtacgtacgtattcttataactaactgttaacctataacttgtttttctctcttcatcatattttaatttccgttttttcccctcatttctgtttctttgtatcgtagaaacatagactccgaccaatgccaaaatctttgatcttcattgttcatattctccacatctcaatcgattgttgcagaatcacagaaggagaaagaacataaagaaaagcttgcatttttgcctcaatatcatgttatttttgtttttggaggggagagataactacactaatctcttacgagatcagtgttaatattactacaaaaaagtgataatgatatttacatttttttaagtaaaacatatacatcttctaaaaaaaaagtaaaatatatacatatctctaatttatatttacatacgcatgcaatgttaggtatattcagcaatgcaacactattttcttttttctctataccgttttttctttattattgcatggatagtaggaaaaaataaataattaaaccaaaattaatatataagtcaatatattaaagataaattgttactagcaactttagcataataaaaatgggatatataattttagtgcagtcaaaattgtgataatatattaaatttagcataaaaagaaatgcaaataccccgttgtagtgtctgtttctcttagtacatatataataaaaccttgaatt

SEQ ID NO: 12 - DNA, 1000 bp, Glycine max
  source 1..1000: mol_type = genomic DNA; note = A 1,000-nucleotide sequence representing the 3' flanking soybean genomic DNA after the inserted T-DNA.
ccatagatgtgaggaagaatgtcaaaaatggaataaattttataaccactagcagaaatagaagcactacaatgtataactgcctttcccatgttttatttgtttcgtatgatctaagatgttaagttgacatgtttattaatcttgaagcactattaatttatttctacttatctgtacttttttttattcttaatttaataattttttcctcaatatattttaaaaatatatattaaactaaaattaatataaatgttaaagcatgataaaataaaatgttttgatatatcaaatttaacaaataaaaatacggacacacaaataagataacacatgtgtttttcattttgctaagtatatatattattgatcatatatcttatgattcatgatttgattcacgattccatacataggctttccgattcaactaccttgctggtaattatattgtctcacaccaagcatcaaaattatttgcaattacaaattaattgtttgctagttgttactgatgtcaatggtaaggaggcgttccgaggatctccttcgtttacttttgggctccatatcttccaaatgatgagtgatattgtcactattgatcaattttcactttaaaactgaatcattctgattaaaaaattaattcaaattaagtaactgattgaatttaaaattatatggaaaacaaagatatagattatgaatgagttgtcgctttgcttgttgttattgttaagtgaatttgggttcattgatttcaatgccgctggaatcaaatttgtaagtgaatttggagagattcttggataaatgtatcatatatttcatagtagcatgcatctcattctttgatttgggataaattagcaatttttgctattttacaggtccaatatgttcctgaacaattagaatcactaagggtacgtagtcctttgataactgccacatatatggagctcgaagatggactgagtagtagggaatggag

SEQ ID NO: 13 - DNA, 12629 bp, synthetic construct
  source 1..12629: mol_type = other DNA; note = A 12,629-nucleotide sequence representing the transgene cassette comprised within the binary plasmid transformation vector used to transform soybean to produce soybean event GM_CSM63770.
cgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctgtcgaccctgcactaactataacggtcctaaggtagcgacttaggctgagcccgggcaggcctacccataatacccataatagctgtttgccaatcgttcttcttggcgcgccgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtggtaccaccatggagattgtgaacaaccagaaccagtgcgttccttacaactgcttgaacaaccctgagattgagattcttgagggtggtagaatttctgttggcaacactcctattgacatctctttgagtttgactcaattcttgttgagtgagttcgttcctggtgctggtttcgtcttgggtttgattgatttgatttggggtttcgttggtcctagtcaatgggatgctttcttggctcaagttgagcaattgattaaccagaggatcgctgaggctgtgaggaacactgctattcaagagttggagggtatggctagagtttacagaacttacgctactgctttcgctgagtgggagaaggctcctgatgaccctgagttgagggaggctttgagaactcaattcactgctactgagacttacatcagtggtagaatcagtgtcttgaagattcaaactttcgaggttcaattgctttctgtgttcgctcaagctgcaaacttgcacttgtctttgcttagagatgttgtgttctttggtcaaagatggggtttctccactactaccgtgaacaattactacaacgatttgactgagggtatttctacttacactgattacgctgttagatggtacaacactggtttggagagagtttggggtccagattccagagattgggtcagatacaaccagttcagaagggagttgactttgactgtcttggacattgttgctctcttccctaactacgatagtcgtcgttaccctattagaactgtttctcaacttactagggaaatctacactaaccctgttcttgagaacttcgatggtagtttccgtggtagtgctcaagggattgagcgttctattcgttctcctcatcttatggacattcttaactctattactatctacactgatgctcatcgtggttactattactggtctggtcatcaaattatggctagtcctgttggtttcagtggtcctgagttcactttccctctttacggtactatgggcaacgctgcacctcaacagaggatcgttgctcaacttggtcaaggtgtttacaggactctttcttcaaccctttacaggcgtcctttcaacattgggatcaacaaccagcagctttctgttcttgatggaaccgagttcgcttacggaacctcttcaaaccttcctagtgctgtttacaggaagtctggaaccgttgacagtcttgatgagattccaccgcagaacaataacgttccacccaggcaaggcttcagtcataggctttctcatgtttctatgttccgctctggattcagcaactcttcagtttctattatcagggctccaatgttctcgtggattcataggtctgccgagttcaacaacattatcgcttccgatagcattaaccagattccacttgttaagggattccgtgtttggggaggcacctctgttattaccggaccaggcttcaccggaggcgacattcttcgtcgtaacaccttcggagatttcgtttcacttcaagtgaacattaactcaccaatcacccagcgctacaggcttcgcttccgctacgcatcatccagggatgcaagggtgatcgtgcttaccggagcagcctcaaccggagtgggaggccaagtgagcgtgaacatgccacttcagaagacgatggagatcggcgagaaccttacctcaagaacctttcgttacaccgatttcagcaacccattcagctttcgtgcaaacccagacatcatagggatctcagagcagccactgtttggagctggatcaatctcatccggagagctttacatcgacaagatcgagatcatactcgcagatgcaaccttcgaggctgagagcgatctggagcgtgcacagaaggcagtgaacgcactctttacctctaccaaccagctcggactcaagaccaacgtgaccgattaccacatcgaccaagtgagcaacctcgtgacctacctctcagatgagttctgcttggatgagaaacgcgaactcagcgagaaggtgaagcacgcaaagcgtctctcagatgagcgtaacctcctccaggatagcaatttcaaggacatcaatcgtcagccagagcgtggatggggaggctcaaccggaatcaccatccagggaggcgatgatgtgtttaaggagaattacgtgacactctccggaacattcgatgagtgctacccaacatacctctatcagaagatcgacgagtccaagctcaaggcgttcacccgttatcagctccgtggctacatcgaggatagtcaagacctggaaatctacctcatccgctacaatgcaaagcacgagacagtgaatgtaccaggaacaggctccctctggccactctccgcacagtctccaatcggcaagtgcggcgagccaaatcgctgcgcgccacacctggagtggaatcccgacctggactgctcctgccgcgacggcgagaagtgcgcccaccactcccaccactttagcctggacatcgacgtgggctgtacagacctgaatgaggatctgggcgtgtgggtgatctttaagatcaagacacaggacggccacgcccgcctgggcaatctggagtttctggaggagaagcctctggtgggcgaagccctggcccgcgtgaagcgcgccgagaagaaatggcgcgacaaacgcgagaaactggaatgggaaacaaacatcgtgtacaaagaagccaaagaatccgtggacgccctatttgtgaactcccagtatgaccagctacaggccgacacaaacatcgcgatgatccacgctgcggacaagcgcgtgcactccatacgcgaagcctatctacccgaactatccgtgatacccggcgtcaatgccgcgatctttgaagaattggaaggccgcatcttcacagcctttagcctctatgacgcccgaaatgtcatcaagaatggcgactttaacaatgggctatcctgttggaatgtcaaagggcacgtggacgtcgaagagcagaacaatcagcgatccgtcttagtcgtacccgaatgggaagccgaagtctcccaggaagtccgagtctgtcctggtagaggttacatcttgagagtgactgcttacaaggagggttacggtgagggatgcgtgactattcacgagattgagaacaacactgatgagttgaagttcagtaactgcgtggaggaggaaatctaccccaacaacactgtgacttgtaacgattacaccgtgaaccaggaggaatacggaggcgcttacacctccagaaaccgtggatacaatgaggctccctcggtccccgctgattatgcctccgtctatgaggagaagtcctacaccgatggaaggcgcgagaatccctgcgagttcaatcgcggctatcgagactacactccgctacccgttggctatgtcacaaaggaactggaatacttcccggaaacagacaaagtctggatcgaaatcggcgaaacagaagggacgttcatagtcgatagcgtagaacttctccttatggaagaatgacctgcaggtcattcaatttgacccttttgctacattcttgctgtcatagttatttaatttaatttcttaaggtgcatattcttttggtgaatttaagcccccccgaaacatggaaggaggttggctgcaaagttcctgaaggataatcgtttttttcattccctttaatttttgttattcattatttcacattttacctaaaacttcagggagtgagataaagaactctttctctctgaactgtttatgttgtctactagaaaaaaaaatcctgtaagaggtaaggtattcttccaaaacaggattctttttcttatcattttacttcattagtttctaagttccttttcctacattgtataagattctgtcagtacatttatgttgcttatgtaggaagtgggactgagtttcttatgaaggaatttatgagttattgaatatttattgcaagctatattttagaatacagtatactttcttcttatgttattatttttttaagtgtattgtgttgtgttattattataagctttgttaaactctgatcattttcaaattcaaaacaatgtttagttcaaaggcaaagctgagttgtgctgattatttagggataatagctgtttgccaatcgttcttcttggcgcgccacgtcctgcttggcctactaggcccggaccgacggtacctcagcgctgtgcctgttgcgaaaaacccaaataaatacaaagttaatagattttcaacaatgcggggatagctcagttgggagagcgtcagactgaagatctgaaggtcgcgtgttcgatccacgctcaccgcatcttttttttgttctgtttttcccagcgaatgaactatccatacgtcgtcgtttgtatagccatgatccatgaagcgctctaattaccaatcattttttctttcttggaattgatcaagttacaaatataaaattattttacactaaaaactctcttttaaaaatttagatccaattttaattttgttttctaaaagattgtatccaagaagtaaaatttacatggaaaataaaatgcctataaattaaaaaatgttatattttcgtgacctttaaccttaggaacatttgataactattactttgagattgcagtcataggcaacacaaggtcttggacaaacatgttcgtgacaccaaacacccccctcaaaattaagtatattataccaactcatttcatttgcttgttacggttgtgttttatcaatccttcctcgtcttgttttttcctccaaatttgtaatcttaaaattggtgaaacatcacatatttgattctcaatcttagattttctaccttttaggtaatctatattttgaaattgaaattgaaattgaattccgtgtaaaatctataatatgggaacccaattcgaatttaatgttttatgtaaaggccaataatgttccaaatttatttccttattattattgtatattttgtccccaagtacatcatagattggctttcaattccaaatccatcatggtatatttttaggtgcaaactattgtcgtagatttaatcccctagttttgttgattttttaaaatagtactttcaacaattttaaattttatttgattggttataaacttcgggtgtgatttagttgtccaatttggattatattgtcattttcatacataaatttatttatttttgaaaaagttatcttttagtctttgaaattgaaatttttatgaatatgtggatttagtccctcaattctcaaattaaccatcttcacaagagtgagaaacagagatgggagggagaaaaatggaaagataaaaaaagtatttttcttcctccatgttattaggtcacatcaatatttataatattttatattaaatttaatgatttcttttgggagaggtactcttagacctatttttttaacaaaaattattggactcataattttttaattgtcctttaatttcttttatttgtatctagactttataaagtttgagagttttatattttaaggagaaatatattaaaatataaggttagttttcatacactagatagacttatgatcttatacgagtgaatgagttaaactctcaattaaaattatttgaattttttcaaaaattaaaatcatccattttgttaaattaaaatttgaagaaatttataggatgcgtaagaattgatgccttgttaattttcaaatataattatagtaaaaattcttaattagtttactaacaacgatttaaaagtttactttgatgtgttgaacaatatttatcaaaccaaatttgatcaagtggatatactaacaaaagttcaagatcttaaactttaagtgaattaattttgacatatatatatatataaaaaaaaaaagttaggtgttaaaagaaaactgaaaattgttaaaagaaaagaaaaggagggtatagataaaatgtggatagaaagagatgagttgtagatgcaaaaaaatctttagaaacaatatgattggctaactaagccctctcattaccccacaaaatttctcatatctttcactcttcaccactcatctcctcgtttcctcataatattcccccacttccactctctctcacacaatctccaacactactgcccattcctctgttctttctctctactctcaaccacagagcttcaagtacccaccatgaccagcaaccgaaagaacgagaacgagatcatcaacgccctgtccataccggccgtgtcaaaccactccgcccagatgaacctctccaccgacgcgaggatcgaggactccctctgcatcgccgagggcaacaacatcgacccgttcgtgtctgcaagcacggtccagaccggcatcaacatcgcgggccgcatcctgggcgtgctcggcgtgcccttcgcgggtcaaatcgcctctttctactcattcctcgtgggcgagctgtggccgcgcggacgtgacccgtgggaaatcttcctggagcacgttgagcagctcatccggcagcaagtgaccgagaacaccagggacaccgcactggcacggctccagggccttggcaacagcttccgcgcctaccagcagtcgctggaggactggctggagaaccgagacgacgccagaacccgctcagttctgtacacacagtacatcgccctagagctggacttcctcaacgctatgccgctcttcgccatccgtaaccaggaagtaccgcttctgatggtgtacgcacaagcagcgaacctccatctgctcctgctgcgagacgcatctctgttcggcagtgagttcgggctgacgagccaggagatccagcgctactacgagcgccaagtggagaagactcgtgagtacagcgactactgcgcgcgctggtacaacacgggcttgaacaaccttcgcgggacaaacgccgaatcctggcttcgctacaaccagttccgccgcgacctcacgctgggtgtgctggacctggtcgcgctcttcccgtcctacgacacacgggtgtacccaatgaacacgagcgcacagctcacccgtgagatctacacagatcccatcggccgcaccaacgctcccagtggcttcgcaagcacgaattggttcaacaataacgctccttctttctctgccatcgaggccgctgtcatcagaccgccgcacttactcgatttcccggagcagctcactatcttctctgtgttgtcccggtggtcgaacacgcagtacatgaactactgggtgggccacaggctagagagccggaccatccgtggcagtctctcaacctcgacccacggcaacacgaacacgagcatcaaccctgtcactctccagtttacatctagggacgtttacaggacagagtcgttcgctggcattaacattctgttgaccactccggtgaacggcgtcccttgggcccgcttcaactggaggaatcctctgaactcactgcgcggcagccttctctacactatcggctacaccggcgttgggacgcaactcttcgactcggagaccgagctgccgcccgagaccaccgagcggcctaactacgagagttattcacacaggctctccaacatccgcttgatttctgggaacaccttgcgggctccggtgtactcctggacgcaccgcagcgccgacagaactaataccatcagctccgactcgatcacccagatcccgctggtgaaggctcacacgcttcagtcgggcaccacagtcgtcaagggccctggcttcaccggcggcgacatcctgcgtcgcacatctggcggacccttcgccttcagcaacgtgaacttggacttcaatttgtcacagcggtatcgtgccagaatccggtacgccagcactacgaacctgcgaatctatgttactgtggcgggcgagcggatcttcgccgggcaattcgacaagacgatggacgcgggagcacctctgacattccagtcattctcttacgccacgatcaacacggcattcacgtttccggagcgttccagtagcctgaccgtgggcgctgataccttcagtagcgggaacgaggtgtacgttgaccgtttcgagctgatcccggtcaccgccaccttcgaagctgagtcggacctggagcgtgcacagaaggcagtcaacgagctgttcacctctagcaaccagatcggcctcaagaccgacgtcacagactaccacatcgaccaagtgtccaacctggtcgagtgccttagcgacgagttctgcctagacgagaagaaggagctgtcggagaaggtcaaacacgccaagcgtctgagcgatgagcgcaacctgctccaagaccctaacttccgtggcatcaacaggcagcttgaccgtggctggcgcggctcgacggacatcacgatccagggtggcgacgacgtattcaaggagaattacgtgaccttgcttgggacgtttgacgagtgctatcccacctacctctaccagaagattgatgaatcgaaattgaaggcgtacacgagataccagctccgtggctacatcgaggacagccaggacttggagatctacctcatacgctacaacgctaaacatgagaccgtgaacgtccctgggacgggcagtctgtggccactctctgctcctagccctatcggcaagtgcgctcaccactcgcaccacttcagccttgacatcgacgtgggatgtactgacctcaacgaagacctgggcgtctgggttatcttcaagatcaagacccaggacggccacgcccgactcggcaacctggagttcctggaggagaaaccactggtgggcgaggcgctcgcccgcgtgaagcgtgccgagaagaagtggcgggacaagagggagaagctagaatgggagacgaacatcgtgtacaaggaggccaaggaaagcgtcgatgccctgttcgtgaactcacagtacgaccgtctccaggcggacacgaacatcgccatgatccacgcggctgacaagcgcgtccactccatccgcgaggcgtacttaccggagctgtcggtgatcccaggcgtaaacgcggcgatcttcgaggagctagagggacgcatcttcacagcgttcagcctgtacgacgcacgcaacgtcatcaagaacggcgatttcaacaacggactgtcctgctggaacgtgaagggccacgtcgatgtcgaggaacagaacaaccaccgctctgtcctggtggtcccagagtgggaggccgaggtctcccaggaggtccgcgtgtgccctgggcgtggctacatcctccgtgtgacagcctacaaggagggctacggtgagggctgcgtcaccattcacgagatcgagaacaacactgacgaactcaagttctcgaattgcgtggaggaggaggtgtacccgaacaatacggtgacgtgcaacgactacacggcaacccaagaggagtacgagggcacctacaccagtaggaaccgtggctacgacggtgcctacgagtcgaactccagcgtccctgcggactacgccagcgcgtacgaggagaaggcttacaccgacggacgccgggacaacccatgcgagagcaaccgtggctacggcgactacactcctctcccggccggatacgtcacaaaggagctggagtatttcccagagacggacaaggtgtggatcgaaatcggagagacagagggaaccttcatcgtggacagcgtggagctgctcctcatggaggagtgagaaaactagtggtttatttactatatgttattgtatctttcaataaagagatgctccatttgaccaaaaataaaaagagatgctccagatagtgcatcttatctatgttataataatcaagtgtatgcatttccttcccctttatttggagcatgtgtatgtttaaatatttgtaatttgaaccttcaaacttgttctagttaccaaataataagatcattattagtttgtgtgtttatctctattgtctactaattttattttttttttacttttctattctttatttgtattaaatcataagttctaattatttaatcaaaacttcaaatggtggcggttatacgctattccgaatcgtcttttgttgtccgagtctataggctacactcatatcctggttgaaacagactctatagctgtcctgatcaggtcaatcagattagaagattcttttctcaaaattggagtcttttctttcaacgctgtgcagattgacttcgccagcagaacacgcgctgaggttaattaagggcccggccgtgacggccacgagcgaactcctgcaggtgtttaaactagggataacagggtaataggtctcacgcggcaaatcctaccacctcatttaaatagagtgaggttgatttgcggccgcacaacaaacgccggcgctctcctctcttaaggtagccggtgacacccttgggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggactttt

SEQ ID NO: 14 - DNA, 26 bp, synthetic construct
  source 1..26: mol_type = other DNA; note = A 26-nucleotide sequence corresponding to a thermal amplification primer referred to as SQ13805 which can be used to identify soybean event GM_CSM63770 DNA in a sample.
ccatattgaccatcatactcattgct

SEQ ID NO: 15 - DNA, 31 bp, synthetic construct
  source 1..31: mol_type = other DNA; note = A 31-nucleotide sequence corresponding to a thermal amplification primer referred to as SQ51400 which can be used to identify soybean event GM_CSM63770 DNA in a sample.
gtagtgcttctatttctgctagtggttataa

SEQ ID NO: 16 - DNA, 16 bp, synthetic construct
  source 1..16: mol_type = other DNA; note = A 16-nucleotide sequence corresponding to a probe referred to as PB4832 used to identify soybean event GM_CSM63770 DNA in a sample.
atccatgtagatttcc

SEQ ID NO: 17 - DNA, 20 bp, synthetic construct
  source 1..20: mol_type = other DNA; note = A 20-nucleotide sequence corresponding to a thermal amplification primer referred to as SQ549 used as an internal control for the event and zygosity assay for soybean event GM_CSM63770.
cggagatgctgtcgtgagaa

SEQ ID NO: 18 - DNA, 20 bp, synthetic construct
  source 1..20: mol_type = other DNA; note = A 20-nucleotide sequence corresponding to a thermal amplification primer referred to as SQ546 used as an internal control for the event and zygosity assay for soybean event GM_CSM63770.
ccctgtgatcgcagactcaa

SEQ ID NO: 19 - DNA, 28 bp, synthetic construct
  source 1..28: mol_type = other DNA; note = A 28-nucleotide sequence corresponding to a probe referred to as PB0004 used as an internal control for the event and zygosity assay for soybean event GM_CSM63770.
actctcgctagacacttccttccgcaaa

SEQ ID NO: 20 - DNA, 23 bp, synthetic construct
  source 1..23: mol_type = other DNA; note = A 25-nucleotide sequence corresponding to a thermal amplification primer referred to as GM_WT63770F used in the zygosity assay for soybean event GM_CSM63770.
ccccgttgtagtgtctgtttctc

SEQ ID NO: 21 - DNA, 24 bp, synthetic construct
  source 1..24: mol_type = other DNA; note = A 25-nucleotide sequence corresponding to a probe referred to as GM_WT63770PB used in the zygosity assay for soybean event GM_CSM63770.
cttgaattgtataaaattacaacc

SEQ ID NO: 22 - DNA, 27 bp, Glycine max
  source 1..27: mol_type = other DNA; note = A 27-nucleotide sequence corresponding to an originator guide RNA recognition site (OgRRS), OgRRS_5-1 comprised of a Cas12a protospacer adjacent motif (PAM) site operably linked to a guide-RNA hybridization site.
tttagagtttggagaataaagtacaca

SEQ ID NO: 23 - DNA, 27 bp, Glycine max
  source 1..27: mol_type = other DNA; note = A 27-nucleotide sequence corresponding to an OgRRS, OgRRS_5-2.
tttggcattggtcggagtctatgtttc

SEQ ID NO: 24 - DNA, 27 bp, Glycine max
  source 1..27: mol_type = other DNA; note = A 27-nucleotide sequence corresponding to an OgRRS, OgRRS_5-3.
tttggaggggagagataactacactaa

SEQ ID NO: 25 - DNA, 27 bp, Glycine max
  source 1..27: mol_type = other DNA; note = A 27-nucleotide sequence corresponding to an OgRRS, OgRRS_3-1.
tttggagagattcttggataaatgtat

SEQ ID NO: 26 - DNA, 27 bp, Glycine max
  source 1..27: mol_type = other DNA; note = A 27-nucleotide sequence corresponding to an OgRRS, OgRRS_In-1.
tttagggataatagctgtttgccaatc

SEQ ID NO: 27 - DNA, 27 bp, Glycine max
  source 1..27: mol_type = other DNA; note = A 27-nucleotide sequence corresponding to an OgRRS, OgRRS_In-2.
tttcgcaacaggcacagcgctgaggta

SEQ ID NO: 28 - DNA, 51 bp, synthetic construct
  source 1..51: mol_type = other DNA; note = A 51-nucleotide sequence corresponding to a guide-RNA (gRNA), gRNA_OgRRS_5-1.
gaatttctactaagtgtagatgagtttggagaataaagtacacattttttt

SEQ ID NO: 29 - DNA, 51 bp, synthetic construct
  source 1..51: mol_type = other DNA; note = A 51-nucleotide sequence corresponding to a gRNA, gRNA_OgRRS_5-2.
gaatttctactaagtgtagatgcattggtcggagtctatgtttcttttttt

SEQ ID NO: 30 - DNA, 51 bp, synthetic construct
  source 1..51: mol_type = other DNA; note = A 51-nucleotide sequence corresponding to a gRNA, gRNA_OgRRS_5-3.
gaatttctactaagtgtagatgaggggagagataactacactaattttttt

SEQ ID NO: 31 - DNA, 51 bp, synthetic construct
  source 1..51: mol_type = other DNA; note = A 51-nucleotide sequence corresponding to a gRNA, gRNA_OgRRS_3-1.
gaatttctactaagtgtagatgagagattcttggataaatgtatttttttt

SEQ ID NO: 32 - DNA, 51 bp, synthetic construct
  source 1..51: mol_type = other DNA; note = A 51-nucleotide sequence corresponding to a gRNA, gRNA_OgRRS_3-1.
gaatttctactaagtgtagatgggataatagctgtttgccaatcttttttt

SEQ ID NO: 33 - DNA, 51 bp, synthetic construct
  source 1..51: mol_type = other DNA; note = A 51-nucleotide sequence corresponding to a gRNA, gRNA_OgRRS_In-2.
gaatttctactaagtgtagatgcaacaggcacagcgctgaggtattttttt

SEQ ID NO: 34 - DNA, 3750 bp, synthetic construct
  source 1..3750: mol_type = other DNA; note = A sequence of a synthetic DNA coding sequence designed for expression in a plant cell encoding a nuclear targeted Cas12a CRISPR-associated protein.
atggcgggatctaagaagagaagaattaaacaagattcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcacggatctaagaagagaagaattaaacaagattga

SEQ ID NO: 35 - protein, 1249 aa, synthetic construct
  source 1..1249: note = Amino acid sequence of a nuclear targeted Cas12a CRISPR-associated protein.
MAGSKKRRIKQDSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGSKKRRIKQD

SEQ ID NO: 36 - DNA, 5000 bp, Glycine max
  source 1..5000: mol_type = genomic DNA; note = A 5,000-nucleotide sequence representing soybean genomic DNA that flanks the transgenic insert at the 5' end of the insert, the first 4,000 nucleotides of which are based on the genomic sequence of the Williams 82 cultivar.
aatgtggttctacagatcatattgctaaggattgcactggagattctacaatgaaacaaccacaagcaaaatacatcttaaaggatgataatgcccaacgtggtggagataatgcgaggtgtgtacttctgattgcatgtgcctgtcttttactgtctatctgattatgtctaagtgagtgatggaaatttcatggtgcagatatgaaatggtttttgatggagataactcacaaagtccaaggcgagatataaaacaccaatggcatgatggtgacctggttgaaagaaaagatcatagaagtcgtagagaacaggatatggtagactcaaacaacagagataggtatggtgatagaagcagaggacatgggggaaatggagacaacaaagcacggtctgaaagaggtgcaagagatttgggttcccatgctgacacgaaagacagagatagacacatgggaagacatggagatgatgattacaagcggaaagatgagtgtgactccagaaaaagagatgaagacgatggctacaagaggaaagatgagcaagactcaagaaaaagagatgaagacgatggctatagagaaagacgaactagtcgagatgataggagaaacatggattctagccatttggagagaaggaatgacagagattataggaagagacctgaagacagtggaaagcaagaaatgaagatggattctggacgaagaaaaggaagtccagttgatgaagattacaagaatggaagggatgatgaagattacaaacatagaagggaagaacgtgggcataaaagacaagatactaaagctgatgatgattacaagcgtagaagggaggatgtggattacaaacgcagaagggaggacggagattacagacatagaaggcaagaacgtggacataaaagacaagatattgtatctgatgattatcctcataggaggcatcatggtgataggagatgattattggtgtcacaatatccaaatgctaagcacagaacctttgaacggctatcttgtatgccctttttgcttcaatttttgtggtggagcaagacaatgatatactttagaacaaggtttttgaaatattctataagaacatatttaaacaaggtttttgtttgccactttgaacaaggtttacttggttaggccgtctctagagttattactagcaagctttgtaaaacctttttttttattatcgtcagcaacctctaacttgacaatgataacttgttgccatggattagcagttgttctcttctctgccatagtagctctctttctctcacatcttttgttccatctaattgacacaaacacaatagttttgactttatttgcagaactttgaggtcgttgctaatgcttcaccaatgttaaaacaaatcattaaccagtcgctaatatgtgagtttcttataatggtaacaatatgagcttccgtgcctacaaatatcttctaataaacacaaatggttcaattcctttatagttaaaccaaattaaaaaataatatttacttctgtagatgttttcttaatccaaatgcatcttatagaacgttataattaagttcactggaaaaaaggaaaaaagattactcccttacatataaaaacgttgctataacacatacgctgctaattaaacacacttgcatgtggacacaaccagcacttccaccaccatcattcattatcaaattgaaattcttgtttcaaacttattaattataatcttttcataatcctagccttcagaccattcttcatgagaaggacaatggagtggcttggagtaggagaatggccttccaccacctggaatcgatactttcgcaaaatagcagctgccaccatcttcatctgaacaaaggccaagtcctttcccaagcagattctaggtccagcattaaaagcaatgaatttgtaagctggtgcataaacaacctctcctctctcagatatccatctctctggcttgaactccaagcaatctttcccccatatctcttcacaccttcccattgcatacaaagaaaacaatatcattgtgtttccattcacacggtgaccacttggaagcgtgtcatctttaattgcctgcttccgctcaatagacacaggaggaaagagcctcaatgcttcacacaatgcaccatgcagataaactagctttttcacctcctcaatccctaaaaccccttcataattagcctcaaaattatcttttatctcttccagaatcttggcttccaccaaagggtgttttgtaacaagccaaaagaaccacgtgagagatgaagttatggtgtctcttccagccacaaaaagattaaactcatcatcccttagaaatctatcgtcatgtgcttgttcttgtcccatcaaagctgttaacaagtcaacatgagcttcacccatttcattttcgttgcacttgcttaaaagctctaccctcttggacgctatacgtgcatgtatgaatcgatcaagtgttttacatgcttctgtcatcttcttctcttgaccaatttgaagccatttctgcagcttccaaacacactttggcactgtatgtctgtagaatatggactcttctgcttcgttaaaagccttctcaattgcaacttcaggaaaatcaatggaaaggcaataaggatcatatccaagaacatcttgaaggtgaagcgattgaagacatcttgaagatctaccacccttccttgttgttgtacatgatccaatattggaagaaggcttgtttgatcaaggaatggaggccaggatggttctggagtacttccatgtctctgaatcggcagtgacaaccccatctccgaagggtttgaaaatctcgcgaaactcgggtcccttgatgtagttgtcaaagctcttgcagagcatgtgttgcacgttgagggagtcacaagtgatcaaatagtgcattttggtgaaccatggccctttgaattccccagatccaccttgttttttcagaatctcagtgcaaaaatcatggattcgccacatgttgaagaggactgatggtaacatgccaaggatggggtagtccctcatgagagggtgtttgcagcatcttctacgatggaacaagtataggaaggagaagagtgatgctgttatggctgcattgatcagaatcatgttggccatatcgtaaatttgagagagggttttaaatgtgaatgacttgccatctttgtgtgatatttgtcaatatatacagaaaattacagtgagatatatttgtctttttatatttttttaataataatttattaacacatgctaaaaaaaattacattattatttattattattttttaagtatgtgtgttaacaagttacacattattgtgtattttaaaatatttttagttatgacttatttatattaaaaaaaattaaagatgcataacaaaatcatatatatatatatatattcttattagttattagagagagtatatataactgatcttttctaatgtttagagtttttttccatttaaggaaaatattttttcagtattattcatgattttttttcttgttaattatgagatcaattttctttataatatattgtgtgtcagtttttattttctcaattctatattgcccatgtggatgacttgccgtatgtgtgtgatatttttttttcctcaacatattttgctcttaaatatttttcaagtttgcgcaaacacatgttataatttaatgaacatttctgtaaagtccagatcattatagttaattaaatatcattaaaaaaatacaaaaacgaaaaacaaattatactttcgattcacgcacatgaatcatgaacaatggttgaggctcgttgacattgcctgttagtgactgtgatgttatggatcgaacacttgaccctctgagatccaattttccacttgtctttcaaatgtgtctccttcacgatccaattataataacacaaaagagaatcaaaatgtttattggaaccagctctattcttagtttatttttattttggtgatatatttttaatttgttagaaataaatggtgtaacagttttaattagttttaatggtttgaacagttaattataatgaatgtatgagatgtgtgtactttattctccaaactctaaagtactgactttaaataaaaaatagttatcattgaaaaggtgttcatttttataactgttaacctataacttcttctatagttttgggtacaaaaactgttcacgtacgtacgtattcttataactaactgttaacctataacttgtttttctctcttcatcatattttaatttccgttttttcccctcatttctgtttctttgtatcgtagaaacatagactccgaccaatgccaaaatctttgatcttcattgttcatattctccacatctcaatcgattgttgcagaatcacagaaggagaaagaacataaagaaaagcttgcatttttgcctcaatatcatgttatttttgtttttggaggggagagataactacactaatctcttacgagatcagtgttaatattactacaaaaaagtgataatgatatttacatttttttaagtaaaacatatacatcttctaaaaaaaaagtaaaatatatacatatctctaatttatatttacatacgcatgcaatgttaggtatattcagcaatgcaacactattttcttttttctctataccgttttttctttattattgcatggatagtaggaaaaaataaataattaaaccaaaattaatatataagtcaatatattaaagataaattgttactagcaactttagcataataaaaatgggatatataattttagtgcagtcaaaattgtgataatatattaaatttagcataaaaagaaatgcaaataccccgttgtagtgtctgtttctcttagtacatatataataaaaccttgaatt

SEQ ID NO: 37 - DNA, 5000 bp, Glycine max
  source 1..5000: mol_type = genomic DNA; note = 5,000-nucleotide sequence representing soybean genomic DNA that flanks the transgenic insert at the 5' end of the insert, the last 4,000 nucleotides of which are based on the genomic sequence of the Williams 82 cultivar.
ccatagatgtgaggaagaatgtcaaaaatggaataaattttataaccactagcagaaatagaagcactacaatgtataactgcctttcccatgttttatttgtttcgtatgatctaagatgttaagttgacatgtttattaatcttgaagcactattaatttatttctacttatctgtacttttttttattcttaatttaataattttttcctcaatatattttaaaaatatatattaaactaaaattaatataaatgttaaagcatgataaaataaaatgttttgatatatcaaatttaacaaataaaaatacggacacacaaataagataacacatgtgtttttcattttgctaagtatatatattattgatcatatatcttatgattcatgatttgattcacgattccatacataggctttccgattcaactaccttgctggtaattatattgtctcacaccaagcatcaaaattatttgcaattacaaattaattgtttgctagttgttactgatgtcaatggtaaggaggcgttccgaggatctccttcgtttacttttgggctccatatcttccaaatgatgagtgatattgtcactattgatcaattttcactttaaaactgaatcattctgattaaaaaattaattcaaattaagtaactgattgaatttaaaattatatggaaaacaaagatatagattatgaatgagttgtcgctttgcttgttgttattgttaagtgaatttgggttcattgatttcaatgccgctggaatcaaatttgtaagtgaatttggagagattcttggataaatgtatcatatatttcatagtagcatgcatctcattctttgatttgggataaattagcaatttttgctattttacaggtccaatatgttcctgaacaattagaatcactaagggtacgtagtcctttgataactgccacatatatggagctcgaagatggactgagtagtagggaatggagaggggattaggcagagaccttggcaccaaaggaattaagcaacgatcgatttttgtatccaacagtttcaggcctcttattataatcatctcctacttgattttgttctttcctagttaattacttacgctaaatatcaaccctcttttgtgataccaaactataaaactacagaagctatttctttgttctccacgtaacatcttaaattcaatcagattttgtcaatgaaatctatataactcagctgattaaacagcatagcatacgtgagttattataaatcttttgatattctctttaattcttatagataaaaaaaatttaatcagttttttaattcgtcaaattgattcaatttaaagtaaaaaattgagcaatagtgacaatatgactaatcacaaaagtgcttgaaggagaaacaaagtacactaaaaaaatattgacattaattggatagaaaagaaagttgagccttactcaattatcatattaccctattacttccctcaacattgagcgaaaaactagaaaagcaagttctcaaacatactagtaagggatataaaaagccccttgacatcattcactatgaacaataattaaaaaaaaaaaatcactctccctggctcataatacatactgacagtggacaattttgaagaaccaggccatggtacttgaattttgaccatggacccttcatattcaaaatcaacttgtttgccatcaattctacaagttgttggtttctctgaggcatagaccctcatctccccagtgcctcttacaccaacttcaaccaaattctgagcctcatcaaaagccaaggactggactgctccaccagtgtttagcatattcaccaaaccaataggagcaaacttcactgacttgccacgcaaggcagtcactgaggaaaccgttataagctcaaaattgaacggctccaaggaaatctcttcactgtcatcagatggtgctgagaggatgagtttcttagcttggctgatataaggcgaaaagttgcaccacttcaatggaaattgggttctttccactgtcccattcaatgtcttggttgataccctgtgagaaaactcagcagcacatttgttggacctaatctcacggaaccaacctcctccctggcagttaaacaccccaataactccagtgtactgtatcaaaaagcaaattcacaataatgttaggttctagtctcagtagtagtattttacaccattttcattatttgaacatcaaataattaatactattgcatacaattatctctatgtctcagtattttatcttctttatatagtgtaaaatgcattttccaattgacagtggtaaatgttcaatttgcttttaagcaatatcattatagaaacatagcaaatatctggaagtaccttggtaaaagctctattgctcattttcttcatcgttaacatttaaggtcctttgctgataaacttctccctaagagaaagaaattcaggaagtaaaatgaattggatttttcctataaacaaaaattaacttttataaaaattaaatatataggttgattttagcacatgggagaagtttaaatcattttaactttttattttatttttttctcaggtgcttatgtgtaaggtttttcaaagagaatctaaagggattggaagagaacgtatgctgggtccgattggagcatgaggaattgggagaggatgacgtggccttttacttttagggttgagttgctttgtcacgggtgaaggcttgttcaagcttggagccattagtgagactcaagagttagatacaagaaataatgtatgtgtgagtaagaatggggagagtttacaagttttaagaatttaatatattatttttattattctaatcttttttataaaaaaaaaactctcttatttcataaatatatacaaataaatattttcataaaaaaatgattagttgagatccaacaaactataatttatgacataaattagacttaaaatatatgtattttttacaaaaaaataactatgtttgagtaaggatttttataaccaacaaaacactcccttcaaatctatagcttttgagaatacggaggagtgtactaatttggagacatgcaattaaatttccacatacttcacatttgtttactaactataataattattctgtaaaacattattcattgtacaaggattctatgcatgcttttgtttcttcatcttttaaaaaaaggaagtcaaggattctatattatgtgaacaagagaaaggttcgtcttactgtttccgcaataacttatgataataattaattttgatgttgaaatagaacaaaacatatgatctagattttcataaaaaaaacaaaaaataatgcatgtggctttgaggatgagagggttagtttgaagccgttagtgtacgttgagaacagttgttgagtcaatagtcaagaaactcaatgtagggtaggggcacatggcattccaatatttggatgagataaaacagttatgttatgtatattgcaagtgccggtgggaaaaggttacatgcacagtgagtgacattgatcatttgaaaagatgacaatgacaataacaattgacagttaaaaccttcaatagcgtgttccctttcttgcattctattgcgagtaacgtggcatgaattgttgaacgtatcaactaaagagccttactaaatgacaaaattaatatatgttatagtagtataaggtaatggttaattagcttaaattgatggacgatgacccactaagaaataaaggtgagttgttaattcttcactgaccccaactcaaagatcacggacacttctagagttctagtactttaaagattgttgggaccttttgtttttcggccaacaagattgttagattgttgacaccataaagatgggctccaccatctagcaactccatatcctctcctttttgttgttctcttcttctcaactcaattcaattcttcttcttgaaattagctttcctattttgagttttgaatttgtttctagaatagacattctgtaagggtatgtatactcttacagaattatcctttgggaaggaaaaatcttacagaacaccttttctgtaagtaaaaatagtgcttctaaaacgaccattttggaatataaaaatatgcttccggaaaggctatttaggattgtaatttttactttcaaaatgattattccataagctaaaatagtactttctagaatgtccttttcaaaagatacaaaatacgcttctgaaaaggttactccgaattgcagttgttgctttgggaatgagcttttcacaagtataataatgattttccgaaaccccctttatggaatttacaaaatacactttcgaaaatgttgctctggatttttaaaaaattgtatccaaaatggtttttccctaaacaaaaatgcttctagaacaaccttttacttatagaaagaccattctagaaacattttttgcttatagaaagatcattccgaaagtattttggcttatgaaaaggccattttagaattttttttatagttaattaaatttgataaagggagaaggagtcacttaccttcatggaagaaggaaagaggatgagggagtgcaagaatgagggggtggggcaaggaagagtcgttgcgggagggaggggtgtcgtcaatgtgggtgtgtcatcaaagaaaggatgtcgtcgttgtgggtgtgccgtcgaagaaggaatgtcatcgcatgttgagagaaaggaaaaaggaggtgttaggattgagagatacgaagtgaaaatttcaataaaagtattattatatttttactgaataatgagagatgcagcatacacataagaaagtgcatgattcaagtttcttaatttcttcaaagcacaagcaaacgaagcctaatagagaaccatggtcttaatttgtttttctttaag

SEQ ID NO: 38 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
aatgtggttctacagatcatattgctaaggattgcactggagattctaca

SEQ ID NO: 39 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
atgaaacaaccacaagcaaaatacatcttaaaggatgataatgcccaacg

SEQ ID NO: 40 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tggtggagataatgcgaggtgtgtacttctgattgcatgtgcctgtcttt

SEQ ID NO: 41 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tactgtctatctgattatgtctaagtgagtgatggaaatttcatggtgca

SEQ ID NO: 42 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gatatgaaatggtttttgatggagataactcacaaagtccaaggcgagat

SEQ ID NO: 43 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ataaaacaccaatggcatgatggtgacctggttgaaagaaaagatcatag

SEQ ID NO: 44 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
aagtcgtagagaacaggatatggtagactcaaacaacagagataggtatg

SEQ ID NO: 45 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gtgatagaagcagaggacatgggggaaatggagacaacaaagcacggtct

SEQ ID NO: 46 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gaaagaggtgcaagagatttgggttcccatgctgacacgaaagacagaga

SEQ ID NO: 47 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tagacacatgggaagacatggagatgatgattacaagcggaaagatgagt

SEQ ID NO: 48 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gtgactccagaaaaagagatgaagacgatggctacaagaggaaagatgag

SEQ ID NO: 49 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
caagactcaagaaaaagagatgaagacgatggctatagagaaagacgaac

SEQ ID NO: 50 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tagtcgagatgataggagaaacatggattctagccatttggagagaagga

SEQ ID NO: 51 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
atgacagagattataggaagagacctgaagacagtggaaagcaagaaatg

SEQ ID NO: 52 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
aagatggattctggacgaagaaaaggaagtccagttgatgaagattacaa

SEQ ID NO: 53 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gaatggaagggatgatgaagattacaaacatagaagggaagaacgtgggc

SEQ ID NO: 54 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ataaaagacaagatactaaagctgatgatgattacaagcgtagaagggag

SEQ ID NO: 55 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gatgtggattacaaacgcagaagggaggacggagattacagacatagaag

SEQ ID NO: 56 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gcaagaacgtggacataaaagacaagatattgtatctgatgattatcctc

SEQ ID NO: 57 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ataggaggcatcatggtgataggagatgattattggtgtcacaatatcca

SEQ ID NO: 58 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
aatgctaagcacagaacctttgaacggctatcttgtatgccctttttgct

SEQ ID NO: 59 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tcaatttttgtggtggagcaagacaatgatatactttagaacaaggtttt

SEQ ID NO: 60 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tgaaatattctataagaacatatttaaacaaggtttttgtttgccacttt

SEQ ID NO: 61 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gaacaaggtttacttggttaggccgtctctagagttattactagcaagct

SEQ ID NO: 62 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ttgtaaaacctttttttttattatcgtcagcaacctctaacttgacaatg

SEQ ID NO: 63 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ataacttgttgccatggattagcagttgttctcttctctgccatagtagc

SEQ ID NO: 64 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tctctttctctcacatcttttgttccatctaattgacacaaacacaatag

SEQ ID NO: 65 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ttttgactttatttgcagaactttgaggtcgttgctaatgcttcaccaat

SEQ ID NO: 66 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gttaaaacaaatcattaaccagtcgctaatatgtgagtttcttataatgg

SEQ ID NO: 67 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
taacaatatgagcttccgtgcctacaaatatcttctaataaacacaaatg

SEQ ID NO: 68 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gttcaattcctttatagttaaaccaaattaaaaaataatatttacttctg

SEQ ID NO: 69 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tagatgttttcttaatccaaatgcatcttatagaacgttataattaagtt

SEQ ID NO: 70 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
cactggaaaaaaggaaaaaagattactcccttacatataaaaacgttgct

SEQ ID NO: 71 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ataacacatacgctgctaattaaacacacttgcatgtggacacaaccagc

SEQ ID NO: 72 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
acttccaccaccatcattcattatcaaattgaaattcttgtttcaaactt

SEQ ID NO: 73 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
attaattataatcttttcataatcctagccttcagaccattcttcatgag

SEQ ID NO: 74 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
aaggacaatggagtggcttggagtaggagaatggccttccaccacctgga

SEQ ID NO: 75 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
atcgatactttcgcaaaatagcagctgccaccatcttcatctgaacaaag

SEQ ID NO: 76 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gccaagtcctttcccaagcagattctaggtccagcattaaaagcaatgaa

SEQ ID NO: 77 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tttgtaagctggtgcataaacaacctctcctctctcagatatccatctct

SEQ ID NO: 78 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ctggcttgaactccaagcaatctttcccccatatctcttcacaccttccc

SEQ ID NO: 79 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
attgcatacaaagaaaacaatatcattgtgtttccattcacacggtgacc

SEQ ID NO: 80 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
acttggaagcgtgtcatctttaattgcctgcttccgctcaatagacacag

SEQ ID NO: 81 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gaggaaagagcctcaatgcttcacacaatgcaccatgcagataaactagc

SEQ ID NO: 82 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tttttcacctcctcaatccctaaaaccccttcataattagcctcaaaatt

SEQ ID NO: 83 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
atcttttatctcttccagaatcttggcttccaccaaagggtgttttgtaa

SEQ ID NO: 84 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
caagccaaaagaaccacgtgagagatgaagttatggtgtctcttccagcc

SEQ ID NO: 85 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
acaaaaagattaaactcatcatcccttagaaatctatcgtcatgtgcttg

SEQ ID NO: 86 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ttcttgtcccatcaaagctgttaacaagtcaacatgagcttcacccattt

SEQ ID NO: 87 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
cattttcgttgcacttgcttaaaagctctaccctcttggacgctatacgt

SEQ ID NO: 88 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gcatgtatgaatcgatcaagtgttttacatgcttctgtcatcttcttctc

SEQ ID NO: 89 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ttgaccaatttgaagccatttctgcagcttccaaacacactttggcactg

SEQ ID NO: 90 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tatgtctgtagaatatggactcttctgcttcgttaaaagccttctcaatt

SEQ ID NO: 91 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gcaacttcaggaaaatcaatggaaaggcaataaggatcatatccaagaac

SEQ ID NO: 92 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
atcttgaaggtgaagcgattgaagacatcttgaagatctaccacccttcc

SEQ ID NO: 93 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ttgttgttgtacatgatccaatattggaagaaggcttgtttgatcaagga

SEQ ID NO: 94 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
atggaggccaggatggttctggagtacttccatgtctctgaatcggcagt

SEQ ID NO: 95 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gacaaccccatctccgaagggtttgaaaatctcgcgaaactcgggtccct

SEQ ID NO: 96 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tgatgtagttgtcaaagctcttgcagagcatgtgttgcacgttgagggag

SEQ ID NO: 97 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tcacaagtgatcaaatagtgcattttggtgaaccatggccctttgaattc

SEQ ID NO: 98 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
cccagatccaccttgttttttcagaatctcagtgcaaaaatcatggattc

SEQ ID NO: 99 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gccacatgttgaagaggactgatggtaacatgccaaggatggggtagtcc

SEQ ID NO: 100 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ctcatgagagggtgtttgcagcatcttctacgatggaacaagtataggaa

SEQ ID NO: 101 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ggagaagagtgatgctgttatggctgcattgatcagaatcatgttggcca

SEQ ID NO: 102 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tatcgtaaatttgagagagggttttaaatgtgaatgacttgccatctttg

SEQ ID NO: 103 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tgtgatatttgtcaatatatacagaaaattacagtgagatatatttgtct

SEQ ID NO: 104 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ttttatatttttttaataataatttattaacacatgctaaaaaaaattac

SEQ ID NO: 105 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
attattatttattattattttttaagtatgtgtgttaacaagttacacat

SEQ ID NO: 106 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tattgtgtattttaaaatatttttagttatgacttatttatattaaaaaa

SEQ ID NO: 107 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
aattaaagatgcataacaaaatcatatatatatatatatattcttattag

SEQ ID NO: 108 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ttattagagagagtatatataactgatcttttctaatgtttagagttttt

SEQ ID NO: 109 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ttccatttaaggaaaatattttttcagtattattcatgattttttttctt

SEQ ID NO: 110 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gttaattatgagatcaattttctttataatatattgtgtgtcagttttta

SEQ ID NO: 111 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
ttttctcaattctatattgcccatgtggatgacttgccgtatgtgtgtga

SEQ ID NO: 112 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
tatttttttttcctcaacatattttgctcttaaatatttttcaagtttgc

SEQ ID NO: 113 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gcaaacacatgttataatttaatgaacatttctgtaaagtccagatcatt

SEQ ID NO: 114 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
atagttaattaaatatcattaaaaaaatacaaaaacgaaaaacaaattat

SEQ ID NO: 115 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
actttcgattcacgcacatgaatcatgaacaatggttgaggctcgttgac

SEQ ID NO: 116 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
attgcctgttagtgactgtgatgttatggatcgaacacttgaccctctga

SEQ ID NO: 117 - DNA, 50 bp, Glycine max
  source 1..50: mol_type = genomic DNA; note = Additional 50-nucleotide sequence in the 5' flank of event GM_63770 based on the genomic sequence of the Williams 82 cultivar.
gatccaattttccacttgtctttcaaatgtgtctccttcacgatccaatt

SEQ ID NO: 118 - DNA, 50 bp, Glycine max
  source 1..50: mol_type = genomic DNA; note = A 50-nucleotide sequences in the 5' flank genomic sequence of event GM_CSM63770.
ataataacacaaaagagaatcaaaatgtttattggaaccagctctattct

SEQ ID NO: 119 - DNA, 50 bp, Glycine max
  source 1..50: mol_type = genomic DNA; note = A 50-nucleotide sequences in the 5' flank genomic sequence of event GM_CSM63770.
tagtttatttttattttggtgatatatttttaatttgttagaaataaatg

SEQ ID NO: 120 - DNA, 50 bp, Glycine max
  source 1..50: mol_type = genomic DNA; note = A 50-nucleotide sequences in the 5' flank genomic sequence of event GM_CSM63770.
gtgtaacagttttaattagttttaatggtttgaacagttaattataatga

SEQ ID NO: 121 - DNA, 50 bp, Glycine max
  source 1..50: mol_type = genomic DNA; note = A 50-nucleotide sequences in the 5' flank genomic sequence of event GM_CSM63770.
atgtatgagatgtgtgtactttattctccaaactctaaagtactgacttt

SEQ ID NO: 122 - DNA, 50 bp, Glycine max
  source 1..50: mol_type = genomic DNA; note = A 50-nucleotide sequences in the 5' flank genomic sequence of event GM_CSM63770.
aaataaaaaatagttatcattgaaaaggtgttcatttttataactgttaa

SEQ ID NO: 123 - DNA, 50 bp, Glycine max
  source 1..50: mol_type = genomic DNA; note = A 50-nucleotide sequences in the 5' flank genomic sequence of event GM_CSM63770.
cctataacttcttctatagttttgggtacaaaaactgttcacgtacgtac

SEQ ID NO: 124 - DNA, 50 bp, Glycine max
  source 1..50: mol_type = genomic DNA; note = A 50-nucleotide sequences in the 5' flank genomic sequence of event GM_CSM63770.
gtattcttataactaactgttaacctataacttgtttttctctcttcatc

SEQ ID NO: 125 - DNA, 50 bp, Glycine max
  source 1..50: mol_type = genomic DNA; note = A 50-nucleotide sequences in the 5' flank genomic sequence of event GM_CSM63770.
atattttaatttccgttttttcccctcatttctgtttctttgtatcgtag

SEQ ID NO: 126 - DNA, 50 bp, Glycine max
  source 1..50: mol_type = genomic DNA; note = A 50-nucleotide sequences in the 5' flank genomic sequence of event GM_CSM63770.
aaacatagactccgaccaatgccaaaatctttgatcttcattgttcatat

SEQ ID NO: 127 - DNA, 50 bp, Glycine max
  source 1..50: mol_type = genomic DNA; note = A 50-nucleotide sequences in the 5' flank genomic sequence of event GM_CSM63770.
tctccacatctcaatcgattgttgcagaatcacagaaggagaaagaacat

SEQ ID NO: 128 - DNA, 50 bp, Glycine max
  source 1..50: mol_type = genomic DNA; note = A 50-nucleotide sequences in the 5' flank genomic sequence of event GM_CSM63770.
aaagaaaagcttgcatttttgcctcaatatcatgttatttttgtttttgg

SEQ ID NO: 129 - DNA, 50 bp, Glycine max
  source 1..50: mol_type = genomic DNA; note = A 50-nucleotide sequences in the 5' flank genomic sequence of event GM_CSM63770.
aggggagagataactacactaatctcttacgagatcagtgttaatattac

SEQ ID NO: 130 - DNA, 50 bp, Glycine max
  source 1..50: mol_type = genomic DNA; note = A 50-nucleotide sequences in the 5' flank genomic sequence of event GM_CSM63770.
tacaaaaaagtgataatgatatttacatttttttaagtaaaacatataca

SEQ ID NO: 131 - DNA, 50 bp, Glycine max
  source 1..50: mol_type = genomic DNA; note = A 50-nucleotide sequences in the 5' flank genomic sequence of event GM_CSM63770.
tcttctaaaaaaaaagtaaaatatatacatatctctaatttatatttaca

SEQ ID NO: 132 - DNA, 50 bp, Glycine max
  source 1..50: mol_type = genomic DNA; note = A 50-nucleotide sequences in the 5' flank genomic sequence of event GM_CSM63770.
tacgcatgcaatgttaggtatattcagcaatgcaacactattttcttttt

SEQ ID NO: 133 - DNA, 50 bp, Glycine max
  source 1..50: mol_type = genomic DNA; note = A 50-nucleotide sequences in the 5' flank genomic sequence of event GM_CSM63770.
tctctataccgttttttctttattattgcatggatagtaggaaaaaataa

SEQ ID NO: 134 - DNA, 50 bp, Glycine max
  source 1..50: mol_type = genomic DNA; note = A 50-nucleotide sequences in the 5' flank genomic sequence of event GM_CSM63770.
ataattaaaccaaaattaatatataagtcaatatattaaagataaattgt

SEQ ID NO: 135 - DNA, 50 bp, Glycine max
  source 1..50: mol_type = genomic DNA; note = A 50-nucleotide sequences in the 5' flank genomic sequence of event GM_CSM63770.
tactagcaactttagcataataaaaatgggatatataattttagtgcagt

SEQ ID NO: 136 - DNA, 50 bp, Glycine max
  source 1..50: mol_type = genomic DNA; note = A 50-nucleotide sequences in the 5' flank genomic sequence of event GM_CSM63770.
caaaattgtgataatatattaaatttagcataaaaagaaatgcaaatacc

SEQ ID NO: 137 - DNA, 50 bp, Glycine max
  source 1..50: mol_type = genomic DNA; note = A 50-nucleotide sequences in the 5' flank genomic sequence of event GM_CSM63770.
ccgttgtagtgtctgtttctcttagtacatatataataaaaccttgaatt

SEQ ID NO: 138 - DNA, 50 bp, Glycine max
  source 1..50: mol_type = genomic DNA; note = A 50-nucleotide sequences in the 3' flank genomic sequence of event GM_CSM63770.
ccatagatgtgaggaagaatgtcaaaaatggaataaattttataaccact

SEQ ID NO: 139 - DNA, 50 bp, Glycine max
  source 1..50: mol_type = genomic DNA; note = A 50-nucleotide sequences in the 3' flank genomic sequence of event GM_CSM63770.
agcagaaatagaagcactacaatgtataactgcctttcccatgttttatt

SEQ ID NO: 140 - DNA, 50 bp, Glycine max
  source 1..50: mol_type = genomic DNA; note = A 50-nucleotide sequences in the 3' flank genomic sequence of event GM_CSM63770.
tgtttcgtatgatctaagatgttaagttgacatgtttattaatcttgaag

SEQ ID NO: 141 - DNA, 50 bp, Glycine max
  source 1..50: mol_type = genomic DNA; note = A 50-nucleotide sequences in the 3' flank genomic sequence of event GM_CSM63770.
cactattaatttatttctacttatctgtacttttttttattcttaattta

SEQ ID NO: 142 - DNA, 50 bp, Glycine max
  source 1..50: mol_type = genomic DNA; note = A 50-nucleotide sequences in the 3' flank genomic sequence of event GM_CSM63770.
ataattttttcctcaatatattttaaaaatatatattaaactaaaattaa

SEQ ID NO: 143 - DNA, 50 bp, Glycine max
  source 1..50: mol_type = genomic DNA; note = A 50-nucleotide sequences in the 3' flank genomic sequence of event GM_CSM63770.
tataaatgttaaagcatgataaaataaaatgttttgatatatcaaattta

SEQ ID NO: 144 - DNA, 50 bp, Glycine max
  source 1..50: mol_type = genomic DNA; note = A 50-nucleotide sequences in the 3' flank genomic sequence of event GM_CSM63770.
acaaataaaaatacggacacacaaataagataacacatgtgtttttcatt

SEQ ID NO: 145 - DNA, 50 bp, Glycine max
  source 1..50: mol_type = genomic DNA; note = A 50-nucleotide sequences in the 3' flank genomic sequence of event GM_CSM63770.
ttgctaagtatatatattattgatcatatatcttatgattcatgatttga

SEQ ID NO: 146 - DNA, 50 bp, Glycine max
  source 1..50: mol_type = genomic DNA; note = A 50-nucleotide sequences in the 3' flank genomic sequence of event GM_CSM63770.
ttcacgattccatacataggctttccgattcaactaccttgctggtaatt

SEQ ID NO: 147 - DNA, 50 bp, Glycine max
  source 1..50: mol_type = genomic DNA; note = A 50-nucleotide sequences in the 3' flank genomic sequence of event GM_CSM63770.
atattgtctcacaccaagcatcaaaattatttgcaattacaaattaattg

SEQ ID NO: 148 - DNA, 50 bp, Glycine max
  source 1..50: mol_type = genomic DNA; note = A 50-nucleotide sequences in the 3' flank genomic sequence of event GM_CSM63770.
tttgctagttgttactgatgtcaatggtaaggaggcgttccgaggatctc

SEQ ID NO: 149 - DNA, 50 bp, Glycine max
  source 1..50: mol_type = genomic DNA; note = A 50-nucleotide sequences in the 3' flank genomic sequence of event GM_CSM63770.
cttcgtttacttttgggctccatatcttccaaatgatgagtgatattgtc

SEQ ID NO: 150 - DNA, 50 bp, Glycine max
  source 1..50: mol_type = genomic DNA; note = A 50-nucleotide sequences in the 3' flank genomic sequence of event GM_CSM63770.
actattgatcaattttcactttaaaactgaatcattctgattaaaaaatt

SEQ ID NO: 151 - DNA, 50 bp, Glycine max
  source 1..50: mol_type = genomic DNA; note = A 50-nucleotide sequences in the 3' flank genomic sequence of event GM_CSM63770.
aattcaaattaagtaactgattgaatttaaaattatatggaaaacaaaga

SEQ ID NO: 152 - DNA, 50 bp, Glycine max
  source 1..50: mol_type = genomic DNA; note = A 50-nucleotide sequences in the 3' flank genomic sequence of event GM_CSM63770.
tatagattatgaatgagttgtcgctttgcttgttgttattgttaagtgaa

SEQ ID NO: 153 - DNA, 50 bp, Glycine max
  source 1..50: mol_type = genomic DNA; note = A 50-nucleotide sequences in the 3' flank genomic sequence of event GM_CSM63770.
tttgggttcattgatttcaatgccgctggaatcaaatttgtaagtgaatt

SEQ ID NO: 154 - DNA, 50 bp, Glycine max
  source 1..50: mol_type = genomic DNA; note = A 50-nucleotide sequences in the 3' flank genomic sequence of event GM_CSM63770.
tggagagattcttggataaatgtatcatatatttcatagtagcatgcatc

SEQ ID NO: 155 - DNA, 50 bp, Glycine max
  source 1..50: mol_type = genomic DNA; note = A 50-nucleotide sequences in the 3' flank genomic sequence of event GM_CSM63770.
tcattctttgatttgggataaattagcaatttttgctattttacaggtcc

SEQ ID NO: 156 - DNA, 50 bp, Glycine max
  source 1..50: mol_type = genomic DNA; note = A 50-nucleotide sequences in the 3' flank genomic sequence of event GM_CSM63770.
aatatgttcctgaacaattagaatcactaagggtacgtagtcctttgata

SEQ ID NO: 157 - DNA, 50 bp, Glycine max
  source 1..50: mol_type = genomic DNA; note = A 50-nucleotide sequences in the 3' flank genomic sequence of event GM_CSM63770.
actgccacatatatggagctcgaagatggactgagtagtagggaatggag

SEQ ID NO: 158 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
aggggattaggcagagaccttggcaccaaaggaattaagcaacgatcgat

SEQ ID NO: 159 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ttttgtatccaacagtttcaggcctcttattataatcatctcctacttga

SEQ ID NO: 160 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ttttgttctttcctagttaattacttacgctaaatatcaaccctcttttg

SEQ ID NO: 161 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tgataccaaactataaaactacagaagctatttctttgttctccacgtaa

SEQ ID NO: 162 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
catcttaaattcaatcagattttgtcaatgaaatctatataactcagctg

SEQ ID NO: 163 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
attaaacagcatagcatacgtgagttattataaatcttttgatattctct

SEQ ID NO: 164 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ttaattcttatagataaaaaaaatttaatcagttttttaattcgtcaaat

SEQ ID NO: 165 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tgattcaatttaaagtaaaaaattgagcaatagtgacaatatgactaatc

SEQ ID NO: 166 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
acaaaagtgcttgaaggagaaacaaagtacactaaaaaaatattgacatt

SEQ ID NO: 167 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
aattggatagaaaagaaagttgagccttactcaattatcatattacccta

SEQ ID NO: 168 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ttacttccctcaacattgagcgaaaaactagaaaagcaagttctcaaaca

SEQ ID NO: 169 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tactagtaagggatataaaaagccccttgacatcattcactatgaacaat

SEQ ID NO: 170 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
aattaaaaaaaaaaaatcactctccctggctcataatacatactgacagt

SEQ ID NO: 171 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ggacaattttgaagaaccaggccatggtacttgaattttgaccatggacc

SEQ ID NO: 172 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
cttcatattcaaaatcaacttgtttgccatcaattctacaagttgttggt

SEQ ID NO: 173 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ttctctgaggcatagaccctcatctccccagtgcctcttacaccaacttc

SEQ ID NO: 174 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
aaccaaattctgagcctcatcaaaagccaaggactggactgctccaccag

SEQ ID NO: 175 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tgtttagcatattcaccaaaccaataggagcaaacttcactgacttgcca

SEQ ID NO: 176 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
cgcaaggcagtcactgaggaaaccgttataagctcaaaattgaacggctc

SEQ ID NO: 177 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
caaggaaatctcttcactgtcatcagatggtgctgagaggatgagtttct

SEQ ID NO: 178 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tagcttggctgatataaggcgaaaagttgcaccacttcaatggaaattgg

SEQ ID NO: 179 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gttctttccactgtcccattcaatgtcttggttgataccctgtgagaaaa

SEQ ID NO: 180 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ctcagcagcacatttgttggacctaatctcacggaaccaacctcctccct

SEQ ID NO: 181 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ggcagttaaacaccccaataactccagtgtactgtatcaaaaagcaaatt

SEQ ID NO: 182 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
cacaataatgttaggttctagtctcagtagtagtattttacaccattttc

SEQ ID NO: 183 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
attatttgaacatcaaataattaatactattgcatacaattatctctatg

SEQ ID NO: 184 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tctcagtattttatcttctttatatagtgtaaaatgcattttccaattga

SEQ ID NO: 185 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
cagtggtaaatgttcaatttgcttttaagcaatatcattatagaaacata

SEQ ID NO: 186 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gcaaatatctggaagtaccttggtaaaagctctattgctcattttcttca

SEQ ID NO: 187 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tcgttaacatttaaggtcctttgctgataaacttctccctaagagaaaga

SEQ ID NO: 188 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
aattcaggaagtaaaatgaattggatttttcctataaacaaaaattaact

SEQ ID NO: 189 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tttataaaaattaaatatataggttgattttagcacatgggagaagttta

SEQ ID NO: 190 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
aatcattttaactttttattttatttttttctcaggtgcttatgtgtaag

SEQ ID NO: 191 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gtttttcaaagagaatctaaagggattggaagagaacgtatgctgggtcc

SEQ ID NO: 192 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gattggagcatgaggaattgggagaggatgacgtggccttttacttttag

SEQ ID NO: 193 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ggttgagttgctttgtcacgggtgaaggcttgttcaagcttggagccatt

SEQ ID NO: 194 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
agtgagactcaagagttagatacaagaaataatgtatgtgtgagtaagaa

SEQ ID NO: 195 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tggggagagtttacaagttttaagaatttaatatattatttttattattc

SEQ ID NO: 196 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
taatcttttttataaaaaaaaaactctcttatttcataaatatatacaaa

SEQ ID NO: 197 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
taaatattttcataaaaaaatgattagttgagatccaacaaactataatt

SEQ ID NO: 198 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tatgacataaattagacttaaaatatatgtattttttacaaaaaaataac

SEQ ID NO: 199 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tatgtttgagtaaggatttttataaccaacaaaacactcccttcaaatct

SEQ ID NO: 200 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
atagcttttgagaatacggaggagtgtactaatttggagacatgcaatta

SEQ ID NO: 201 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
aatttccacatacttcacatttgtttactaactataataattattctgta

SEQ ID NO: 202 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
aaacattattcattgtacaaggattctatgcatgcttttgtttcttcatc

SEQ ID NO: 203 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ttttaaaaaaaggaagtcaaggattctatattatgtgaacaagagaaagg

SEQ ID NO: 204 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ttcgtcttactgtttccgcaataacttatgataataattaattttgatgt

SEQ ID NO: 205 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tgaaatagaacaaaacatatgatctagattttcataaaaaaaacaaaaaa

SEQ ID NO: 206 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
taatgcatgtggctttgaggatgagagggttagtttgaagccgttagtgt

SEQ ID NO: 207 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
acgttgagaacagttgttgagtcaatagtcaagaaactcaatgtagggta

SEQ ID NO: 208 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ggggcacatggcattccaatatttggatgagataaaacagttatgttatg

SEQ ID NO: 209 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tatattgcaagtgccggtgggaaaaggttacatgcacagtgagtgacatt

SEQ ID NO: 210 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gatcatttgaaaagatgacaatgacaataacaattgacagttaaaacctt

SEQ ID NO: 211 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
caatagcgtgttccctttcttgcattctattgcgagtaacgtggcatgaa

SEQ ID NO: 212 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ttgttgaacgtatcaactaaagagccttactaaatgacaaaattaatata

SEQ ID NO: 213 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tgttatagtagtataaggtaatggttaattagcttaaattgatggacgat

SEQ ID NO: 214 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gacccactaagaaataaaggtgagttgttaattcttcactgaccccaact

SEQ ID NO: 215 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
caaagatcacggacacttctagagttctagtactttaaagattgttggga

SEQ ID NO: 216 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ccttttgtttttcggccaacaagattgttagattgttgacaccataaaga

SEQ ID NO: 217 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tgggctccaccatctagcaactccatatcctctcctttttgttgttctct

SEQ ID NO: 218 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tcttctcaactcaattcaattcttcttcttgaaattagctttcctatttt

SEQ ID NO: 219 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gagttttgaatttgtttctagaatagacattctgtaagggtatgtatact

SEQ ID NO: 220 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
cttacagaattatcctttgggaaggaaaaatcttacagaacaccttttct

SEQ ID NO: 221 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gtaagtaaaaatagtgcttctaaaacgaccattttggaatataaaaatat

SEQ ID NO: 222 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gcttccggaaaggctatttaggattgtaatttttactttcaaaatgatta

SEQ ID NO: 223 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ttccataagctaaaatagtactttctagaatgtccttttcaaaagataca

SEQ ID NO: 224 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
aaatacgcttctgaaaaggttactccgaattgcagttgttgctttgggaa

SEQ ID NO: 225 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tgagcttttcacaagtataataatgattttccgaaaccccctttatggaa

SEQ ID NO: 226 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
tttacaaaatacactttcgaaaatgttgctctggatttttaaaaaattgt

SEQ ID NO: 227 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
atccaaaatggtttttccctaaacaaaaatgcttctagaacaacctttta

SEQ ID NO: 228 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
cttatagaaagaccattctagaaacattttttgcttatagaaagatcatt

SEQ ID NO: 229 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ccgaaagtattttggcttatgaaaaggccattttagaattttttttatag

SEQ ID NO: 230 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ttaattaaatttgataaagggagaaggagtcacttaccttcatggaagaa

SEQ ID NO: 231 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ggaaagaggatgagggagtgcaagaatgagggggtggggcaaggaagagt

SEQ ID NO: 232 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
cgttgcgggagggaggggtgtcgtcaatgtgggtgtgtcatcaaagaaag

SEQ ID NO: 233 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gatgtcgtcgttgtgggtgtgccgtcgaagaaggaatgtcatcgcatgtt

SEQ ID NO: 234 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gagagaaaggaaaaaggaggtgttaggattgagagatacgaagtgaaaat

SEQ ID NO: 235 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
ttcaataaaagtattattatatttttactgaataatgagagatgcagcat

SEQ ID NO: 236 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
acacataagaaagtgcatgattcaagtttcttaatttcttcaaagcacaa

SEQ ID NO: 237 - DNA, 50 bp, Glycine max
  source 1..50: mol_type = genomic DNA; note = Additional 50-nucleotide sequence in the 3' flank of event GM_63770 based on the genomic sequence of the Williams 82 cultivar.
gcaaacgaagcctaatagagaaccatggtcttaatttgtttttctttaag

(19) United States
(12) Patent Application Publication
(10) Pub. No.: US 2024/0035100 A1
(43) Pub. Date: Feb. 1, 2024

(54) SOYBEAN TRANSGENIC EVENT GM_CSM63770 AND METHODS FOR DETECTION AND USES THEREOF

(71) Applicant: Monsanto Technology LLC, St. Louis, MO (US)

(72) Inventors: Sarah L. Brown, Weldon Spring, MO (US); Karen K. Gabbert, St. Louis, MO (US); Anilkumar Gowda, Chesterfield, MO (US); Ted C. MacRae, Wildwood, MO (US); Lisa G. Ruschke, St. Louis, MO (US); Linda Rymarquis, High Ridge, MO (US); Xiaohong Shi, St. Charles, MO (US)

(21) Appl. No.: 18/341,611

(22) Filed: Jun. 26, 2023

Related U.S. Application Data
(60) Provisional application No. 63/355,947, filed on Jun. 27, 2022.

(51) Int. Cl. C12Q 1/6895; C12N 15/82

(52) U.S. Cl. CPC C12Q 1/6895; C12N 15/8286; C12Q 2600/156

(57) ABSTRACT

The invention provides a transgenic soybean event GM_CSM63770, plants, plant cells, seed, plant parts, progeny plants, commodity products comprising event GM_CSM63770, polynucleotides specific for defining and detecting event GM_CSM63770 and plants, plant cells, seed, plant parts, progeny plants, and commodity products comprising event GM_CSM63770; and methods related to detection, characterization, and selection of event GM_CSM63770.

### CROSS REFERENCE TO RELATED APPLICATION

This application claims the benefit of U.S. provisional application No. 63/355,947 filed Jun. 27, 2022, which is herein incorporated by reference in its entirety.

### INCORPORATION OF SEQUENCE LISTING

The sequence listing contained in the file named MONS567US_ST26 is 341,230 bytes (measured in Microsoft Windows®), was created on Jun. 16, 2023, is filed herewith by electronic submission, and is incorporated by reference.

### FIELD OF THE INVENTION

The invention relates to recombinant DNA molecules present in and/or isolated from soybean event GM_CSM63770. The invention also relates to transgenic soybean plants, plant parts, and seed, cells, and agricultural products containing soybean event GM_CSM63770, as well as methods of using the same and detecting the presence of soybean event GM_CSM63770. Transgenic soybean plants, plant parts, seed and cells containing soybean event GM_CSM63770 DNA exhibit resistance to insect infestations in the family Lepidoptera.

### BACKGROUND OF THE INVENTION

Soybean (Glycine max) is an important crop and is a primary food source in many areas of the world. The methods of biotechnology have been applied to soybean for improvement of the agronomic traits and quality of the product. One such agronomic trait is insect resistance, which is accomplished through the expression of heterologous insect toxins, also known as transgenes, inserted into the genome of the soybean plant.

There are a number of different transgenic events in soybean that have been described in the art that provide various types of insect resistance, particularly to Lepidopteran species, and these include MON87701, MON87751, and DAS81419 (Lepidopteran resistant and herbicide tolerant). These transgenic events have been in use commercially in a variety of geographies across the globe for an extended period of time, and resistance to the expressed toxins in these events by targeted insect pests has been observed in many geographic regions where these have been deployed.

Thus, there is a continuing need in the art to provide novel transgenic events in soybean that exhibit resistance to insect infestation, and preferably the novel transgenic events confer resistance to the target insects, including those races that have evolved resistance to the existing commercially deployed traits, using modes of action that are not overlapping with or similar to the modes of action previously deployed in earlier commercial embodiments. Described herein is an example of such a novel transgenic event that confers resistance to Lepidopteran infestations, including resistance to Lepidopterans that have evolved resistance to commercial embodiments that have been previously deployed.

### SUMMARY OF THE INVENTION

In one embodiment, the invention provides a novel transgenic soybean event—GM_CSM63770—that provides insecticidal control over Lepidopteran pests of soybean. In a further embodiment, the invention also provides transgenic plants, plant cells, seed, plant parts, and commodity products comprising soybean event GM_CSM63770 DNA. The event contains novel DNA that is specific and unique to this GM_CSM63770 event and comprises the inserted transgenic DNA segment and the novel DNA segments that are adjacent to the inserted DNA segment. These adjacent segments are described herein as the junction sequences which correspond to the DNA sequences extending along the chromosomal DNA adjacent to the chromosomal breakpoint at which the inserted DNA has been introduced. In another embodiment, the invention provides polynucleotides specific and unique to, and capable of use for identifying and detecting the presence of soybean event GM_CSM63770 DNA in a biological sample containing soybean tissue, as well as plant, plant cells, seed, plant parts, progeny plants, and commodity products comprising soybean event GM_CSM63770 DNA. In yet another embodiment, methods related to selecting plant cells, plants and seed comprising the soybean event GM_CSM63770 DNA, and detection of the presence (or absence) of soybean event GM_CSM63770 DNA in a sample are provided, such methods providing for the investigator to confirm that the event DNA is, or is not, present in a particular sample subjected to the method or to methods reliant upon nucleotide sequences that are the subject of this disclosure.

Thus, in one aspect the invention provides a recombinant DNA molecule comprising a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, and a complete complement thereof.

In one embodiment, the recombinant DNA molecule is derived from soybean event GM_CSM63770 in a sample of seed containing the event and which contains the corresponding unique and specific DNA segments corresponding to soybean event GM_CSM63770 seed which has been deposited as ATCC Accession No. PTA-126048.

Another aspect of the invention provides a DNA molecule comprising a polynucleotide segment of sufficient length to function as a DNA probe that hybridizes specifically under stringent hybridization conditions with soybean event GM_CSM63770 DNA in a sample, wherein detecting hybridization of the DNA probe to the DNA in the sample under the stringent hybridization conditions is diagnostic for, or characteristic of, confirming the presence of soybean event GM_CSM63770 DNA in that sample. In certain embodiments, the sample comprises a soybean plant, soybean plant cell, soybean seed, soybean pollen, soybean plant part, soybean progeny plant, process soybean seed, animal feed comprising soybean, soybean oil, soybean meal, soybean flour, soybean flakes, soybean bran, soybean biomass, and fuel products produced using soybean and soybean parts, provided that such soybean and soybean products contain detectable amounts of soybean event GM_CSM63770 DNA or detectable amounts of the novel toxin proteins produced by soybean plants, cells and the like, to contain the soybean event GM_CSM63770.

Yet another aspect of the invention provides a first DNA molecule and a second DNA molecule different from the first DNA molecule, i.e. a pair of DNA molecules that function as DNA primers when used together in an amplification reaction containing the appropriate reagents necessary for conducting a DNA amplification procedure with a sample containing soybean event GM_CSM63770 template DNA to produce an amplicon diagnostic for, or characteristic of, the presence of said soybean event GM_CSM63770 DNA in said sample. The amplicon produced will contain at least the nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, and the complete complement thereof.

Another embodiment of the invention is a method of detecting the presence of a DNA segment diagnostic for confirming the presence or absence of soybean event GM_CSM63770 in a sample. In certain embodiments, the method is conducted by contacting the sample with a probe DNA molecule that hybridizes specifically to DNA uniquely associated with soybean event GM_CSM63770, then subjecting the sample and the probe DNA molecule to stringent hybridization conditions to allow the probe to bind to the appropriate complement segment of soybean event GM_CSM63770 specific DNA. Detecting hybridization of the probe DNA molecule to the DNA in the sample is conclusive, diagnostic, determinative, that the DNA in the sample contains the soybean event GM_CSM63770 DNA.

Yet another embodiment of the invention is a method of detecting the presence of a DNA segment diagnostic for, or characteristic of, soybean event GM_CSM63770 DNA in a sample containing soybean DNA. In one embodiment, the method comprises the steps of contacting a biological sample with a pair of DNA molecules that function as thermal amplification primers specific for amplification of a segment of the soybean event GM_CSM63770 DNA; performing an amplification reaction sufficient to produce the DNA amplicon; and detecting the presence of the DNA amplicon in the reaction. Detection of the DNA amplicon is diagnostic for, or characteristic of, the presence of a detectable amount of the soybean event GM_CSM63770 DNA in the sample, and the amplicon will contain all or a portion of the nucleotide sequence targeted for amplification that lies between the primer hybridization positions, such portion of the nucleotide sequence targeted for amplification being selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, and the complete complement thereof.

Another embodiment of the invention is a method of detecting the presence of protein diagnostic for or characteristic of soybean event GM_CSM63770 in a sample, said method comprising: (a) contacting said sample with a first and second monoclonal antibody, wherein the first monoclonal antibody binds specifically to Cry1A.2 and the second monoclonal antibody binds specifically to Cry1B.2 in an immunoassay; (2) incubating the immunoassay for a sufficient amount of time to allow for binding of the monoclonal antibodies; and (3) detecting the presence of the Cry1A.2 and Cry1B.2 proteins in said immunoassay, wherein said detection is diagnostic for, or characteristic of, the presence of said soybean event GM_CSM63770 DNA in said sample. The assay can be selected from the group consisting of an Enzyme-linked Immunosorbent Assay (ELISA), a Radioimmunoassay, and a Lateral flow immunochromatographic assay.

Another embodiment of the invention is a soybean plant, soybean plant part, soybean cell, or part thereof comprising a recombinant polynucleotide molecule comprising the nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, and the complete complement thereof. These SEQ ID NOs are each the specific and selective DNA that defines the soybean event GM_CSM63770. This soybean plant, soybean plant part, soybean cell, or part thereof is insecticidal when provided in the diet of a Lepidopteran insect pest. Lepidopteran insect target pests intended to be controlled include Soybean podworm (Helicoverpa zea), Soybean looper (Chrysodeixis includens), Velvet bean caterpillar (Anticarsia gemmatalis), Southern armyworm (Spodoptera eridania), Black armyworm (Spodoptera cosmioides), South American podworm (Helicoverpa gelotopoeon), Sunflower looper (Rachiplusia nu), Bean shoot moth (Crocidosema aporema), Green cloverworm (Hypena scabra) and Lesser cornstalk borer (Elasmopalpus lignosellus). In addition, the soybean plant can be further defined as progeny of any generation of a soybean plant comprising the soybean event GM_CSM63770, provided that the progeny also contains the specific and selective DNA that defines the soybean event GM_CSM63770.

Yet another embodiment of the invention is a method for protecting a soybean plant from insect infestation, wherein said method comprises providing in the diet of a Lepidopteran insect pest an insecticidally effective amount of cells or tissue of the soybean plant comprising soybean event GM_CSM63770. Contemplated Lepidopteran insect pests include Soybean podworm (Helicoverpa zea), Soybean looper (Chrysodeixis includens), Velvet bean caterpillar (Anticarsia gemmatalis), Southern armyworm (Spodoptera eridania), Black armyworm (Spodoptera cosmioides), South American podworm (Helicoverpa gelotopoeon), Sunflower looper (Rachiplusia nu), Bean shoot moth (Crocidosema aporema), Green cloverworm (Hypena scabra) and Lesser cornstalk borer (Elasmopalpus lignosellus).

Another embodiment of the invention is a method of producing an insect resistant soybean plant comprising: a) manually crossing by conventional breeding, two different soybean plants to produce progeny, wherein at least one of the two different soybean plants contains the soybean event GM_CSM63770 DNA; b) confirming in the seed arising from the breeding activity, and in the progeny plants and tissue grown from such seed, the presence of a DNA segment diagnostic for, or characteristic of, soybean event GM_CSM63770; and c) selecting the progeny comprising the soybean event GM_CSM63770 DNA. Such seed and progeny are Lepidopteran resistant soybean plants.

A further embodiment of the invention is a soybean seed, nonliving plant material, or a microorganism comprising a detectable amount of the nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, or complete complements thereof.

Yet another embodiment is a commodity soybean product comprising a detectable amount of a DNA molecule unique to soybean event GM_CSM63770, wherein the molecule comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Contemplated commodity soybean products include, but are not limited to, whole or processed soybean seed, animal feed comprising soybean, soybean oil, soybean meal, soybean flour, soybean flakes, soybean bran, soybean biomass, and fuel products produced using soybean and soybean parts.

Another embodiment of the invention is a soybean plant, soybean plant part, or soybean seed thereof comprising DNA functional as a template when tested in DNA amplification method producing an amplicon diagnostic for, or characteristic of, the presence of soybean event GM_CSM63770 DNA.

Yet another embodiment of the invention is a method of determining the zygosity of a soybean plant or soybean seed comprising DNA descriptive of the soybean event GM_CSM63770. The zygosity is determined in a series of consecutive steps. In the first step, a sample comprising soybean DNA is contacted with a first primer pair that is capable of producing an amplicon diagnostic for, or characteristic of, DNA that is descriptive of and present exclusively in soybean event GM_CSM63770. Then the sample comprising the soybean DNA is contacted with a second primer pair that is designed to produce an amplicon of an internal standard known to be single-copy and homozygous in the soybean plant. The method additionally includes contacting the DNA sample with a probe set which contains at least a first probe that specifically hybridizes the allele of soybean event GM_CSM63770, and a second probe that specifically hybridizes to the internal standard genomic DNA known to be single-copy and homozygous in the soybean plant. The method also includes a DNA amplification reaction is performed using real-time PCR and determining the cycle thresholds (Ct values) of the amplicon corresponding the allele of soybean event GM_CSM63770 and the single-copy, homozygous internal standard. After the amplification, the difference (ΔCt) between the Ct value of the single-copy, homozygous internal standard amplicon, and the Ct value of the allele for soybean event GM_CSM63770 amplicon may be calculated. In one embodiment, zygosity is determined wherein a ΔCt of about zero (0) indicates homozygosity of the inserted T-DNA of soybean event GM_CSM63770 and a ΔCt of about one (1) indicates heterozygosity of the inserted T-DNA of soybean event GM_CSM63770. In certain embodiments of this method, the primer pairs are selected from the group consisting of SEQ ID NO:14 combined with SEQ ID NO:15, and SEQ ID NO:17 combined with SEQ ID NO:18; and wherein the probes are SEQ ID NO:16 and SEQ ID NO:19. In yet another embodiment of this invention the ΔCt of about one (1) indicating heterozygosity of the inserted T-DNA of corn event MON95275 is in the range of 0.75 to 1.25. In certain embodiments, a ΔCt of about zero (0) may be about 0, 0.05, 0.1, 0.15, 0.2, or 0.25, in other embodiments, a ΔCt of about one (1) may be about 0.75, 0.8, 0.85, 0.9, 0.95, 1.0, 1.05, 1.1, 1.15, 1.2, or 1.25. In a further embodiment, a ΔCt of about one (1) may be in the range of 0.75 to 1.25, 0.8 to 1.25, 0.85 to 1.25, 0.9 to 1.25, 0.95 to 1.25, 1.0 to 1.25, 1.05 to 1.25, 1.1 to 1.25, 1.15 to 1.25, 1.2 to 1.25, 0.75 to 1.2, 0.8 to 1.2, 0.85 to 1.2, 0.9 to 1.2, 0.95 to 1.2, 1.0 to 1.2, 1.05 to 1.2, 1.1 to 1.2, 1.15 to 1.2, 0.75 to 1.15, 0.8 to 1.15, 0.85 to 1.15, 0.9 to 1.15, 0.95 to 1.15, 1.0 to 1.15, 1.05 to 1.15, 1.1 to 1.15, 0.75 to 1.1, 0.8 to 1.1, 0.85 to 1.1, 0.9 to 1.1, 0.95 to 1.1, 1.0 to 1.1, 1.05 to 1.1, 0.75 to 1.05, 0.8 to 1.05, 0.85 to 1.05, 0.9 to 1.05, 0.95 to 1.05, 1.0 to 1.05, 0.75 to 1.0, 0.8 to 1.0, 0.85 to 1.0, 0.9 to 1.0, 0.95 to 1.0, 0.75 to 0.95, 0.8 to 0.95, 0.85 to 0.95, 0.9 to 0.95, 0.75 to 0.9, 0.75 to 0.85, 0.75 to 0.8, 0.8 to 0.9, 0.8 to 0.85, or 0.85 to 0.9.

A further embodiment of the invention is a method of determining the zygosity of a soybean plant, soybean seed, soybean pollen, ovum, or cell, in which each such biological component suspected of containing soybean event GM_CSM63770 DNA is subjected to the following method: a) contacting a sample containing DNA obtained from the suspect biological component with at least two different sets of primers, (i) in which a first primer pair consisting of a first primer and a second primer different from the first, when used in an amplification reaction together with soybean DNA, are capable of producing a first amplicon diagnostic for, or characteristic of, the presence of soybean event GM_CSM63770 DNA in the sample, and (ii) a second primer pair consisting of the first primer and a third primer different from the first primer and from the second primer, when used in an amplification reaction together with soybean DNA, are capable of producing a second amplicon diagnostic for, or characteristic of, native soybean genomic DNA which does not contain or include DNA specific for the soybean event GM_CSM63770; b) performing a nucleic acid amplification reaction with the sample and the two primer pairs; c) detecting in the nucleic acid amplification reaction the first amplicon diagnostic for, or characteristic of, soybean event GM_CSM63770, or the second amplicon diagnostic for, or characteristic of, native soybean genomic DNA not comprising soybean event GM_CSM63770, wherein the presence of only the first amplicon diagnostic of, or characteristic of, a homozygous soybean event GM_CSM63770 DNA in a sample, and the presence of both the first amplicon and the second amplicon is diagnostic of, or characteristic of, a soybean plant heterozygous for soybean event GM_CSM63770 allele; or b) contacting a sample comprising soybean DNA with a probe set which contains at least a first probe that specifically hybridizes to soybean event GM_CSM63770 DNA and at least a second probe that specifically hybridizes to soybean genomic DNA across the segment of chromosomal DNA that was disrupted by insertion of the heterologous DNA of soybean event GM_CSM63770 and which does not hybridize to soybean event GM_CSM63770 DNA; i) hybridizing the probe set with a sample under stringent hybridization conditions, wherein detecting hybridization of only the first probe under the hybridization conditions is diagnostic for, or characteristic of, a homozygous allele of soybean event GM_CSM63770 DNA in the sample, and wherein detecting hybridization of both the first probe and the second probe under the hybridization conditions is diagnostic for, or characteristic of, a heterozygous allele of soybean event GM_CSM63770 in said sample. In one embodiment of this method, the set of primer pairs comprises SEQ ID NO:14 combined with SEQ ID NO:15, and SEQ ID NO:20 combined with SEQ ID NO:15. In another embodiment of this method, the probe set comprises SEQ ID NO:16 and SEQ ID NO:21.

Yet another embodiment of the invention are soybean plants, plant cells, pollen, plant parts, and seed are provided which comprise a recombinant DNA construct integrated in chromosome 19. The recombinant DNA construct confers resistance to Lepidopteran insect pest species. The recombinant DNA construct is integrated in a position of said chromosome flanked by at least 50 contiguous nucleotides of SEQ ID NOs:11 or 36 and 50 contiguous nucleotides of SEQ ID NOs:12 or 37. The at least 50 contiguous nucleotides of SEQ ID NO:11 can comprise one or more nucleotide sequences selected from SEQ ID NOs:118-137. The at least 50 contiguous nucleotides of SEQ ID NO:36 can comprise one or more nucleotide sequences selected from SEQ ID NOs:38-117. The at least 50 contiguous nucleotides of SEQ ID NO:12 can comprise on or more nucleotide sequences selected from SEQ ID NOs:138-157. The at least 50 contiguous nucleotides of SEQ ID NO:37 can comprise one or more nucleotide sequences selected from SEQ ID NOs:158-237.

The forgoing and other aspects of the invention will become more apparent from the following detailed description.

### BRIEF DESCRIPTION OF THE DRAWINGS

FIG. 1 is a graphical depiction of the orientation and alignment of the DNA elements/segments that are present within the nucleotide sequence of SEQ ID NO:10 represented in the drawing as [10], which is the sequence of the inserted transgenic DNA and adjacent 5′ and 3′ sequences of the soybean genome present within the soybean event GM_CSM63770. SEQ ID NO:1 is represented by [1] and is a fifty (50) nucleotide segment spanning twenty five (25) nucleotides of the inserted DNA and twenty five (25) nucleotides of the arbitrarily assigned 5′ soybean chromosomal DNA segment adjacent to the inserted DNA. SEQ ID NO:2 is represented by [2] and is a fifty (50) nucleotide segment spanning twenty five (25) nucleotides of the inserted DNA and twenty five (25) nucleotides of the arbitrarily assigned 3′ soybean chromosomal DNA segment adjacent to the inserted DNA. SEQ ID NO:1 is embedded within SEQ ID NO:3 represented by [3], which is a one hundred (100) nucleotide segment spanning fifty (50) nucleotides of the inserted DNA and fifty (50) nucleotides of the arbitrarily assigned 5′ soybean chromosomal DNA segment adjacent to the inserted DNA. SEQ ID NO:2 is embedded within SEQ ID NO:4 represented by [4], which is a one hundred (100) nucleotide segment spanning fifty (50) nucleotides of the inserted DNA and fifty (50) nucleotides of the arbitrarily assigned 3′ soybean chromosomal DNA segment adjacent to the inserted DNA. SEQ ID NO:5 is represented by [5], which is two hundred (200) nucleotides in length, and contains SEQ ID NO:3, and spans one hundred (100) nucleotides of the inserted DNA and one hundred (100) nucleotides of the arbitrarily assigned 5′ soybean chromosomal DNA segment adjacent to the inserted DNA. SEQ ID NO:6 is represented by [6], which is two hundred (200) nucleotides in length, contains SEQ ID NO:4, and spans one hundred (100) nucleotides of the inserted DNA and one hundred (100) nucleotides of the arbitrarily assigned 3′ soybean chromosomal DNA segment adjacent to the inserted DNA. [7] is representative of SEQ ID NO:7 corresponding to one thousand (1,000) consecutive nucleotides of the soybean genome DNA at the arbitrarily assigned 5′ end of the inserted DNA, and one hundred eighty-one (181) consecutive nucleotides of the adjacent inserted transgenic DNA. [8] is representative of SEQ ID NO:8 corresponding to one thousand (1,000) consecutive nucleotides of the adjacent soybean genome DNA at the arbitrarily assigned 3′ end of the inserted DNA and one hundred ninety two (192) consecutive nucleotides of the 3′ end of the inserted transgenic DNA. [9] is representative of SEQ ID NO:9 and is the full length nucleotide segment of the inserted transgenic DNA. The arrows, and the labels below each arrow, represent the orientation of the direction of transcription and translation, as applicable, from the applicable expression elements that are positioned within the two cassettes within the inserted DNA. RB and LB represent the positions of the right and left borders of the Agrobacterium double border mediated transformation vector, letter P represents the positions of the promoter elements in the respective constructs, letter L represents the positions of leader sequences (5′ untranslated regions, 5′UTR) in the respective constructs, letter I represents the position of the intron sequence in the respective construct, the letter T represents the positions of the transcription termination sequences (3′ untranslated regions, 3′UTR) in the respective constructs. Elements from a P to the immediately following T represented in the graphic depiction at [9] and at [10] each represent a construct. The construct at the left side of FIG. 1 encodes a pest toxic Cry1A.2, and the construct at the right side of FIG. 1 encodes a Cry1B.2. Cry1A.2 and Cry1B.2 are each a different unique toxins, exhibiting less than 45% amino acid sequence identity to each other, each are toxic to Lepidopteran pests, and each toxin exhibits a different mode of action relative to the other. The position within the transgenic inserted DNA of each of the genetic elements involved in expression of these respective toxin proteins are specified in Table 1. [11] represents SEQ ID NO:11 and illustrates the position of the soybean genome DNA flanking the 5′ end of the inserted DNA. [12] represents SEQ ID NO:12 and illustrates the position of the soybean genome DNA flanking the 3′ end of the inserted DNA. [14] represents SEQ ID NO:14 or primer SQ13805. [15] represents SEQ ID NO:15, or primer SQ51400). These are described for use together, as a primer pair that can be applied to a sample containing event GM_CSM63770 DNA that, when used in a thermal amplification reaction, produces an amplicon of one hundred thirty four (134) nucleotides containing the 3′ end insert/genome junction, the arrows showing the approximate hybridization position within [10] and the direction in which transcription proceeds during the amplification cycles. [16] represents SEQ ID NO:16 or probe PB4832, which binds (or hybridizes to) an amplicon produced using, for example, primer [14] and [15] together in an amplification reaction with soybean event GM_CSM63770 DNA as template, for detecting the presence of the soybean event GM_CSM63770 DNA in a sample. [20] represents SEQ ID NO:20 or primer GM_WT63770F. [20] is a primer that binds to (or hybridizes to) the soybean genome DNA that is adjacent 5′ to the inserted DNA, and when combined with [15] (SEQ ID NO:15, primer SQ51400) in a thermal amplification reaction together with conventional soybean DNA as template lacking or devoid of soybean event GM_CSM63770 DNA, produces an amplicon of one hundred thirty six (136) nucleotides containing undisrupted soybean genome DNA, and detection of that amplicon is representative of a sample which does not contain the soybean transgenic event GM_CSM63770 DNA at that chromosomal locus. Probe GM_WT63770PB (SEQ ID NO:21) is representative of a probe that would bind (or hybridize to) the amplicon produced using primers [20] and [15], for detecting an allele lacking, or devoid of, event GM_CSM63770 DNA. Probe GM_WT63770PB hybridizes to the 3′ terminal 9 nucleotides of the 5′ genomic flanking DNA, the 14 nucleotides of the wild-type allelic DNA that was deleted during insertion of the T-DNA in soybean event GM_CSM63770, and the 5′ 2 nucleotides of the 3′ genomic DNA flanking the DNA corresponding to the transgenic inserted DNA described herein as event GM_CSM63770.

### BRIEF DESCRIPTION OF THE SEQUENCES

SEQ ID NO:1 is a 50-nucleotide sequence representing the 5′ junction region of soybean genomic DNA and the integrated transgenic expression cassette (25 nucleotides soybean genome DNA at the 5′ end of SEQ ID NO:1, 25 nucleotides transgenic inserted DNA at the 3′ end of SEQ ID NO:1), and can be identified within SEQ ID NO:10 at nucleotide positions 976-1,025.

SEQ ID NO:2 is a 50-nucleotide sequence representing the 3′ junction region of the integrated transgenic expression cassette and the soybean genomic DNA (25 nucleotides transgenic inserted DNA at the 5′ end of SEQ ID NO:2, 25 nucleotides soybean genome DNA at 3′ end of SEQ ID NO:2), and can be identified within SEQ ID NO:10 at nucleotide positions 13,216-13,265.

SEQ ID NO:3 is a 100-nucleotide sequence representing the 5′ junction region of soybean genomic DNA and the integrated transgenic expression cassette (50 nucleotides soybean genome DNA at the 5′ end of SEQ ID NO:3, 50 nucleotides transgenic inserted DNA at the 3′ end of SEQ ID NO:3), and can be identified within SEQ ID NO:10 at nucleotide positions 951-1,050.

SEQ ID NO:4 is a 100-nucleotide sequence representing the 3′ junction region of the integrated transgenic expression cassette and the soybean genomic DNA (50 nucleotides transgenic inserted DNA at the 5′ end of SEQ ID NO:4, 50 nucleotides soybean genome DNA at 3′ end of SEQ ID NO:4), and can be identified within SEQ ID NO:10 at nucleotide positions 13,191-13,290.

SEQ ID NO:5 is a 200-nucleotide sequence representing the 5′ junction region of soybean genomic DNA and the integrated transgenic expression cassette (100 nucleotides soybean genome DNA at the 5′ end of SEQ ID NO:5, 100 nucleotides transgenic inserted DNA at the 3′ end of SEQ ID NO:5), and can be identified within SEQ ID NO:10 at nucleotide positions 901-1,100.

SEQ ID NO:6 is a 200-nucleotide sequence representing the 3′ junction region of the integrated transgenic expression cassette and the soybean genomic DNA (100 nucleotides transgenic inserted DNA at the 5′ end of SEQ ID NO:6, 100 nucleotides soybean genome DNA at 3′ end of SEQ ID NO:6), and can be identified within SEQ ID NO:10 at nucleotide positions 13,141-13,340.

SEQ ID NO:7 is a 1,181-nucleotide sequence representing the 5′ junction region of soybean genomic DNA and the integrated transgenic expression cassette (1,000 nucleotides soybean genome DNA at the 5′ end of SEQ ID NO:7, 181 nucleotides soybean genome DNA at 3′ end of SEQ ID NO:7) and can be identified within SEQ ID NO:10 at nucleotide positions 1-1,181.

SEQ ID NO:8 is a 1,192-nucleotide sequence representing the 3′ junction region of the integrated transgenic expression cassette and the soybean genomic DNA (192 nucleotides transgenic inserted DNA at the 5′ end of SEQ ID NO:6, 1000 nucleotides soybean genome DNA at 3′ end of SEQ ID NO:6), and can be identified within SEQ ID NO:10 at nucleotide positions 13,049-14,240.

SEQ ID NO:9 is a 12,240-nucleotide sequence corresponding to the transgenic inserted T-DNA of soybean event GM_CSM63770, and can be identified within SEQ ID NO:10 at nucleotide positions 1,001-13,240.

SEQ ID NO:10 is a 14,240-nucleotide sequence corresponding to the contig nucleotide sequence of the 5′ genomic flanking DNA nucleotide sequence, the inserted T-DNA nucleotide sequence in event GM_CSM63770, and the 3′ genomic flanking DNA nucleotide sequence; and includes SEQ ID NO:11 (nucleotides 1-1,1000), SEQ ID NO:9 (nucleotides 1,001-13,240), and SEQ ID NO:12 (nucleotides 13,241-14,240).

SEQ ID NO:11 is a 1,000-nucleotide sequence representing the 5′ flanking soybean genomic DNA up to the inserted T-DNA, and can be identified within SEQ ID NO:10 at nucleotide positions 1-1,000.

SEQ ID NO:12 is a 1,000-nucleotide sequence representing the 3′ flanking soybean genomic DNA after the inserted T-DNA, and can be identified within SEQ ID NO:10 at nucleotide positions 13,241-14,240.

SEQ ID NO:13 is a 12,629-nucleotide sequence representing the transgene cassette comprised within the binary plasmid transformation vector used to transform soybean to produce soybean event GM_CSM63770.

SEQ ID NO:14 is a 26-nucleotide sequence corresponding to a thermal amplification primer referred to as SQ13805 which can be used to identify soybean event GM_CSM63770 DNA in a sample, and is identical to the nucleotide sequence corresponding to positions 13,177-13,202 of SEQ ID NO:10.

SEQ ID NO:15 is a 31-nucleotide sequence corresponding to a thermal amplification primer referred to as SQ51400 which can be used to identify soybean event GM_CSM63770 DNA in a sample, and is identical to the reverse complement of the nucleotide sequence corresponding to positions 13,280-13,310 of SEQ ID NO:10.

SEQ ID NO:16 is a 16-nucleotide sequence corresponding to a probe referred to as PB4832 used to identify soybean event GM_CSM63770 DNA in a sample, and is identical to the nucleotide sequence corresponding to positions 13,204-13,219 of SEQ ID NO:10.

SEQ ID NO:17 is a 20-nucleotide sequence corresponding to a thermal amplification primer referred to as SQ549 used as an internal control for the event and zygosity assay for soybean event GM_CSM63770 and hybridizes to a region of the soybean genome.

SEQ ID NO:18 is a 20-nucleotide sequence corresponding to a thermal amplification primer referred to as SQ546 used as an internal control for the event and zygosity assay for soybean event GM_CSM63770 and hybridizes to a region of the soybean genome.

SEQ ID NO:19 is a 28-nucleotide sequence corresponding to a probe referred to as PB0004 used as an internal control for the event and zygosity assay for soybean event GM_CSM63770 and hybridizes to a region of the soybean genome.

SEQ ID NO:20 is a 25-nucleotide sequence corresponding to a thermal amplification primer referred to as GM_WT63770F used in the zygosity assay for soybean event GM_CSM63770 and hybridizes to the 5′ genomic flanking DNA, and is identical to the nucleotide sequence corresponding to positions 949-971 of SEQ ID NO:10.

SEQ ID NO:21 is a 25-nucleotide sequence corresponding to a probe referred to as GM_WT63770PB used in the zygosity assay for soybean event GM_CSM63770 and hybridizes to the last 9 nucleotides of the 5′ genomic flanking DNA, the 14 nucleotides of the wild-type allelic DNA that was deleted during insertion of the T-DNA in soybean event GM_CSM63770, and the first 2 nucleotides of the 3′ genomic flanking DNA.

SEQ ID NO:22 is a 27-nucleotide sequence corresponding to an originator guide RNA recognition site (OgRRS), OgRRS_5-1 comprised of a Cas12a protospacer adjacent motif (PAM) site operably linked to a guide-RNA hybridization site.

SEQ ID NO:23 is a 27-nucleotide sequence corresponding to an OgRRS, OgRRS_5-2.

SEQ ID NO:24 is a 27-nucleotide sequence corresponding to an OgRRS, OgRRS_5-3.

SEQ ID NO:25 is a 27-nucleotide sequence corresponding to an OgRRS, OgRRS_3-1.

SEQ ID NO:26 is a 27-nucleotide sequence corresponding to an OgRRS, OgRRS_In-1.

SEQ ID NO:27 is a 27 nucleotide sequence corresponding to an OgRRS, OgRRS_In-2.

SEQ ID NO:28 is a 51-nucleotide sequence corresponding to a guide-RNA (gRNA), gRNA_OgRRS_5-1.

SEQ ID NO:29 is a 51-nucleotide sequence corresponding to a gRNA, gRNA_OgRRS_5-2.

SEQ ID NO:30 is a 51-nucleotide sequence corresponding to a gRNA, gRNA_OgRRS_5-3.

SEQ ID NO:31 is a 51-nucleotide sequence corresponding to a gRNA, gRNA_OgRRS_3-1.

SEQ ID NO:32 is a 51-nucleotide sequence corresponding to a gRNA, gRNA_OgRRS_In-1.

SEQ ID NO:33 is a 51-nucleotide sequence corresponding to a gRNA, gRNA_OgRRS_In-2.

SEQ ID NO:34 is a sequence of a synthetic DNA coding sequence designed for expression in a plant cell encoding a nuclear targeted Cas12a CRISPR-associated protein.

SEQ ID NO:35 is an amino acid sequence of a nuclear targeted Cas12a CRISPR-associated protein.

SEQ ID NO:36 is a 5,000-nucleotide sequence representing soybean genomic DNA that flanks the transgenic insert at the 5′end of the insert. Nucleotides 4,001-5,000 of SEQ ID NO:36 are identical to nucleotides 1-1,000 of SEQ ID NO:11.

SEQ ID NO:37 is a 5,000-nucleotide sequence representing soybean genomic DNA that flanks the transgenic insert at the 3′ end of the insert. Nucleotides 1-1,000 of SEQ ID NO:99 are identical to nucleotides 1-1000 of SEQ NO: 12. The remaining nucleotides of SEQ ID NO:99 (nucleotides 1,001-5,000) are based on the genomic sequence of the Williams 82 soybean cultivar.

SEQ ID NOs:38-117 are additional 50-nucleotide sequence in the 5′ flank genomic sequence of event GM_CSM63770 based on the genomic sequence of the Williams 82 soybean cultivar.

SEQ ID NOs:118-137 are 50-nucleotide sequence in the 5′ flank genomic sequence of event GM_CSM63770.

SEQ ID NOs:138-157 are 50-nucleotide sequence in the 3′ flank genomic sequence of event GM_CSM63770.

SEQ ID NOs:158-237 are additional 50-nucleotide sequence in the 3′ flank genomic sequence of event GM_CSM63770 based on the genomic sequence of the Williams 82 soybean cultivar.

### DETAILED DESCRIPTION

The present invention provides a transgenic soybean event GM_CSM63770 that achieves insecticidal control over certain Lepidopteran larval pests of soybean by expression of insecticidal toxins Cry1A.2 and Cry1B.2, which are available in the tissues of the soybean plant containing this event and presented to the larval pests when they consume the plant tissues. Specifically, expression of the Cry1A.2 and Cry1B.2 insect inhibitory proteins in soybean event GM_CSM63770 provides resistance to the larval forms of Lepidopteran insect pests Soybean podworm (SPW, Helicoverpa zea), Soybean looper (Chrysodeixis includens), Velvet bean caterpillar (Anticarsia gemmatalis), Southern armyworm (Spodoptera eridania), Black armyworm (Spodoptera cosmioides), South American podworm (Helicoverpa gelotopoeon), Sunflower looper (Rachiplusia nu), Bean shoot moth (Crocidosema aporema), Green cloverworm (Hypena scabra) and Lesser cornstalk borer (Elasmopalpus lignosellus). Event GM_CSM63770 provides an unsolved need in the art for control of these insects in the soybean market, because Lepidopteran species have begun to develop resistance to the pest control proteins used in earlier versions of transgenic soybean plants expressing such Lepidopteran control proteins, and chemical insecticides have not provided adequate control of these insects and at times multiple applications of chemistries are required during the growing season, increasing the input of chemical pesticides in the environment, increasing the carbon footprint at each application, and adding significant cost to the production of the soybean crop.

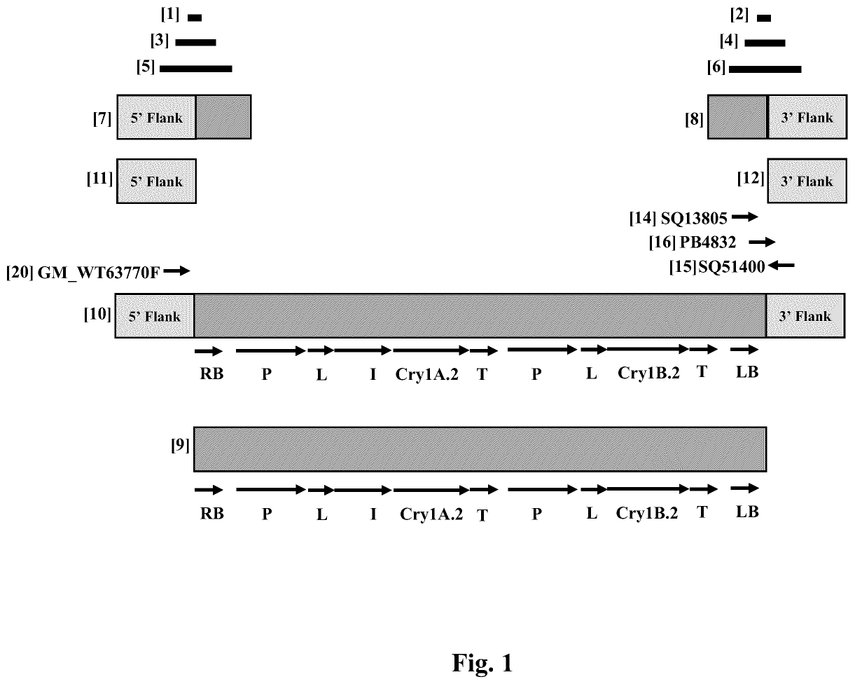

The protection against infestation by Lepidopteran species that is provided by event GM_CSM63770 arises in connection with the expression of a DNA segment encoding two different Lepidopteran specific insecticidal proteins that are operably and covalently linked within the inserted transgenic DNA that in part defines the soybean event GM_CSM63770. The two insecticidal proteins encoded by the transgenic inserted DNA in the soybean event GM_CSM63770 are (i) Cry1A.2 (U.S. Pat. No. 10,494,408, the amino acid sequence being referenced therein as SEQ ID NO:4, and the coding sequence as SEQ ID NO:3), and (ii) Cry1B.2 (U.S. Pat. No. 10,669,3017, the amino acid sequence being referenced therein as SEQ ID NO:10, and the coding sequence as SEQ ID NO:9). These two insecticidal proteins are described herein as being expressed from two different but linked expression cassettes within the inserted transgenic DNA construct as set forth herein in SEQ ID NO:9 and as illustrated in FIG. 1.

The DNA sequence encoding the Cry1A.2 protein in soybean event GM_CSM63770 is operably linked to an Arabidopsis thaliana Ubiquitin 10 promoter, leader, and intron. The DNA sequence encoding the Cry1B.2 protein in soybean event GM_CSM63770 is operably linked to a Cucumis melo chlorophyll a-b binding protein 13 gene promoter and leader (U.S. Pat. No. 10,443,066, referenced therein as SEQ ID NOs:4-6). Expression (transcription into the mRNA's coding for the toxin amino acid sequences, and translation of the mRNAs into the toxin proteins) of the toxin proteins Cry1A.2 and Cry1B.2 from their respective transgene cassettes is oriented in the same direction (head to tail/head to tail). FIG. 1 shows the relative positions of each element-promoter (P), 5′ UTR or leader (L), intron (I), toxin coding sequence or ORF (open reading frame), and 3′ UTR (T), the ORF for Cry1A.2, and ORF for Cry1B.2 being comprised within SEQ ID NO:9 and SEQ ID NO:10 respectively.

As described herein, numerous constructs which varied with respect to the use of expression elements, toxin coding sequences, and orientation of transcription and translation were evaluated. One hundred twenty-five (125) constructs, comprising one or more of twenty-one (21) different insect toxin coding sequences, were used to generate events for assay, leading to the selection of soybean event GM_CSM63770. The construct used to create soybean event GM_CSM63770, presented herein as SEQ ID NO:13, provided superior performance relative to other constructs when evaluated for the corresponding transgenic soybean plants' resistance to Lepidopteran insect pest infestation. In addition, soybean event GM_CSM63770 is free of the markers used for selection of the transformed plant cell as a result of method of transformation used ensuring the selectable marker and the intended traits to be integrated are able to be segregated from each other by breeding selection. Soybean event GM_CSM63770 was transformed with a two T-DNA plant transformation binary vector, wherein the selection cassettes were comprised on a separate T-DNA than the T-DNA which comprised the insect toxin transgene expression cassettes retained in soybean event GM_CSM63770. After self-pollination of the R₀ generation transformed soybean plants, R₁ progeny were selected for the presence of the T-DNA comprising the insect toxin expression cassettes and absence of the T-DNA used for selection of the initial transformation event.

Soybean event GM_CSM63770 was created through plant transformation techniques used to insert heterologous DNA (also known as transgenic DNA) randomly into a chromosome of the genome of a soybean cell to produce a genetically engineered soybean cell, also referred to as a “transgenic” or “recombinant” soybean cell. Using this technique, many individual cells are transformed, each resulting in a unique “transgenic event” or “event” due to the random insertion of the foreign DNA into the genome. A transgenic plant is then regenerated from each individual transgenic cell. This results in every cell of the transgenic plant containing the uniquely inserted transgenic event as a stable part of its genome. This transgenic plant can then be used to produce seed which are then planted and grown into progeny plants, each containing the unique transgenic event.

Soybean event GM_CSM63770 was produced by an Agrobacterium-mediated transformation process using the binary transformation plasmid construct GM_CSM63770 and dried excised soybean plant explants. This plasmid construct contains two regions, each bounded by Agrobacterium border segments (T-DNA segment). The first T-DNA segment contains two linked plant expression cassettes, one expression cassette encoding a selectable marker and one expression cassette encoding a scorable marker. The second T-DNA segment contains two linked plant expression cassettes with the regulatory genetic elements necessary for expression in soybean plant cells of two different insecticidal proteins, Cry1A.2 and Cry1B.2. The T-DNA segment containing the selection/scorable marker genes inserted randomly into the soybean genome and at a site separate from the site of integration of the T-DNA segment containing the Cry1A.2 and Cry1B.2 expression cassettes, thus allowing for segregation of the two T-DNA segments within the genome of the transformed plants during a process of selfing and/or backcrossing, e.g., screening R₁ and higher generation of transgenic plants. The transformed soybean cells were regenerated into intact soybean plants and individual plants were selected from the population of plants that showed integrity of the second T-DNA segment encoding the Cry1A.2 and Cry1B.2 proteins. In R₁ and subsequent generations, events were selected based on the integrity, stability and intactness of the T-DNA segment encoding the Cry1A.2 and Cry1B.2 proteins, on the absence (i.e., segregation) of (i) the T-DNA segment encoding the selectable/scorable marker cassettes, and (ii) any plasmid backbone sequence. Further selection was also made based upon the location of the T-DNA segment encoding the Cry1A.2 and Cry1B.2 proteins within the soybean genome and other characteristics such as efficacy and agronomics. The expression of the Cry1A.2 and Cry1B.2 insecticidal toxic proteins in the cells of the soybean event GM_CSM63770 confers resistance to Lepidopteran insect pests when the soybean cells of event GM_CSM63770 are provided in the diet of the insects.

The T-DNA segment encoding the Cry1A.2 and Cry1B.2 proteins in plasmid construct pM63770 is presented as SEQ ID NO:13. During integration, two hundred seventeen (217) and one hundred sixty-nine (169) contiguous base pairs (bp) were deleted from the right and left borders, respectively. In addition, fourteen (14) contiguous base pairs (bp) of the wild-type genomic DNA at the point of insertion of the transgenic DNA was deleted during the integration process.

As specifically described herein, soybean event GM_CSM63770 was produced by a complex research and development process in which: (1) over one hundred plasmid vector constructs—which varied with respect to the coding sequences for the insecticidal proteins, the coding sequences for the transcriptional regulatory elements, and number and orientation of the cassettes within the constructs—were developed and transformed into soybean cells to create thousands of events that were tested and analyzed, resulting in the selection of the construct used to generate event GM_CSM63770; (2) thousands of soybean cells were transformed with the construct used to generate event GM_CSM63770, creating a population of transgenic plants in which each plant contained a unique transgenic event that was regenerated and tested; (3) the final event GM_CSM63770 was selected after a rigorous multi-year event selection process involving the testing and analysis of molecular characteristics, efficacy, protein expression, and agronomic properties in a variety of soybean genetic backgrounds. Soybean event GM_CSM63770 was thus produced and selected as a uniquely superior event useful for broad-scale agronomic purposes.

The transgenic DNA inserted into the genome of soybean event GM_CSM63770 was characterized by detailed molecular analysis. This analysis included: the insert number (number of integration sites within the soybean genome), the genomic insert location (the specific site in the soybean genome where the insertion occurred), the copy number (the number of copies of the T-DNA within one locus), and the integrity (absence of any rearrangements) of the transgenic inserted DNA. The detailed molecular analysis demonstrated that the integrated T-DNA containing the Cry1A.2 and Cry1B.2 expression cassettes remained intact after integration and the T-DNA comprising the selectable/scoreable markers was absent. As used herein, an “expression cassette” or “cassette” is a recombinant DNA molecule comprising a combination of distinct elements that are to be expressed by a transformed cell. Table 1 provides a list of the elements contained in SEQ ID NO:10, the DNA sequence that corresponds to soybean event GM_CSM63770.

TABLE 1
Description of soybean event GM_CSM63770.

| | Position in | |
|---|---|---|
| Element | SEQ ID NO: 10 | Description |
| 5′ Flanking | 1-1,000 | DNA sequence flanking the 5′ end of |
| DNA | | the transgenic insert. |
| Right Border | 1,001-1,068 | DNA region from Agrobacterium |
| Region | | tumefaciens containing the right border |
| | | sequence. |
| P-At.Ubq10- | 1,182-2,010 | The promoter from a polyubiquitin |
| 1:1:1 | | gene (UBQ10) from Arabidopsis |
| | | thaliana (thale cress). |
| L-At.Ubq10- | 2,011-2,077 | The 5′ untranslated region from a |
| 1:1:1 | | polyubiquitin gene (UBQ10) from |
| | | Arabidopsis thaliana (thale cress). |
| I-At.Ubq10:3 | 2,078-2,383 | The intron for a polyubiquitin gene |
| | | (UBQ10) from Arabidopsis thaliana |
| | | (thale cress). |
| Cry1A.2 | 2,393-5,962 | Coding sequence of a chimeric insect |
| | | toxin comprised of domain 1 of |
| | | Cry1Ah, domain 2 of Cry1Ac, domain |
| | | 3 of Cry1Ca, and the protoxin domain |
| | | of Cry1Ac. |
| T-Mt.Zfp- | 5,971-6,570 | The 3′ untranslated region for a |
| 1:2:1 | | putative CCCH-type zinc finger |
| | | protein from Medicago truncatula |
| | | (barrel medic). |
| P-CUCme. | 6,673-8,571 | The promoter for a LHCII type III |
| CipLhcb:1 | | chlorophyll a/b binding protein from |
| | | Cucumis melo (melon). |
| L-CUCme. | 8,572-8,673 | The 5′ untranslated region for a LHCII |
| CipLhcb:1 | | type III chlorophyll a/b binding protein |
| | | from Cucumis melo (melon). |
| Cry1B.2 | 8,677-12,240 | Coding sequence of a chimeric insect |
| | | toxin comprised of domains 1 and 2 of |
| | | Cry1Be, domain 3 of Cry1Ka, and a |
| | | protoxin domain of Cry1Ab. |
| T-Mt.Lox- | 12,241-12,740 | The 3′ untranslated region of a |
| 1-1:2:1 | | lipoxygenase gene from Medicago |
| | | truncatula (barrel medic). |
| Left Border | 12,971-13,240 | DNA region from Agrobacterium |
| Region | | tumefaciens containing the left border |
| | | sequence. |
| 3′ Flanking | 13,241-14,240 | DNA sequence flanking the 3′ end of |
| DNA | | the transgenic insert. |

Soybean event GM_CSM63770 is characterized as an insertion of the intended transgenic DNA into a single locus in the soybean genome, resulting in two new loci or junction sequences (e.g., sequences set forth in SEQ ID NO:1, SEQ ID NO:2, SEQ ID NO:3, SEQ ID NO:4, SEQ ID NO:5, SEQ ID NO:6, SEQ ID NO:7, and SEQ ID NO:8) between the inserted DNA and the soybean genome DNA that are not known to appear naturally in the soybean genome or other transgenic soybean events—they are unique to soybean DNA containing the event GM_CSM63770. These junction sequences are useful in detecting the presence of the event in soybean cells, soybean tissue, soybean seed, soybean pollen and ova, and soybean plants or soybean plant products, such as soybean commodity products. DNA molecular probes and primer pairs are described herein that have been developed for use in identifying the presence of these various junction segments in biological samples containing or suspected of containing soybean cells, soybean seed, soybean plant parts, soybean pollen or ova, or soybean plant tissue that contain the event GM_CSM63770.

A sample is intended to refer to a composition that is either substantially pure soybean DNA or protein, or a composition that contains soybean DNA or protein. With respect to a sample containing DNA, the sample is a biological sample, i.e., it contains biological materials, including but not limited to DNA obtained or derived from, either directly or indirectly, from the genome of soybean event GM_CSM63770. “Directly” refers to the ability of the skilled artisan to directly obtain DNA from the soybean genome by fracturing soybean cells (or by obtaining samples of soybean that contain fractured soybean cells) and exposing the genomic DNA for the purposes of detection. “Indirectly” refers to the ability of the skilled artisan to obtain the target or specific reference DNA, i.e., a novel and unique junction segment described herein as being diagnostic for, or characteristic of, the presence of the soybean event GM_CSM63770 in a particular sample, by means other than by direct via fracturing of soybean cells or obtaining a sample of soybean that contains fractured soybean cells. Such indirect means include, but are not limited to, amplification of a DNA segment that contains the DNA sequence targeted by a particular probe designed to bind with specificity to the target sequence, or amplification of a DNA segment that can be measured and characterized, i.e., measured by separation from other segments of DNA through some efficient matrix such as an agarose or acrylamide gel or the like, or characterized by direct sequence analysis of the amplicons, or cloning of the amplicon into a vector and direct sequencing of the inserted amplicon present within such vector.

A sample of pure soybean protein or a composition that contains soybean protein is a biological sample, i.e., it contains biological materials, including but not limited to protein obtained or derived from the tissues or cells of soybean event GM_CSM63770. The sample is contacted with monoclonal antibodies that bind specifically to Cry1A.2 and Cry1B.2 in an immunoassay. Detection of the bound proteins is diagnostic of, or characteristic of soybean event GM_CSM63770. The immunoassay method can be, but not limited to, an ELISA (Enzyme-Linked Immunosorbent Assay), a Radioimmunoassay, or a Lateral flow immunochromatographic assay. ELISA assays are typically performed in the laboratory using tissue or cell samples obtained from whole plants, or plant parts thereof. Sandwich ELISA assays are frequently used to detect and quantify protein from transgenic crops. To detect the presence of soybean event GM_CSM63770 in a sample using a sandwich ELISA assay, monoclonal antibodies are used, a first monoclonal antibody which binds specifically to Cry1A.2 and a second antibody which binds specifically to Cry1B.2. A known amount of monoclonal antibody is bound to a fixed surface. Nonspecific sites on the solid surface are blocked by bovine serum albumin, casein, or any other such neutral solution. The sample containing protein derived from soybean event GM_CSM63770 is applied to the plate and is captured by the antibody. The unbound antigens are washed away by washing solution. A secondary antibody is added which is also conjugated to an enzyme. The unbound antibodies are washed away. A substrate is added, and the enzyme reacts with the substrate and produces a product, typically a pigment or photons, which is proportional to the amount of antigen present. Separate ELISA assays are performed for each of the two toxin protein in a sample derived from soybean event GM_CSM63770. A positive ELISA reaction is diagnostic for, or characteristic of, soybean event GM_CSM63770.

Lateral flow immunochromatographic assays, also known as, immunochromatographic strips (ICS) or dipstick tests, can be used in the field where laboratory facilities are not available for more complex immunoassays. The immunochromatographic test strip is composed of a sample pad, conjugate pad, nitrocellulose membrane, absorbent pad, and a backing card. A first monoclonal antibody which binds specifically to Cry1A.2, a second monoclonal antibody which binds specifically to Cry1B.2 and IgG antibody which binds to antibodies derived from the organism host cells in which the first and second monoclonal antibodies were derived are transferred onto the nitrocellulose membrane to form test line 1 (Cry1A.2), test line 2 (Cry1B.2), and the control line (anti-host IgG antibody). The conjugate pad is coated with the first and second monoclonal antibodies labeled with colloidal gold nanoparticles. The blotted membrane, conjugate pad, sample pad, and absorbent pad are assembled sequentially on the plastic backing board. The Cry1A.2 and Cry1B.2 proteins present in a sample derived from soybean event GM_CSM63770 will combine with their respective gold-labeled monoclonal antibodies and then bind to capture antibodies coated on the test line 1 and test line 2. Visual detection of test line 1 and test line 2 is diagnostic for, or characteristic of, soybean event GM_CSM63770.

Detailed molecular analysis also demonstrated that event GM_CSM63770 contains a single T-DNA insertion with one copy of each of the Cry1A.2 and Cry1B.2 expression cassettes. No additional elements from the transformation construct other than portions of the Agrobacterium tumefaciens left and right border regions used for transgenic DNA transfer from the plant transformation plasmid to the soybean genome were identified in event GM_CSM63770. Further, thermal amplification producing specific amplicons diagnostic for, or characteristic of, the presence of event GM_CSM63770 in a sample and DNA sequence analyses were performed to determine the arbitrarily assigned 5′ and 3′ insert-to-plant genome junctions, confirm the organization of the elements within the insert, and determine the complete DNA sequence of the inserted transgenic DNA (SEQ ID NO:9). SEQ ID NO:11 is a sequence representing the one thousand (1,000) base-pair (bp) adjacent to the arbitrarily assigned 5′ end of the inserted DNA in soybean variety A3555 genomic DNA sequence flanking the inserted T-DNA sequence. SEQ ID NO:12 is a sequence representing the one thousand (1,000) bp adjacent to the arbitrarily assigned 3′ end of the inserted DNA in soybean variety A3555 genomic DNA sequence flanking the inserted T-DNA sequence. SEQ ID NO:7 is SEQ ID NO:11 plus one hundred eighty-one (181) bp of the 5′ end of the inserted T-DNA sequence added to the 3′ end of SEQ ID NO:11. SEQ ID NO:8 is SEQ ID NO:12 plus one hundred ninety-two (192) bp of the 3′ end of the inserted T-DNA sequence added to the 5′ end of SEQ ID NO:12. SEQ ID NO:10 corresponds to the DNA sequence that defines the soybean event GM_CSM63770 and contains a contiguous sequence (contig) comprising the 5′ A3555 flanking sequence, the transgene insert of GM_CSM63770, and the 3′ A3555 flanking sequence, and thus contains both of the insert-to-plant genome junction sequences specified as SEQ ID NO:1, 3, 5 and 7 (5′ end), and as SEQ ID NO:2, 4, 6, and 8 (3′ end).

Unless otherwise noted herein, terms are to be understood according to conventional usage by those of ordinary skill in the relevant art. Definitions of common terms in molecular biology may be found in Rieger et al., Glossary of Genetics: Classical and Molecular, 5^{th} edition, Springer-Verlag: New York, 1991; and Lewin, Genes V, Oxford University Press: New York, 1994, along with other sources known to those of ordinary skill in the art. As used herein, the term “soybean” means species belong to the genus Glycine, preferably Glycine max and includes all plant varieties that can be bred with soybean plants containing event GM_CSM63770, including wild soybean species as well as those plants belonging to the genus Glycine that permit breeding between species.

Event GM_CSM63770 was transformed with a DNA construct that contains expression cassettes expressing toxic amounts of the insecticidal proteins Cry1A.2 and Cry1B.2. What is meant by toxic amount is an efficacious amount, an insecticidal amount, an insecticidally-effective amount, a target insect suppressive amount, an efficacious pesticidal amount, an amount in the diet of insects in the order of Lepidoptera that is insecticidal, and other similar terms to be understood according to conventional usage by those of ordinary skill in the relevant art. Soybean plants transformed according to the methods and with the DNA construct disclosed herein are resistant to Lepidopteran insect pests.

A transgenic “plant” is produced by transformation of a plant cell with heterologous DNA, i.e., a polynucleic acid construct that includes a number of efficacious features of interest, regeneration of a plant resulting from the insertion of the transgene into the genome of the plant cell, and selection of a particular plant characterized by insertion into a particular genome location and the number of efficacious features of the regenerated transgenic plant. The term “event” refers to DNA from the original transformant comprising the inserted DNA and flanking genomic sequences immediately adjacent to the inserted DNA. Such DNA is unique and would be expected to be transferred to a progeny that receives the inserted DNA, including the transgene of interest, as the result of a sexual cross of parental line that includes the inserted DNA (e.g., the original transformant and progeny resulting from selfing) and a parental line that does not contain the inserted DNA. The present invention also provides the original transformant plant and progeny of the transformant that include the heterologous DNA. Such progeny may be produced by a sexual outcross between plants comprising the event and another plant wherein the progeny includes the heterologous DNA. Even after repeated back-crossing to a recurrent parent, the event is present in the progeny of the cross at the same chromosomal location. The transgenic event DNA, and the genome into which the transgenic event DNA is detectable within the plant cell, the plant, the seed, and other parts of the plant, are not pre-existent in nature.

As used herein, the term “recombinant” refers to a non-natural DNA, protein, or organism that would not normally be found in nature and was created by human intervention. A “recombinant DNA molecule” is a DNA molecule comprising a combination of DNA molecules that would not naturally occur together and is the result of human intervention. For example, a DNA molecule that is comprised of a combination of at least two DNA molecules heterologous to each other, such as a DNA molecule that comprises a transgene and the plant genomic DNA adjacent to the transgene, is a recombinant DNA molecule.

The terms “DNA” and “DNA molecule” referred to herein refer to a deoxyribonucleic acid (DNA) molecule. A DNA molecule may be of genomic or synthetic origin, and is by convention from the 5′ (upstream) end to the 3′ (downstream) end. As used herein, the term “DNA sequence” refers to the nucleotide sequence of the DNA molecule. By convention, the DNA sequences of the invention and fragments thereof are disclosed with reference to only one strand of the two-strand complementary DNA sequence strands. By implication and intent, the complementary sequences of the sequences provided here (the sequences of the complementary strand), also referred to in the art as the reverse complementary sequences, are within the scope of the invention and are expressly intended to be within the scope of the subject matter claimed.

As used herein, the term “fragment” refers to a smaller piece of the whole. For example, fragments of SEQ ID NO:10 would include sequences that are at least about 12 consecutive nucleotides, at least about 13 consecutive nucleotides, at least about 14 consecutive nucleotides, at least about 15 consecutive nucleotides, at least about 16 consecutive nucleotides, at least about 17 consecutive nucleotides, at least about 18 consecutive nucleotides, at least about 19 consecutive nucleotides, at least about 20 consecutive nucleotides, at least about 25 consecutive nucleotides, at least about 30 consecutive nucleotides, at least about 35 consecutive nucleotides, at least about 40 consecutive nucleotides, at least about 45 consecutive nucleotides, at least about 50 consecutive nucleotides, at least about 60 consecutive nucleotides, at least about 70 consecutive nucleotides, at least about 80 consecutive nucleotides, at least about 90 consecutive nucleotides, or at least about 100 consecutive nucleotides of the complete sequence of SEQ ID NO:10.

Similarly, a fragment of the 5′ flank (SEQ ID NO:11 or SEQ ID NO:36) or 3′ flank (SEQ ID NO:12 or SEQ ID NO:37) of soybean event GM_CSM63770 can comprise at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 400, or at least about 500 consecutive nucleotides of SEQ ID NO:11 or SEQ ID NO:36; or SEQ ID NO:12 or SEQ ID NO:37. In addition, the present disclosure encompasses nucleotide sequences that are at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO:11 or 12, or SEQ ID NO:36 or 37, or any fragment of either thereof.

Reference in this disclosure to an “isolated DNA molecule” or an equivalent term or phrase is intended to mean that the DNA molecule is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding an insecticidal protein or any naturally occurring insecticidal variant of that protein would be an isolated nucleotide sequence so long as the nucleotide sequence was not within the DNA of the bacterium from which the sequence encoding the protein is naturally found. A synthetic nucleotide sequence encoding the amino acid sequence of the naturally occurring insecticidal protein would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium. In any circumstance, the isolated DNA molecule is a chemical molecule, regardless of whether it is referred to as a nucleic acid, a nucleic acid sequence, a polynucleotide sequence, and the like. It is a novel, inventive molecule that exhibits industrial applicability both when present in a plant cell or in a plant genome, and when present outside of a plant cell, and therefore, exhibits and is intended to exhibit such utility regardless of where the molecule is located.

Reference in this disclosure to an “isolated protein molecule” or an equivalent term or phrase is intended to mean that the protein molecule is one that is present alone or in combination with other compositions but not within its natural environment. For example, the insecticidal protein molecules expressed by soybean event GM_CSM63770 are not naturally found in native soybean plant samples. The Cry1A.2 and Cry1B.2 insecticidal proteins of soybean event GM_CSM63770 are isolated protein molecules so long as the insecticidal proteins or variants thereof was not within the protein of the bacterium from which the protein is naturally found.

The DNA sequence of the region spanning the connection by phosphodiester bond linkage of one end of the transgenic insert to the flanking soybean genomic DNA is referred to as a “junction.” A junction is the connection point of the transgenic insert and flanking DNA as one contiguous molecule. One junction is found at the 5′ end of the transgenic insert and the other is found at the 3′ end of the transgenic insert, referred to herein as the 5′ and 3′ junction, respectively. A “junction sequence” refers to a DNA sequence of any length that spans the 5′ or 3′ junction of an event. Junction sequences of soybean event GM_CSM63770 are apparent to one of skill in the art using SEQ ID NO:10. Examples of junction sequences of GM_CSM63770 are provided as SEQ ID NOs:1-8. FIG. 1 illustrates the physical arrangement of the junction sequences, arranged from 5′ to 3′, relative to SEQ ID NO:10. The junction sequences of GM_CSM63770 may be present as part of the genome of a plant, seed, or cell containing GM_CSM63770. The identification of any one or more of the junction sequences in a sample from a plant, plant part, seed, or cell indicates that the DNA was obtained from soybean containing GM_CSM63770 and is diagnostic for, or characteristic of, the presence of soybean event GM_CSM63770.

The junction sequences for soybean event GM_CSM63770 may be represented by a sequence from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10. For example, the junction sequences may be arbitrarily represented by the nucleotide sequences provided as SEQ ID NO:1 (5′ junction sequence) and SEQ ID NO:2 (3′ junction sequence). Alternatively, the junction sequences may be arbitrarily represented by the nucleotide sequences provided as SEQ ID NO:3 (5′ junction sequence) and SEQ ID NO:4 (3′ junction sequence). Alternatively, the junction sequences may be arbitrarily represented by the nucleotide sequences provided as SEQ ID NO:5 (5′ junction sequence) and SEQ ID NO:6 (3′ junction sequence). Alternatively, the junction sequences may be arbitrarily represented by the nucleotide sequences provided as SEQ ID NO:7 (5′ junction sequence) and SEQ ID NO:8 (3′ junction sequence). These nucleotide sequences are connected by phosphodiester linkage, and in soybean event GM_CSM63770 are present as part of the recombinant plant cell genome.

These junction sequences are diagnostic for, or characteristic of, the presence of soybean event GM_CSM63770, or the construct comprised therein. Thus, the identification of one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 in a sample derived from a soybean plant, soybean seed, or soybean plant part is diagnostic that the DNA was obtained from soybean event GM_CSM63770. The invention thus provides a DNA molecule that contains at least one of the nucleotide sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Any segment of DNA derived from transgenic soybean event GM_CSM63770 that is sufficient to include at least one of the sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 is within the scope of the invention. In addition, any polynucleotide comprising a sequence complementary to any of the sequences described within this paragraph is within the scope of the invention.

The invention provides exemplary DNA molecules that can be used either as primers or probes for detecting the presence of DNA derived from a soybean plant comprising event GM_CSM63770 DNA in a sample. Such primers or probes are specific for a target nucleic acid sequence and, as such, are useful for the identification of soybean event GM_CSM63770 nucleic acid sequence by the methods of the invention described herein.

It is intended by use of the word “derived” that a particular DNA molecule is in the soybean plant genome or is capable of being detected in soybean plant DNA. “Capable of being detected” refers to the ability of a particular DNA segment to be amplified and its size or sequence characterized or elucidated by DNA sequence analysis, i.e., the target DNA segment, and the subsequent ability to detect the binding of the probe to the target. The particular DNA segment or target DNA segment of the present invention is present within soybean that contains the insertion soybean event GM_CSM63770.

A “probe” is a nucleic acid molecule that is complementary to a strand of target nucleic acid and is useful in hybridization methods. A probe may be attached a conventional detectable label or reporter molecule, e.g., a radioactive isotope, ligand, chemiluminescent agent, or enzyme. Such a probe is complementary to a strand of a target nucleic acid and, in the case of the present invention, to a strand of DNA from GM_CSM63770 whether from a GM_CSM63770 containing plant or from a sample that includes GM_CSM63770 DNA. Thus, the probes for use herein may comprise DNA molecules or polynucleotide segments of sufficient length to function under stringent hybridization conditions as defined herein to bind to a particular unique segment of DNA present within and diagnostic for, or characteristic of, event GM_CSM63770 in a sample. Such a probe can be designed to bind only to a single junction or other novel sequence present only in the soybean event GM_CSM63770, or two or more such single junction segments. Probes according to the present invention include not only deoxyribonucleic or ribonucleic acids, but also polyamides and other probe materials that bind specifically to a target DNA sequence and can be used to detect the presence of that target DNA sequence. An exemplary DNA sequence useful as a probe for detecting soybean event GM_CSM63770 is provided as SEQ ID NO:16 (PB4832).

A “primer” is typically a DNA molecule that is designed for use in specific annealing or hybridization methods that involve thermal amplification. Primers may comprise pairs of different oligonucleotides or polynucleotide segments for use in a thermal amplification reaction which amplifies a particular DNA target segment. Each primer in the pair is designed to bind to a rather specific segment of DNA within or near a segment DNA of interest for amplification. The primers bind in such a way that these then act as localized regions of nucleic acid sequence polymerization resulting in the production of one or more amplicons (amplified target segments of DNA). The amplicon produced from such reaction would have a DNA sequence corresponding to sequence of the template DNA located between the two sites where the primers hybridized to the template. In certain embodiments, use of primers designed to bind to unique segments of soybean event GM_CSM63770 and that amplify particular amplicons containing one or more of the junction sequences described herein, and the detection and/or characterization of such amplicons upon completion or termination of polymerase reaction, is diagnostic for, or characteristic of, the presence of soybean event GM_CSM63770 in a particular sample. The skilled artisan is well familiar with this amplification method and no recitation of the specifics of amplification is necessary here.

A primer is typically designed to hybridize to a complementary target DNA strand to form a hybrid between the primer and target DNA strand, and the presence of the primer is a point of recognition by a polymerase to begin extension of the primer (i.e., polymerization of additional nucleotides into a lengthening nucleotide molecule) using as a template the target DNA strand. Primer pairs refer to use of two primers binding opposite strands of a double stranded nucleotide segment for the purpose of amplifying linearly the polynucleotide segment between the positions targeted for binding by the individual members of the primer pair, typically in a thermal amplification reaction or other conventional nucleic-acid amplification methods. Exemplary DNA molecules useful as primers are provided as SEQ ID NO:14, SEQ ID NO:15, SEQ ID NO:17, SEQ ID NO:18, and SEQ ID NO:20.

The primer pair SEQ ID NO:14 and SEQ ID NO:15 are useful as a first DNA molecule and a second DNA molecule that is different from the first DNA molecule, and both are each of sufficient length of contiguous nucleotides of SEQ ID NO:10 to function as DNA primers that, when used together in a thermal amplification reaction with template DNA derived from soybean event GM_CSM63770, to produce an amplicon diagnostic for, or characteristic of, soybean event GM_CSM63770 DNA in a sample. The primer pair SEQ ID NO:17 and SEQ ID NO:18 are useful as a first DNA molecule and a second DNA molecule that is different from the first DNA molecule, and both are each of sufficient length of contiguous nucleotides of a locus within the soybean genome to function as DNA primers that, when used together in a thermal amplification reaction with template DNA derived from soybean event GM_CSM63770, to produce an amplicon that serves as an internal control for both the diagnosis of soybean event GM_CSM63770, as well as the zygosity of soybean event GM_CSM63770 DNA in a sample. The primer pair SEQ ID NO:20 and SEQ ID NO:15 are useful as a first DNA molecule and a second DNA molecule that is different from the first DNA molecule, and both are each of sufficient length of contiguous nucleotides of a locus within the soybean genome to function as DNA primers that, when used together in a thermal amplification reaction with template DNA derived from soybean event GM_CSM63770, to produce an amplicon diagnostic for, or characteristic of, non-inserted wild-type soybean genomic DNA not comprising event GM_CSM63770. It is within the skill of the art to determine for any particular desired amplification parameters, which probes and primers would be optimum for inclusion in the thermal amplification reaction to detect the presence or absence of the transgenic event DNA of the present invention based on the DNA sequences provided in the inserted DNA (SEQ ID NO:9) and the full segment of DNA set forth herein as SEQ ID NO:10 which defines the transgenic soybean event of this invention, GM_CSM63370.

DNA probes and DNA primers are generally eleven (11) polynucleotides or more in length, often eighteen (18) polynucleotides or more, twenty-four (24) polynucleotides or more, or thirty (30) polynucleotides or more. Such probes and primers are selected to be of sufficient length to hybridize specifically to a target sequence under high stringency hybridization conditions. Preferably, probes and primers according to the present invention have complete sequence similarity with the target sequence, although probes differing from the target sequence that retain the ability to hybridize to target sequences may be designed by conventional methods.

The nucleic acid probes and primers of the present invention hybridize under stringent conditions to a target DNA molecule. Any conventional nucleic acid hybridization or amplification method can be used to identify the presence of DNA from a transgenic plant in a sample. Polynucleic acid molecules also referred to as nucleic acid segments or fragments thereof are capable of specifically hybridizing to other nucleic acid molecules under certain circumstances.

As used herein, two polynucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989, and by Haymes et al., In: Nucleic Acid Hybridization, A Practical Approach, IRL Press, Washington, DC (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. Appropriate stringency conditions that promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. In a preferred embodiment, a polynucleic acid of the present invention will specifically hybridize to one or more of the nucleic acid molecules set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, or complements thereof or fragments of either under moderately stringent conditions, for example at about 2.0×SSC and about 65° C. In a particularly preferred embodiment, a nucleic acid of the present invention will specifically hybridize to one or more of the nucleic acid molecules set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, or complements or fragments of either under high stringency conditions. In one aspect of the present invention, a preferred marker nucleic acid molecule of the present invention has the nucleic acid sequence set forth in SEQ ID NO:1, or SEQ ID NO:2, or SEQ ID NO:3, or SEQ ID NO:4, or SEQ ID NO:5, or SEQ ID NO:6, or SEQ ID NO:7, or SEQ ID NO:8, or SEQ ID NO:9, or SEQ ID NO:10, or complements thereof, or fragments of either. The hybridization of the probe to the target DNA molecule can be detected by any number of methods known to those skilled in the art, these can include, but are not limited to, fluorescent tags, radioactive tags, antibody-based tags, and chemiluminescent tags.

Regarding the amplification of a target nucleic acid sequence (e.g., by PCR) using a particular amplification primer pair, “stringent conditions” are conditions that permit the primer pair to hybridize only to the target nucleic acid sequence to which a primer having the corresponding wild-type sequence (or its complement) would bind and preferably to produce a unique amplification product, the amplicon, in a DNA thermal amplification reaction.

The term “specific for (a target sequence)” indicates that a probe or primer hybridizes under stringent hybridization conditions only to the target sequence in a sample comprising the target sequence.

As used herein, “amplified DNA” or “amplicon” refers to the product of polynucleic acid amplification method directed to a target polynucleic acid molecule that is part of a polynucleic acid template. For example, to determine whether a soybean plant resulting from a sexual cross contains transgenic plant genomic DNA from a soybean plant comprising event GM_CSM63770 of the present invention, DNA that is extracted from a soybean plant tissue sample may be subjected to a polynucleic acid amplification method using a primer pair that includes a first primer derived from a genomic DNA sequence in the region flanking the heterologous inserted DNA of event GM_CSM63770 and is elongated by polymerase 5′ to 3′ in the direction of the inserted DNA. The second primer is derived from the heterologous inserted DNA molecule is elongated by the polymerase 5′ to 3′ in the direction of the flanking genomic DNA from which the first primer is derived. The amplicon may range in length from the combined length of the primer pair plus one nucleotide base pair, or plus about fifty nucleotide base pairs, or plus about two hundred-fifty nucleotide base pairs, or plus about four hundred-fifty nucleotide base pairs or more. Alternatively, a primer pair can be derived from genomic sequence on both sides of the inserted heterologous DNA so as to produce an amplicon that includes the entire insert polynucleotide sequence (e.g., a forward primer isolated from the genomic portion on the 5′ end of SEQ ID NO:10 and a reverse primer isolated from the genomic portion on the 3′ end of SEQ ID NO:10 that amplifies a DNA molecule comprising the inserted DNA sequence (SEQ ID NO:9) identified herein in the event GM_CSM63770 genome). A member of a primer pair derived from the plant genomic sequence adjacent to the inserted transgenic DNA is located a distance from the inserted DNA sequence, this distance can range from one nucleotide base pair up to about twenty thousand nucleotide base pairs. The use of the term “amplicon” specifically excludes primer dimers that may be formed in the DNA thermal amplification reaction.

For practical purposes, one should design primers which produce amplicons of a limited size range, for example, between 100 to 1000 bases. Smaller (shorter polynucleotide length) sized amplicons in general are more reliably produced in thermal amplification reactions, allow for shorter cycle times, and can be easily separated and visualized on agarose gels or adapted for use in endpoint TAQMAN®-like assays. Smaller amplicons can be produced and detected by methods known in the art of DNA amplicon detection. In addition, amplicons produced using the primer pairs can be cloned into vectors, propagated, isolated, and sequenced or can be sequenced directly with methods well established in the art. Any primer pair derived from the combination of SEQ ID NO:11 and SEQ ID NO:9 or the combination of SEQ ID NO:12 and SEQ ID NO:9 that are useful in a DNA amplification method to produce an amplicon diagnostic for, or characteristic of, soybean event GM_CSM63770 or progeny thereof is an aspect of the invention. Any single isolated DNA polynucleotide primer molecule comprising at least 15 contiguous nucleotides of SEQ ID NO:11, or its complement that is useful in a DNA amplification method to produce an amplicon diagnostic for, or characteristic of, soybean event GM_CSM63770 or progeny thereof is an aspect of the invention. Any single isolated DNA polynucleotide primer molecule comprising at least 15 contiguous nucleotides of SEQ ID NO:12, or its complement that is useful in a DNA amplification method to produce an amplicon diagnostic for, or characteristic of, plants comprising soybean event GM_CSM63770 or progeny thereof is an aspect of the invention. Any single isolated DNA polynucleotide primer molecule comprising at least 15 contiguous nucleotides of SEQ ID NO:9, or its complement that is useful in a DNA amplification method to produce an amplicon diagnostic for, or characteristic of, soybean event GM_CSM63770 or progeny thereof is an aspect of the invention.

Polynucleic acid amplification can be accomplished by any of the various polynucleic acid amplification methods known in the art, including the polymerase chain reaction (PCR). Amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb (kilobase) of genomic DNA and up to 42 kb of bacteriophage DNA (Cheng et al., Proc. Natl. Acad. Sci. USA 91:5695-5699, 1994). These methods as well as other methods known in the art of DNA amplification may be used in the practice of the present invention. The sequence of the heterologous DNA insert or flanking genomic DNA sequence from soybean event GM_CSM63770 can be verified (and corrected if necessary) by amplifying such DNA molecules from soybean seed containing event GM_CSM63770 DNA or soybean plants grown from the soybean seed containing event GM_CSM63770 DNA deposited with the ATCC having accession No. PTA-126048, using primers derived from the sequences provided herein, followed by standard DNA sequencing of the PCR amplicon or cloned DNA fragments thereof.

The diagnostic amplicon produced by these methods may be detected by a plurality of techniques. One such method is Genetic Bit Analysis (Nikiforov, et al. Nucleic Acid Res. 22:4167-4175, 1994) where a DNA oligonucleotide is designed that overlaps both the adjacent flanking genomic DNA sequence and the inserted DNA sequence. The oligonucleotide is immobilized in wells of a microtiter plate. Following PCR of the region of interest (using one primer in the inserted sequence and one in the adjacent flanking genomic sequence), a single-stranded PCR product can be hybridized to the immobilized oligonucleotide and serve as a template for a single base extension reaction using a DNA polymerase and labeled dideoxynucleotide triphosphates (ddNTPs) specific for the expected next base. Readout may be fluorescent or ELISA-based. A signal indicates presence of the transgene/genomic sequence due to successful amplification, hybridization, and single base extension.

Another method is the Pyrosequencing technique as described by Winge (Innov. Pharma. Tech. 00:18-24, 2000). In this method, an oligonucleotide is designed that overlaps the adjacent genomic DNA and insert DNA junction. The oligonucleotide is hybridized to single-stranded PCR product from the region of interest (one primer in the inserted sequence and one in the flanking genomic sequence) and incubated in the presence of a DNA polymerase, ATP, sulfurylase, luciferase, apyrase, adenosine 5′ phosphosulfate and luciferin. dNTPs are added individually and the enzymatic reaction of luciferase with these reagents and substrates results in is the release of photons (a signal of light) which are then measured or observed. A light signal indicates the presence of the transgene/genomic sequence due to successful amplification, hybridization, and single or multi-base extension.

Fluorescence Polarization as described by Chen, et al., (Genome Res. 9:492-498, 1999) is a method that can be used to detect the amplicon of the present invention. Using this method an oligonucleotide is designed that overlaps the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene/genomic sequence due to successful amplification, hybridization, and single base extension.

Real-time Polymerase Chain Reaction (PCR) is the ability to monitor the progress of the PCR as it occurs (i.e., in real time). Data is collected throughout the PCR process, rather than at the end of the PCR. In real-time PCR, reactions are characterized by the point in time during cycling when amplification of a target is first detected rather than the amount of target accumulated after a fixed number of cycles. In a real-time PCR assay, a positive reaction is detected by accumulation of a fluorescent signal. The higher the starting copy number of the nucleic acid target, the sooner a significant increase in fluorescence is observed. The cycle threshold (Ct value) is defined as the number of cycles required for the fluorescent signal to cross the threshold (i.e., exceeds background level). Ct levels are inversely proportional to the amount of target nucleic acid in the sample (i.e., the lower the Ct value, the greater the amount of target nucleic acid in the sample).

Taqman® (PE Applied Biosystems, Foster City, CA) is described as a method of detecting and quantifying the presence of a DNA sequence using real-time PCR and is fully understood in the instructions provided by the manufacturer. Briefly, a FRET oligonucleotide probe is designed that overlaps the genomic flanking and insert DNA junction. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the transgene/genomic sequence due to successful amplification and hybridization.

Molecular Beacons have been described for use in sequence detection as described in Tyangi, et al. (Nature Biotech. 14:303-308, 1996). Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermalstable polymerase and dNTPs. Following successful PCR amplification, hybridization of the FRET probe to the target sequence results in the removal of the probe secondary structure and spatial separation of the fluorescent and quenching moieties. A fluorescent signal results. A fluorescent signal indicates the presence of the flanking/transgene insert sequence due to successful amplification and hybridization.

DNA detection kits that are based on DNA amplification methods contain DNA primer molecules that hybridize specifically to a target DNA and amplify a diagnostic amplicon under the appropriate reaction conditions. The kit may provide an agarose gel-based detection method or any number of methods of detecting the diagnostic amplicon that are known in the art. DNA detection kits can be developed using the compositions disclosed herein and are useful for identification of soybean event GM_CSM63770 DNA in a sample and can be applied to methods for breeding soybean plants containing soybean event GM_CSM63770 DNA. A kit that contains DNA primers that are homologous or complementary to any portion of the soybean genomic region as set forth in SEQ ID NO:10 and to any portion of the inserted transgenic DNA as set forth in SEQ ID NO:10 is an object of the invention. The DNA molecules can be used in DNA amplification methods (PCR) or as probes in polynucleic acid hybridization methods, i.e., southern analysis, northern analysis.

Probes and primers according to the invention may have complete sequence identity with the target sequence, although primers and probes differing from the target sequence that retain the ability to hybridize preferentially to target sequences may be designed by conventional methods. In order for a nucleic acid molecule to serve as a primer or probe it need only be sufficiently complementary in sequence to be able to form a stable double-stranded structure under the particular solvent and salt concentrations employed. Any conventional nucleic acid hybridization or amplification method can be used to identify the presence of transgenic DNA from soybean event GM_CSM63770 in a sample. Probes and primers are generally at least about 11 nucleotides, at least about 18 nucleotides, at least about 24 nucleotides, or at least about 30 nucleotides or more in length. Such probes and primers hybridize specifically to a target DNA sequence under stringent hybridization conditions. Conventional stringency conditions are described by Sambrook et al., 1989, and by Haymes et al., In: Nucleic Acid Hybridization, A Practical Approach, IRL Press, Washington, DC (1985).

Any number of methods well known to those skilled in the art can be used to isolate and manipulate a DNA molecule, or fragment thereof, disclosed in the invention, including thermal amplification methods. DNA molecules, or fragments thereof, can also be obtained by other techniques such as by directly synthesizing the fragment by chemical means, as is commonly practiced by using an automated oligonucleotide synthesizer.

The DNA molecules and corresponding nucleotide sequences provided herein are therefore useful for, among other things, identifying soybean event GM_CSM63770, detecting the presence of DNA derived from the transgenic soybean event GM_CSM63770 in a sample, and monitoring samples for the presence and/or absence of soybean event GM_CSM63770 or plant parts derived from soybean plants comprising soybean event GM_CSM63770.

By reference to soybean it is intended that soybean plants, soybean plant cells, soybean seed, soybean pollen and ova, soybean plant parts, soybean progeny plants, and soybean commodity products are within the scope of the invention, so long as each embodiment contains a detectable amount of DNA corresponding to any one, two, or more of the segments described herein as being diagnostic for, or characteristic of, the presence of soybean event GM_CSM63770 (e.g., such as a polynucleotide having at least one of the sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10). Soybean plants, plant cells, seed, pollen and or ova, plant parts, and progeny plants of the invention may also contain one or more additional transgenes. Such additional transgene(s) may be any nucleotide sequence encoding a protein or RNA molecule conferring a desirable trait including but not limited to increased insect resistance, increased water use efficiency, increased yield performance, increased drought resistance, increased seed quality, and/or increased herbicide tolerance.

The invention provides soybean plants, soybean plant cells, soybean seed, soybean plant parts (such as pollen, ovule, silk, spike, anther, cob, root tissue, stalk tissue, leaf tissue), soybean progeny plants derived from a transgenic soybean plant containing GM_CSM63770 DNA. A representative sample of soybean seed containing soybean event GM_CSM63770 DNA has been deposited according to the Budapest Treaty with the American Type Culture Collection (ATCC®). The ATCC repository has assigned the Patent Deposit Designation PTA-126048 to the seed containing soybean event GM_CSM63770 DNA.

The invention provides soybean plants, plant cells, plant parts and plant seed comprising a recombinant DNA construct integrated in chromosome 19, wherein the recombinant DNA construct confers resistance against Lepidopteran insect pest species. The recombinant DNA construct is integrated in a position of said chromosome flanked by at least 50 contiguous nucleotides of SEQ ID NO: 11 or 36 and 50 contiguous nucleotides of SEQ ID NO:12 or 37. The at least 50 contiguous nucleotides of SEQ ID NO:11 or 36 can comprise one or more nucleotide sequences selected from SEQ ID NOs:38-137. The at least nucleotides of SEQ ID NO:12 or 37 can comprise one or more nucleotide sequences selected from SEQ ID NOs:138-237.

The invention provides a microorganism comprising a DNA molecule having at least one sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 present in its genome. An example of such a microorganism is a transgenic plant cell.

Microorganisms, such as a plant cell of the invention, are useful in many industrial applications, including but not limited to: (i) use as research tool for scientific inquiry or industrial research; (ii) use in culture for producing endogenous or recombinant carbohydrate, lipid, nucleic acid, or protein products or small molecules that may be used for subsequent scientific research or as industrial products; and (iii) use with modern plant tissue culture techniques to produce transgenic plants or plant tissue cultures that may then be used for agricultural research or production. The production and use of microorganisms such as transgenic plant cells utilizes modern microbiological techniques and human intervention to produce a man-made, unique microorganism. In this process, recombinant DNA is inserted into a plant cell's genome to create a transgenic plant cell that is separate and unique from naturally occurring plant cells. This transgenic plant cell can then be cultured much like bacteria and yeast cells using modern microbiology techniques and may exist in an undifferentiated, unicellular state. The transgenic plant cell's new genetic composition and phenotype is a technical effect created by the integration of the heterologous DNA into the genome of the cell. Another aspect of the invention is a method of using a microorganism of the invention. Methods of using microorganisms of the invention, such as transgenic plant cells, include (i) methods of producing transgenic cells by integrating recombinant DNA into the genome of the cell and then using this cell to derive additional cells possessing the same heterologous DNA; (ii) methods of culturing cells that contain recombinant DNA using modern microbiology techniques; (iii) methods of producing and purifying endogenous or recombinant carbohydrate, lipid, nucleic acid, or protein products from cultured cells; and (iv) methods of using modern plant tissue culture techniques with transgenic plant cells to produce transgenic plants or transgenic plant tissue cultures.

Plants of the invention may pass along the soybean event GM_CSM63770 DNA, including transgene inserted in soybean event GM_CSM63770, to progeny, typically through conventional breeding and selection. As used herein, “progeny” includes any plant, plant cell, seed, and/or regenerable plant part containing the event GM_CSM63770 DNA derived from an ancestor plant and/or comprising a DNA molecule having at least one sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Plants, progeny, and seed may be homozygous or heterozygous for the transgene of event GM_CSM63770. Progeny may be grown from seed produced by a soybean event GM_CSM63770 containing plant and/or from seed produced by a plant fertilized with pollen from a soybean event GM_CSM63770 containing plant.

Progeny plants may be self-pollinated (also known as “selfing”) to generate a true breeding line of plants, i.e., plants homozygous for the transgene. Selfing of appropriate progeny can produce plants that are homozygous for both added exogenous genes.

Alternatively, progeny plants may be out-crossed, e.g., bred with another unrelated plant, to produce a varietal or a hybrid seed or plant. The other unrelated plant may be transgenic or non-transgenic. A varietal or hybrid seed or plant of the invention may thus be derived by sexually crossing a first parent that lacks the specific and unique DNA of the soybean event GM_CSM63770 with a second parent comprising soybean event GM_CSM63770, resulting in a hybrid comprising the specific and unique DNA of the soybean event GM_CSM63770. Each parent can be a hybrid or an inbred/varietal, so long as the cross or breeding results in a plant or seed of the invention, i.e., a seed having at least one allele containing the DNA of soybean event GM_CSM63770 and/or a DNA molecule having at least one sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Two different transgenic plants may thus be crossed to produce hybrid offspring that contain two independently segregating, added, exogenous genes. For example, soybean event GM_CSM63770 containing Cry1A.2 and Cry1B.2 conferring insect resistance to soybean can be crossed with other transgenic soybean plants to produce a plant having the characteristics of both transgenic parents. One example of this would be a cross of soybean event GM_CSM63770 containing Cry1A.2 and Cry1B.2, conferring Lepidopteran resistance to soybean with a plant having one or more additional traits such as herbicide tolerance, insect resistance, or drought tolerance, resulting in a progeny plant or seed that has resistance to Lepidopteran insect pests and has at least one or more additional traits. Back-crossing to a parental plant and out-crossing with a non-transgenic plant are also contemplated, as is vegetative propagation. Descriptions of other breeding methods that are commonly used for different traits and crops can be found in one of several references, e.g., Fehr, in Breeding Methods for Cultivar Development, Wilcox J. ed., American Society of Agronomy, Madison WI (1987).

Plants, progeny, seed, pollen and ova, cells and plant parts of the invention may also contain one or more additional soybean trait(s) or transgenic events, particularly those introduced by crossing a soybean plant containing soybean event GM_CSM63770 with another soybean plant containing the additional trait(s) or transgenic events. Such trait(s) or transgenic events include, but are not limited to, increased insect resistance, herbicide tolerance, increased water use efficiency, increased yield performance, increased drought resistance, increased seed quality, improved nutritional quality, hybrid seed production, or disease or fungal resistance. Soybean transgenic events are known to those of skill in the art. For example, a list of such traits is provided by the United States Department of Agriculture's (USDA) Animal and Plant Health Inspection Service (APHIS) and can be found on their website www.aphis.usda.gov on the worldwide web. Two or more transgenic events may thus be combined in a progeny seed or plant by crossing two parent plants each comprising one or more transgenic events, collecting the progeny seed, and selecting for progeny seed or plants that contain the two or more transgenic events. These steps may be repeated until the desired combination of transgenic events in a progeny is achieved. Back-crossing to a parental plant and out-crossing with a non-transgenic plant are also contemplated and is vegetative propagation.

A plant part that is derived from soybean plants comprising soybean event GM_CSM63770 is also provided. As used herein, a “plant part” refers to any part of a plant which is comprised of material derived from a soybean plant comprising event GM_CSM63770. Plant parts include but are not limited to pollen, ovule, pod, flower, root or stem tissue, fibers, and leaves. Plant parts may be viable, nonviable, regenerable, and/or nonregenerable.

Further provided is a commodity product that is derived from soybean plants comprising soybean event GM_CSM63770 and that contains a detectable amount of a nucleic acid specific for soybean event GM_CSM63770. As used herein, a “commodity product” refers to any composition or product which is comprised of material derived from a soybean plant, whole or processed soybean seed, or one or more plant cells and/or plant parts containing the soybean event GM_CSM63770 DNA. Nonviable commodity products include but are not limited to nonviable seed, whole or processed seed, seed parts, and plant parts; animal feed comprising soybean, soybean oil, soybean protein, soybean meal, soybean flour, soybean flakes, soybean bran, soybean milk, soybean cheese, soybean wine, paper comprising soybean, cream comprising soybean, soybean biomass, and fuel products produced using soybean plants and soybean parts. Viable commodity products include but are not limited to seed, plants, and plant cells. The soybean plants comprising soybean event GM_CSM63770 can thus be used to manufacture any commodity product typically acquired from soybean. Any such commodity product that is derived from soybean plants comprising soybean event GM_CSM63770 may contain at least a detectable amount of the specific and unique DNA corresponding to soybean event GM_CSM63770, and specifically may contain a detectable amount of a polynucleotide comprising a DNA molecule having at least one sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Any standard method of detection for nucleotide molecules may be used, including methods of detection disclosed herein. A commodity product is with the scope of the invention if there is any detectable amount of a DNA molecule having at least one sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 in the commodity product.

The soybean plants, soybean plant cells, soybean seed, soybean plant parts (such as pollen, ovule, silk, spike, anther, cob, root tissue, stalk tissue, leaf tissue), soybean progeny plants, and commodity products of the invention are therefore, useful for, among other things, growing plants for the purpose of producing seed and/or plant parts comprising soybean event GM_CSM63770 for agricultural purposes, producing progeny comprising soybean event GM_CSM63770 for plant breeding and research purposes, use with microbiological techniques for industrial and research applications, and sale to consumers.

Methods for producing an insect resistant soybean plant comprising the DNA sequences specific and unique to event GM_CSM63770 of the invention are provided. Transgenic plants used in these methods may be homozygous or heterozygous for the transgene. Progeny plants produced by these methods may be varietal or hybrid plants; may be grown from seed produced by soybean event GM_CSM63770 containing plant and/or from seed produced by a plant fertilized with pollen from a soybean event GM_CSM63770 containing plant; and may be homozygous or heterozygous for the transgene. Progeny plants may be subsequently self-pollinated to generate a true breeding line of plants, i.e., plants homozygous for the transgene, or alternatively may be out-crossed, e.g., bred with another unrelated plant, to produce a varietal or a hybrid seed or plant.

Methods of detecting the presence of DNA derived from a soybean cell, soybean tissue, soybean seed, or soybean plant comprising soybean event GM_CSM63770 in a sample are provided. One method comprises (i) extracting a DNA sample from at least one soybean cell, soybean tissue, soybean seed, or soybean plant; (ii) contacting the DNA sample with at least one primer that is capable of producing DNA sequence specific to event GM_CSM63770 DNA under conditions appropriate for DNA sequencing; (iii) performing a DNA sequencing reaction; and then (iv) confirming that the nucleotide sequence comprises a nucleotide sequence specific for event GM_CSM63770, of the construct comprised therein, such as one selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Another method comprises (i) extracting a DNA sample from at least one soybean cell, soybean tissue, soybean seed, or soybean plant; (ii) contacting the DNA sample with a primer pair that is capable of producing an amplicon from event GM_CSM63770 DNA under conditions appropriate for DNA amplification; (iii) performing a DNA amplification reaction; and then (iv) detecting the amplicon molecule and/or confirming that the nucleotide sequence of the amplicon comprises a nucleotide sequence specific for event GM_CSM63770, such as one selected from the group consisting of SEQ ID NO:1, SEQ ID NO:2, SEQ ID NO:3, SEQ ID NO:4, SEQ ID NO:5, and SEQ ID NO:6. The amplicon should be one that is specific for event GM_CSM63770, such as an amplicon that comprises SEQ ID NO:1, or SEQ ID NO:2, or SEQ ID NO:3, or SEQ ID NO:4, or SEQ ID NO:5, or SEQ ID NO:6. The detection of a nucleotide sequence specific for soybean event GM_CSM63770 in the amplicon is determinative and/or diagnostic for, or characteristic of, the presence of the soybean event GM_CSM63770 specific DNA in the sample. An example of a primer pair that is capable of producing an amplicon from event GM_CSM63770 DNA under conditions appropriate for DNA amplification is provided as SEQ ID NO:14 and SEQ ID NO:15. Other primer pairs may be readily designed by one of skill in the art and would produce an amplicon comprising SEQ ID NO:1, or SEQ ID NO:2, or SEQ ID NO:3, or SEQ ID NO:4, or SEQ ID NO:5, or SEQ ID NO:6, wherein such a primer pair comprises at least on primer within the genomic region flanking the insert and a second primer within the insert. Another method of detecting the presence of DNA derived from a soybean cell, soybean tissue, soybean seed, or soybean plant comprising soybean event GM_CSM63770 in a sample comprises (i) extracting a DNA sample from at least one soybean cell, soybean tissue, soybean seed, or soybean plant; (ii) contacting the DNA sample with a DNA probe specific for event GM_CSM63770 DNA; (iii) allowing the probe and the DNA sample to hybridize under stringent hybridization conditions; and then (iv) detecting hybridization between the probe and the target DNA sample. An example of the sequence of a DNA probe that is specific for event GM_CSM63770 is provided as SEQ ID NO:16. Other probes may be readily designed by one of skill in the art and would comprise at least one fragment of genomic DNA flanking the insert and at least one fragment of insert DNA such as sequence provided in, SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10. Detection of probe hybridization to the DNA sample is diagnostic for, or characteristic of, the presence of soybean event GM_CSM63770 specific DNA in the sample. Absence of hybridization is alternatively diagnostic for, or characteristic of, the absence of soybean event GM_CSM63770 specific DNA in the sample.

DNA detection kits are provided that are useful for the identification of soybean event GM_CSM63770 DNA in a sample and can also be applied to methods for breeding soybean plants containing the appropriate event DNA. Such kits contain DNA primers and/or probes comprising fragments of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. One example of such a kit comprises at least one DNA molecule of sufficient length of continuous nucleotides of SEQ ID NO:10 to function as a DNA probe useful for detecting the presence and/or absence of DNA derived from transgenic soybean plants comprising event GM_CSM63770 in a sample. The DNA derived from transgenic soybean plants comprising event GM_CSM63770 would comprise a DNA molecule having at least one sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. A DNA molecule sufficient for use as a DNA probe is provided that is useful for determining, detecting, or diagnosing the presence and/or absence of soybean event GM_CSM63770 DNA in a sample is provided as SEQ ID NO: 16. Other probes may be readily designed by one of skill in the art and should comprise at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 contiguous nucleotides of SEQ ID NO:10 and be sufficiently unique to soybean event GM_CSM63770 DNA in order to identify DNA derived from the event.

Another type of kit comprises a primer pair useful for producing an amplicon useful for detecting the presence and/or absence of DNA derived from transgenic soybean event GM_CSM63770 in a sample. Such a kit would employ a method comprising contacting a target DNA sample with a primer pair as described herein, then performing a nucleic acid amplification reaction sufficient to produce an amplicon comprising a DNA molecule having at least one sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 and then detecting the presence and/or absence of the amplicon. Such a method may also include sequencing the amplicon or a fragment thereof, which would be determinative of, i.e., diagnostic for, or characteristic of, the presence of the soybean event GM_CSM63770 specific DNA in the target DNA sample. Other primer pairs may be readily designed by one of skill in the art and should comprise at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of sequences provided in, but not limited to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, and be sufficiently unique to soybean event GM_CSM63770 DNA in order to identify DNA derived from the event.

The kits and detection methods of the invention are useful for, among other things, identifying soybean event GM_CSM63770, selecting plant varieties or hybrids comprising soybean event GM_CSM63770, detecting the presence of DNA derived from the transgenic soybean plant comprising event GM_CSM63770 in a sample, and monitoring samples for the presence and/or absence of soybean plants comprising event GM_CSM63770, or plant parts derived from soybean plants comprising event GM_CSM63770.

The sequences of the heterologous DNA insert, junction sequences, or flanking sequence from soybean event GM_CSM63770 can be verified (and corrected if necessary) by amplifying such sequences from the event using primers derived from the sequences provided herein followed by standard DNA sequencing of the amplicon or of the cloned DNA.

Methods of detecting the zygosity of the transgene allele of DNA derived from a soybean cell, soybean tissue, soybean seed, or soybean plant comprising soybean event GM_CSM63770 in a sample are provided. One method comprises (i) extracting a DNA sample from at least one soybean cell, soybean tissue, soybean seed, or soybean plant; (ii) contacting the DNA sample with a primer pair that is capable of producing a first amplicon diagnostic for, or characteristic of, soybean event GM_CSM63770; (iii) contacting the DNA sample with a primer pair that is capable of producing a second amplicon diagnostic for, or characteristic of, native soybean genomic DNA not comprising soybean event GM_CSM63770; (iv) performing a DNA amplification reaction; and then (v) detecting the amplicons, wherein the presence of only the first amplicon is diagnostic of a homozygous soybean event GM_CSM63770 DNA in the sample, and the presence of both the first amplicon and the second amplicon is diagnostic of a soybean plant heterozygous for soybean event GM_CSM63770 allele. An exemplary set of primers pairs are presented as SEQ ID NO:14 and SEQ ID NO:15 which produce an amplicon diagnostic for, or characteristic of, event GM_CSM63770; and SEQ ID NO:20 and SEQ ID NO:15 which produces an amplicon diagnostic for, or characteristic of, the wild-type soybean genomic DNA not comprising soybean event GM_CSM63770. A set of probes can also be incorporated into such an amplification method to be used in a real-time PCR format using the primer pair sets described above. An exemplary set of probes are presented as SEQ ID NO:16 (diagnostic for, or characteristic of, the amplicon for the soybean event GM_CSM63770) and SEQ ID NO:21 (diagnostic for, or characteristic of, the amplicon for wild-type soybean genomic DNA not comprising soybean event GM_CSM63770).

Another method for determining zygosity comprises (i) extracting a DNA sample from at least one soybean cell, soybean tissue, soybean seed, or soybean plant; (ii) contacting the DNA sample with a probe set which contains at least a first probe that specifically hybridizes to event GM_CSM63770 DNA and at least a second probe that specifically hybridizes to soybean genomic DNA that was disrupted by insertion of the heterologous DNA of soybean event GM_CSM63770 and does not hybridize to soybean event GM_CSM63770 DNA; (iii) hybridizing the probe set with the sample under stringent hybridization conditions, wherein detecting hybridization of only the first probe under the hybridization conditions is diagnostic for, or characteristic of, a homozygous allele of soybean event GM_CSM63770 DNA in the sample; and wherein detecting hybridization of both the first probe and the second probe under the hybridization conditions is diagnostic for, or characteristic of, a heterozygous allele of soybean event GM_CSM63770 in a DNA sample.

Yet another method for determining zygosity comprises (i) extracting a DNA sample from at least one soybean cell, soybean tissue, soybean seed, or soybean plant; (ii) contacting the DNA sample with a primer pair that is capable of producing an amplicon diagnostic for, or characteristic of, the allele of soybean vent GM_CSM63770; (iii) contacting the DNA sample with a primer pair that is capable of producing an amplicon of an internal standard known to be single-copy and homozygous in the soybean plant; (iv) contacting the DNA sample with a probe set which contains at least a first probe that specifically hybridizes to the allele of event GM_CSM63770, and at least a second probe that specifically hybridizes to the internal standard genomic DNA known to be single-copy and homozygous in the soybean plant; (v) performing a DNA amplification reaction using real-time PCR and determining the cycle thresholds (Ct values) of the amplicon corresponding to the toxin coding sequence and the single-copy, homozygous internal standard; (vi) calculating the difference (ΔCt) between the Ct value of the single-copy, homozygous internal standard amplicon and the Ct value of the toxin coding sequence amplicon; and (vii) determining zygosity, wherein a ΔCt of around zero (0) indicates homozygosity of the inserted T-DNA and a ΔCt of around one (1) indicates heterozygosity of the inserted T-DNA. Heterozygous and homozygous events are differentiated by a ΔCt value unit of approximately one (1). Given the normal variability observed in real-time PCR due to multiple factors such as amplification efficiency and ideal annealing temperatures, the range of “about one (1)” is defined as a ΔCt of 0.75 to 1.25. Primer pairs and probes for the above method for determining zygosity can amplify and detect amplicons from the allele of event GM_CSM63770 and internal standard. Exemplary primer pairs for the detection of the amplicons corresponding to the allele of event GM_CSM63770 and internal standard are presented as SEQ ID NO:14 combined with SEQ ID NO:15 (allele of event GM_CSM63770) and SEQ ID NO:17 combined with SEQ ID NO:18 (internal standard). The accompanying exemplary probes are presented as SEQ ID NO:16 (allele of event GM_CSM63770) and SEQ ID NO:19 (internal standard).

### Deposit Information

A deposit of a representative sample of soybean seed containing event GM_CSM63770 was made on Aug. 21, 2019, according to the Budapest Treaty with the American Type Culture Collection (ATCC) having an address at 10801 University Boulevard, Manassas, Virginia USA, Zip Code 20110, and assigned ATCC Accession No. PTA-126048. Access to the deposits will be available during the pendency of the application to the Commissioner of Patents and Trademarks and persons determined by the Commissioner to be entitled thereto upon request. Upon issuance of the patent, all restrictions upon availability to the public will be irrevocably removed. The deposit will be maintained in the depository for a period of thirty (30) years, or five (5) years after the last request, or for the effective life of the patent, whichever is longer, and will be replaced as necessary during that period.

### EXAMPLES

The following Examples are included to more fully describe the invention. Summarized are the construction and testing of 125 constructs, the production of 3,343 events, and the analysis of hundreds of thousands individual plants over 6 years through the rigorous molecular, agronomic, and field testing required for the creation and selection of soybean event GM_CSM63770.

Those of skill in the art should, in light of the present disclosure, appreciate that many changes can be made in the specific embodiments that are disclosed and still obtain a like or similar result without departing from the spirit and scope of the invention.

### Example 1

### Expression Cassette Testing, Construct Design, Construct Selection, Molecular Characterization, Efficacy Testing, Field Trials, and Event Selection

It is often necessary to create and screen a large number of gene expression constructs and transformation events in order to identify a construct, and then an event, which demonstrates optimal expression of the introduced genes of interest, while also not producing agronomic or phenotypic off-types.

For these reasons, the development of a transgenic soybean plant comprising insecticidal proteins that were active against Lepidopterans without any negative effects on agronomics, yield, or stacking viability required extensive research, development, and analysis. Specifically, 6 year period, approximately 3,343 proof of concept and commercial transgenic events derived from 125 different plasmid vector constructs were developed, tested, and analyzed.

This Example describes the design and testing in soybean plants of 125 different constructs, to identify the preferred construct for event creation. Each construct varied with respect to the coding sequences for the insecticidal proteins and the transcriptional regulatory elements, and these were tested to select the preferred construct for use in expressing the insecticidal proteins in plants. Each construct had a unique configuration, varying by expression cassette composition (both insecticidal proteins and expression elements), orientation, and whether or not proteins were targeted for insertion into the chloroplast.

In an initial proof of concept and developmental stage, 121 constructs comprising different combinations of 29 distinct promoters, 1 enhancer, 16 distinct introns, and 21 distinct insect toxin coding sequences, and 10 distinct 3′ UTRs were used to generate approximately 992 transformed events. These events were evaluated for phenotypic or agronomic off-types, the level of expression of the insect toxin proteins, and efficacy against selected Lepidopteran insect pest species. The resulting efficacy and protein expression data, along with any information regarding phenotypic and agronomic off-types was used to eliminate inefficacious proteins, expression elements and combinations, and was used to design a smaller number of binary commercial transformation plasmid constructs to be used in the next phase of development.

In the next phase of development, 4 new commercial constructs were created. These constructs comprised combinations of 2 to 3 insect toxin transgene expression cassettes in different orientations (convergent or divergent). One of the constructs was eliminated prior to transformation based upon studies of the mode of action of one of the insect toxin proteins expressed in the construct. The remaining 3, pM63770, Construct-2, and Construct-3 were used to generate transformed events (also referred to as “transformants”). Table 2 below shows the event selection and elimination process for all three of the commercial construct transformed events.

TABLE 2
Event selection and elimination of stably transformed
soybean events, leading to selection of GM_CSM63770.

| | Construct pM63770 | | Construct-2 | | Construct-3 | |
|---|---|---|---|---|---|---|
| | | Total | | Total | | Total |
| Stage Analysis | Eliminated | Left | Eliminated | Left | Eliminated | Left |
| Events Plugged | 3780 | | 6911 | | 3696 | |
| Transplanted to soil | 2760 | 1020 | 6509 | 402 | 2767 | 929 |
| Discarded, didn't | 860 | 160 | 220 | 182 | 797 | 132 |
| survive | | | | | | |
| R₀ molecular and | 115 | 45 | 118 | 64 | 94 | 38 |
| efficacy | | | | | | |
| R₁ molecular | 13 | 32 | 19 | 45 | 12 | 26 |
| R₁ zygosity | 9 | 23 | 16 | 29 | 7 | 19 |
| R₁ Pre-GSS Molecular, | 13 | 10 | 8 | 21 | 19 | 0 |
| performance, maturity | | | | | | |
| R₂ GSS molecular, | 1 | 9 | 7 | 14 | | |
| lesser performer | | | | | | |
| Yield | 1 | 8 | 1 | 13 | | |
| Failed cross | 1 | 7 | 0 | 13 | | |
| Field trial, molecular, | 5 | 2 | 12 | 1 | | |
| yield | | | | | | |
| Molecular eSouthern | 1 | 1 | | | | |
| and flanking sequence | | | | | | |
| Selected Event | | GM_CSM63770 | | | | |

After shoot formation in culture, a subset of the transformed events was selected based upon visual characteristics and early molecular analysis. After transformation, 12,036 transformants were transferred to culture plates containing selective media (also referred to as “plugged”). After initial molecular characterization 9,685 were eliminated. Of the remaining 2,351 events 1,877 were eliminated based upon observations of plant health and survival. The remaining 474 R₀ events were transplanted to pots and grown in the greenhouse (GH) for further assay. The 474 events were evaluated for molecular characteristics and efficacy, and based upon these studies, 327 events were eliminated.

The remaining 147 R₀ events were allowed to self-pollinate, producing R₁ seed. The R₁ events were further characterized molecularly, assessed for zygosity, performance and maturity. From this analysis, 116 events were eliminated. At this point of selection, it was also observed that the events derived from Construct-3 did not meet the criteria for advancement, and this construct and its associated events were eliminated from further testing and analysis. The remaining 9 events derived from construct pM63770 and 14 events from Construct-2 were selected for further molecular characterization and evaluation in field studies.

During the R₂ generation, the events were further evaluated with respect to molecular characteristics and performance. During the Argentina growing season of 2017 to 2018, 23 events (9 events derived from construct pM63770 and 14 events from Construct-2) were evaluated for efficacy in screen house and field trials, and agronomics in field trials. 1 event derived from the construct pM63770 was eliminated and 7 events derived from Construct-2 were also eliminated based upon the R₂ criteria (molecular characteristics and efficacy). 1 event derived from each of the two constructs was eliminated based upon yield measurements. Another event derived from construct pM63770 was eliminated after failing to crossbreed with elite germplasm material.

The remaining 20 events, 7 derived from construct pM63770 and 13 derived from Construct-2 were assessed for efficacy in screen house and field trials and field agronomic trials. In addition, further molecular studies were performed which included identification of the locus of insertion of the T-DNA for each event, neighboring genes, and repetitive sequences near the locus of insertion. After reviewing the efficacy and agronomic data, as well as the finer molecular characterization, 5 events derived from construct pM63770 and 12 events derived from Construct-2 were eliminated, leaving only 2 events derived from construct pM63770 and 1 event derived from Construct-2. 1 event derived from construct pM63770 was eliminated after review of the flanking sequence details provide by electronic Southern (eSouthern) data. Event GM_CSM63770 derived from construct pM63770 was selected as the commercial product based upon a final comparison of yield, efficacy and molecular characteristics between each single remaining event derived from construct pM63770 and Construct-2.

Thus, numerous rounds of testing and comparison of various constructs revealed that the transgene cassette provided as SEQ ID NO:13, Construct pM63770, when compared to events produced with all other constructs evaluated, produced events which exhibited superior efficacy against the Lepidopteran pest species Soybean podworm (SPW, Helicoverpa zea), Soybean looper (Chrysodeixis includens), Velvet bean caterpillar (Anticarsia gemmatalis), Southern armyworm (Spodoptera eridania), Black armyworm (Spodoptera cosmioides), South American podworm (Helicoverpa gelotopoeon), Sunflower looper (Rachiplusia nu), Bean shoot moth (Crocidosema aporema), Green cloverworm (Hypena scabra) and Lesser cornstalk borer (Elasmopalpus lignosellus). Soybean event GM_CSM63770 was selected from the large group of events generated using this pM63770 construct, based upon its superior characteristics with respect to efficacy and agronomic performance in comparisons to other events generated using this construct.

### Example 2

### Soybean Event GM_CSM63770 Demonstrates Resistance to the Lepidopteran Insect Pests Soybean Podworm, Soybean Looper, Velvet Bean Caterpillar, Southern Armyworm, Black Armyworm, South American Podworm, Sunflower Looper, Bean Shoot Moth, Green Cloverworm, and Lesser Cornstalk Borer

This Example describes the activity of the soybean event GM_CSM63770 against several different Lepidopteran insect pests of soybean. The insect toxin proteins Cry1A.2 and Cry1B.2, when expressed together in soybean event GM_CSM63770, provide resistance to Soybean podworm (SPW, Helicoverpa zea), Soybean looper (SBL, Chrysodeixis includens), Velvet bean caterpillar (VBC, Anticarsia gemmatalis), Southern armyworm (SAW, Spodoptera eridania), Black armyworm (BLAW, Spodoptera cosmioides), South American podworm (SAPW, Helicoverpa gelotopoeon), Sunflower looper (SFL, Rachiplusia nu), Bean shoot moth (BSM, Crocidosema aporema), Green cloverworm (GCW, Hypena scabra), and Lesser cornstalk borer (LCSB, Elasmopalpus lignosellus).

Screenhouse trials were conducted in three locations to assess resistance to Southern armyworm (SAW, Spodoptera eridania) at Velvet bean caterpillar (VBC, Anticarsia gemmatalis), and Soybean podworm (SPW, Helicoverpa zea). Soybean event GM_CSM63770 along with events derived from transformations from constructs, pM63770, Construct-2, and Construct-3 were evaluated using a randomized complete block design. Each event plot was planted in a 6-foot row with approximately 8 seed per foot. Each event had 3 reps; hence each event was represented in the screenhouse by 3 separate plots, randomly located within the screenhouse. A non-transformed event served as a negative control whose plots were also randomly assigned to locations within the screenhouse.

Infestation of SPW and VBC was accomplished using adult moths. The insects were reared to pupae in insectary adult emergence cages, maintained in climate-controlled incubators. The insects were released in the screenhouse. Approximately 1,200 to 2,000 adults were used for each release in the screenhouses. For SPW, adults were released in the screenhouse each week from the R1 to R2 stage of soybean development. With respect to VBC, adults were released in the screenhouse bi-weekly between the developmental stages of V4 to R3. Approximately 1,200 to 2,000 adults were released each time in the screenhouses. Adult moths required continuous access to a 10 percent sucrose solution for normal longevity and fecundity. Plastic food containers were filled with absorbent cotton and then the sugar solution was poured into the container to completely saturate the cotton. The sugar solution was replenished daily until adult activity subsided which was usually around 2 weeks after the final release of adults.

Direct egg infestation was used SAW since this insect does not oviposit preferentially or uniformly on soybean. Approximately 250,000 to 320,000 eggs were used for each infestation applied bi-weekly from R1 to R3 stage of development. Pieces of paper containing equal numbers of SAW eggs were attached to plants by folding the paper over a sturdy leaf petiole in the upper canopy and stapling the paper together securely. 1 paper was placed on a plant within 1 foot of the front end of the plot, a second paper was placed on a plant in the middle of the plot, and a third paper was placed on a plant within 1foot of the back end of the plot.

Defoliation for all three insects was evaluated twice after each infestation: once about 2 weeks after infestation and again at approximately four weeks after infestation. The stage of plant growth and percent defoliation were recorded after each evaluation. To determine percent defoliation, the canopy of each plot was divided into 5 zones. A trifoliate leaf was selected from each zone that exhibited damage representative of the zone from which it was selected. The percent defoliation was determined using a damage chart figure provided to each evaluator. The average percent defoliation was calculated from the average of a single, selected trifoliate leaf from each of the 5 zones. Pod production and damage for SPW was determined at maturity of the soybean plant. Five plants were randomly selected from each plot and the total number of pods and number of damaged pods on each plant were recorded. The mean number of pods and the mean percent damaged pods was determined from the sampling of the 5 plants. Table 3 below shows the mean percent defoliation assessed for soybean event GM_CSM63770 and the negative control for all three insects, SAW, VBC, and SPW. Table 4 below show the mean number of pods and the mean percent damaged pods caused by SPW.

TABLE 3
Mean percent defoliation for soybean event GM_CSM63770 and negative
control infested with SAW, VBC, and SPW in 2017 US screenhouse trials.

| | | Mean % Defoliation | | | | | | | % |
|---|---|---|---|---|---|---|---|---|---|
| | Event | R1 | R2 | R3 | R4 | Early R5 | Late R5 | Max | Reduction |
| SAW | Negative | — | 2% | 30% | 45% | 63% | 58% | 63% | — |
| | GM_CSM63770 | — | 0% | 1% | 0% | 0% | 1% | 1% | 98% |
| VBC | Negative | — | 30% | 34% | — | — | — | 34% | — |
| | GM_CSM63770 | — | 0% | 0% | — | — | — | 0% | 100% |
| SPW | Negative | 7% | 13% | — | 26% | 27% | — | 27% | — |
| | GM_CSM63770 | 1% | 3% | — | 11% | 10% | — | 11% | 60% |

TABLE 4
Mean number of pods and mean percent damaged pods for
soybean event GM_CSM63770 and negative control infested
with SPW in 2017 US screenhouse trials.

| | Mean Number | Averaged % | % Reduction |
|---|---|---|---|
| Event | Pods | Damaged | Pod Damage |
| Negative | 25 | 8% | — |
| GM_CSM63770 | 47 | 5% | 36% |

As can be seen in Table 3 above, soybean event GM_CSM63770 provided superior resistance to SAW, VBC, and SPW when compared to the negative control. In addition, as can be seen in Table 4, soybean event GM_CSM63770 demonstrated a reduced percentage of damaged pods relative to the negative control.

Screenhouse and field efficacy trials were performed. Each plot in the screenhouse comprised a row of 42 seed in a 2-meter row. Each event was represented by 3 reps randomly located within the screenhouse. Screenhouse trials were conducted against the lepidopteran insect pests, Velvet bean caterpillar (VBC, Anticarsia gemmatalis), Black armywormn (BLAW, Spodoptera cosmioides), South American podworm (SAPW, Helicoverpa gelotopoeon), and Sunflower looper (SFL, Rachiplusia nu). Approximately 500 adult moths were released into the screenhouse for each infestation. Two infestations were performed, the first at V3 stage of development and the second at R2 stage of development. Adult moths were maintained as described above using a 10 percent sugar solution to saturate absorbent cotton in a container. The percent defoliation was determined as described above. Table 5 below shows the mean percent defoliation determined for soybean event GM_CSM63770 and the negative control.

TABLE 5
Mean percent defoliation for soybean event GM_CSM63770
and negative control infested with BLAW, SAPW, SBL, and SFL.

| | | Mean % Defoliation | | | | | | % |
|---|---|---|---|---|---|---|---|---|
| | Event | V3 | V4 | V5 | R3 | R5 | Max | Reduction |
| BLAW | Negative | 9% | 28% | — | 33% | 39% | 39% | — |
| | GM_CSM63770 | 0% | 0% | — | 0% | 0% | 0% | 100% |
| SAPW | Negative | — | 16% | 29% | 38% | 48% | 48% | — |
| | GM_CSM63770 | — | 1% | 1% | 3% | 2% | 3% | 94% |
| SBL | Negative | 9% | 13% | — | — | — | 13% | — |
| | GM_CSM63770 | 0% | 0% | — | — | — | 0% | 100% |
| SFL | Negative | — | 10% | 17% | 21% | 37% | 37% | — |
| | GM_CSM63770 | — | 0% | 0% | 0% | 0% | 0% | 100% |

As can be seen in Table 5 above, soybean event GM_CSM63770 demonstrated very good resistance against BLAW, SAPW, SBL, and SFL when compared to the negative control.

Field efficacy trials were also conducted in six different locations (A-F). Each plot was comprised of 4, 10 meter rows with 25 seed per meter separated by approximately 1 meter. Each event had 3 replicate plots randomly located within the field. Sampling in the fields was performed to determine the types of Lepidopteran species and their respective percentage in each field. The Lepidopteran species noted in the field were Velvet bean caterpillar (VBC, Anticarsia gemmatalis), the looper species (SBL/SFL) Soybean looper (SBL, Chrysodeixis includens) and Sunflower looper (SFL, Rachiplusia nu), Spodoptera spp. (SPO), and South American podworm (SAPW, Helicoverpa gelotopoeon). Insect monitoring began at the beginning of V3 stage in the non-transgenic border rows with at least 10 samples evenly distributed around the border. Sampling was performed in the border rows every 7 to 10 days. Monitoring was continued until R6 stage where pod fill is complete. Defoliation was determined in a similar manner as that in the screenhouse trials. Pod damage was also performed in a similar manner as that for the screenhouse experiments. In addition, the number of larvae per meter of a planting row was also recorded. Less Lepidopteran larvae present on the transformed events when compared to the negative control plants is an indication of resistance, since resistant plants will kill the young larvae, leaving few to remain on the plant. Table 6 below shows the maximum defoliation, cumulative larvae per meter row, and the mean percent pods damaged. In addition, the percent of each Lepidopteran species is also provided for each location.

As can be seen in Table 6 below, soybean event GM_CSM63770 demonstrated Lepidopteran resistance in the field under natural infestations when compared to the negative control. The major Lepidopteran pest species in most of the fields were VBC and SBL/SFL. One location had a large percentage of SAPW and little to no VBC. From the results below, GM_CSM63770 was efficacious against VBC, SBL/SFL, and SAPW as demonstrated by the percent reduction in defoliation, lower amounts of cumulative larvae per meter row, and a reduction in pod damage.

TABLE 6
Maximum defoliation, cumulative larvae, and mean percent pod damage for soybean
event GM_CSM63770 and negative control under natural field infestations.

| | | | | % | | % | | Mean % | % |
|---|---|---|---|---|---|---|---|---|---|
| | | | Max % | Reduction | Cumulative | Reduction | Mean # | Pods | Reduction |
| Location | Insects | Event | Defoliation | Defoliation | Larvae/m Row | Larvae | Pods/Plant | Damaged | Pod Damage |
| A | VBC-64% | Negative | 13 | — | 9.3 | — | 31.3 | 3.2% | — |
| | SBL/SFL-20% | GM_CSM63770 | 2 | 85% | 1.2 | 87% | 30.3 | 0.0% | 100% |
| | SPO-13% | | | | | | | | |
| | SAPW-3% | | | | | | | | |
| B | SBL/SFL-60% | Negative | 3 | — | 6.7 | — | 37.2 | 4.2% | — |
| | SAPW-40% | GM_CSM63770 | 0 | 100% | 0.0 | 100% | 32.6 | 0.0% | 100% |
| C | VBC-67% | Negative | 16 | — | 19.4 | — | 24.0 | 0.0% | — |
| | SBL/SFL-23% | GM_CSM63770 | 3 | 81% | 3.8 | 80% | 23.3 | 0.0% | — |
| | SPO-9% | | | | | | | | |
| D | SBL/SFL-67% | Negative | 8 | — | 9.9 | — | 25.3 | 4.2% | — |
| | SAPW-22% | GM_CSM63770 | 5 | 37% | 0.0 | 100% | 30.7 | 1.1% | 74% |
| | SPO-10% | | | | | | | | |
| | VBC-1% | | | | | | | | |
| E | VBC-86% | Negative | 15 | — | 8.4 | — | 18.8 | 4.4% | — |
| | SBL/SFL-11% | GM_CSM63770 | 7 | 53% | 0.5 | 94% | 25.2 | 0.0% | 100% |
| | SPO-3% | | | | | | | | |
| F | SBL/SFL-62% | Negative | 15 | — | 6.2 | — | 23.6 | 2.3% | — |
| | VBC-23% | GM_CSM63770 | 1 | 93% | 0.3 | 95% | 18.2 | 0.0% | 100% |
| | SPO-8% | | | | | | | | |
| | SAPW-7% | | | | | | | | |

The plants in two of the locations were also examined for damage by Bean shoot moth (BSM, Crocidosema aporema). Ten plants were randomly selected from each of the three plots for each event. The average percent damage for the controls in location 1 and location 2 was 6 percent and 4.7 percent, respectively. Soybean event GM_CSM63770 demonstrated no damage at location 1 and only 0.3 percent damage at location 2.

Screenhouse efficacy trials were conducted in multiple locations against the Lepidopteran insect pest species Southern armyworm (SAW, Spodoptera eridania) Soybean looper (SBL, Chrysodeixis includens) Soybean podworm (SPW, Helicoverpa zea), and Velvet bean caterpillar (VBC, Anticarsia gemmatalis). SBL, SPW, and VBC infestations were performed using adult moths as previously described. SAW infestations were performed by egg infestation as previously described. Defoliation was determined as previously described. Plot sizes and repetitions were also as described above. Table 7 below shows the infestation frequency, the stages in which infestation occurred, the percent maximum defoliation, and the percent reduction in defoliation corresponding to event GM_CSM63770 in comparison to the negative control.

TABLE 7
Infestation frequency and stages, and percent maximum defoliation
and reduction for soybean event GM_CSM63770 and negative
control infested with SAW, SBL, SPW, and VBC.

| | | | Infestation | Infestation | % Max | % |
|---|---|---|---|---|---|---|
| Insect | Location | Event | Frequency | Growth Stages | Defoliation | Reduction |
| SAW | G | Negative | Biweekly | R1-R3 | 80% | — |
| | | GM_CSM63770 | Biweekly | R1-R3 | 0% | 100% |
| SBL | H | Negative | Biweekly | V5-R2 | 23% | — |
| | | GM_CSM63770 | Biweekly | V5-R2 | 0% | 100% |
| | | Negative | Biweekly | R3-R5 | 41% | — |
| | | GM_CSM63770 | Biweekly | R3-R5 | 0% | 100% |
| | G | Negative | Biweekly | V5-R2 | 75% | — |
| | | GM_CSM63770 | Biweekly | V5-R2 | 1% | 99% |
| SPW | I | Negative | Weekly | R1-R2 | 16% | — |
| | | GM_CSM63770 | Weekly | R1-R2 | 2% | 91% |
| VBC | I | Negative | 2x Biweekly | V5-R2 | 20% | — |
| | | GM_CSM63770 | 2x Biweekly | V5-R2 | 0% | 100% |

As can be seen in Table 7 above, soybean event GM_CSM63770 was efficacious against SAW, SBL, SPW, and VBC as demonstrated in the lower percent maximum defoliation and percent reduction in defoliation relative to the negative control. In these studies, soybean event GM_CSM63770 provided resistance to Southern armyworm (SAW, Spodoptera eridania), Soybean looper (SBL, Chrysodeixis includens), Soybean podworm (SPW, Helicoverpa zea), and Velvet bean caterpillar (VBC, Anticarsia gemmatalis).

Field efficacy trials were conducted against natural Lepidopteran pressure at five different locations (J-N). Each plot was comprised of approximately 4, 25 foot rows with approximately 8 seed per foot. Each event was represented by 4 plots randomly located within the field. Measurements of defoliation were performed in a similar manner as described above and conducted approximately every 10 days from R1 to R6 stages of development. Pod damage was assessed after the occurrence of maximum damage to the negative controls. 5 plants were randomly selected for each event to assess pod damage. Lepidopteran species population assessments were conducted during the R1 to R6 developmental stages. One or more fields were found to be infested by Velvet bean caterpillar (VBC, Anticarsia gemmatalis), Soybean looper (SBL, Chrysodeixis includens), Soybean podworm (SPW, Helicoverpa zea), Green cloverworm (GCW, Hypena scabra), Yellowstriped armyworm (YAW, Spodoptera ornithogalli), and Beet armyworm (BAW, Spodoptera exigua). The number of cumulative larvae per meter of row was also determined for each event. Table 8 below shows the percent lepidopteran species for each site along with the percent maximum and reduction in defoliation, and the cumulative number of larvae per meter row and the percent reduction of larvae.

TABLE 8
Lepidopteran species, percent maximum and reduction defoliation, cumulative
number, and percent reduction of larvae for soybean event GM_CSM63770
and negative control in fields under natural infestation.

| | | | | % | | % |
|---|---|---|---|---|---|---|
| | | | % Max | Reduction | Cumulative | Reduction |
| Location | Insects | Event | Defoliation | Defoliation | Larvae/m Row | Larvae |
| J | VBC-45% | Negative | 31% | — | 29.8 | — |
| | SBL-30% | GM_CSM63770 | 0 | 100% | 0.4 | 99% |
| | GCW-23% | | | | | |
| | YAW-2% | | | | | |
| K | SBL-85% | Negative | 13% | — | 25.9 | — |
| | VBC-1% | GM_CSM63770 | 0% | 100% | 0.2 | 99% |
| | GCW-2% | | | | | |
| | SPW-8% | | | | | |
| | BAW-2% | | | | | |
| | YAW-1% | | | | | |
| L | SBL-62% | Negative | 50% | — | 183.5 | — |
| | VBC-33% | GM_CSM63770 | 1% | 99% | 0.3 | 100% |
| | GCW-5% | | | | | |
| M | GCW-65% | Negative | 5% | — | 21.4 | — |
| | SBL-29% | GM_CSM63770 | 0% | 100% | 0.0 | 100% |
| | VBC-4% | | | | | |
| | SPW-1% | | | | | |
| | BAW-1% | | | | | |
| N | SBL-93% | Negative | 34% | — | 26.7 | — |
| | SPW-5% | GM_CSM63770 | 0% | 100% | 0.0 | 100% |
| | SMC-1% | | | | | |
| | GCW-1% | | | | | |

As can be seen in Table 8 above, based upon the percentage of the more prominent Lepidopteran species in each field, soybean event GM_CSM63770 was efficacious against Velvet bean caterpillar (VBC, Anticarsia gemmatalis), Soybean looper (SBL, Chrysodeixis includens), and Green cloverworm (GCW, Hypena scabra) as demonstrated in the percent reduction in defoliation and percent reduction of larvae for soybean event GM_CSM63770 when compared to the negative control. For example, in location D, the most predominant insect pest species were GCW and SBL and represented ninety-four percent (94%) of the total species observed in that field. The lack of larvae observed for GM_CSM63770 support that this event was highly resistant to these two species, since no living larvae could be found on the event entries, while over 21 larvae were observed per meter of row for the negative control.

Pod damage was assessed in location M, where the predominant pest species was SBL. Table 9 shows the mean percent pods damaged and the percent reduction of pod damage for soybean event GM_CSM63770.

TABLE 9
Mean percent and reduction of pod damage for soybean
event GM_CSM63770 and negative control.

| | | | | | % |
|---|---|---|---|---|---|
| | | | Mean # | Mean % | Reduction |
| | | | Pods/ | Pods | Pod |
| Location | Insects | Event | Plant | Damaged | Damage |
| M | SBL-85% | Negative | 45.1 | 2.60% | — |
| | VBC-1% | GM_CSM63770 | 40.8 | 0.50% | 80% |
| | GCW-2% | | | | |
| | SPW-8% | | | | |
| | BAW-2% | | | | |
| | YAW-1% | | | | |

As can be seen in Table 9 above, while both soybean event GM_CSM63770 and the negative control had a similar number of pods per plant, event GM_CSM63770 demonstrated an 80 percent reduction in pod damage in a field wherein most of the Lepidopteran insects were SBL. Soybean event GM_CSM63770 demonstrated resistance to SBL.

Based upon measurements of defoliation, number of larvae, and pod damage in naturally infested fields, soybean event GM_CSM63770 was efficacious against the Lepidopteran insect pests Velvet bean caterpillar (VBC, Anticarsia gemmatalis), Soybean looper (SBL, Chrysodeixis includens), and Green cloverworm (GCW, Hypena scabra).

Screenhouse and Field efficacy studies were conducted in two locations (O-P). Screenhouse trials were planted in Acevedo and Fontezuela in the Province of Buenos Aires and were conducted against the lepidopteran insect pests, Velvet bean caterpillar (VBC, Anticarsia gemmatalis), Black armyworm (BLAW, Spodoptera cosmioides), South American podworm (SAPW, Helicoverpa gelotopoeon), Soybean looper (SBL, Chrysodeixis includens), and Sunflower looper (SFL, Rachiplusia nu). Three plots similar to those described above for the 2017-2018 Argentina screenhouse trials were randomly located within the screenhouse. Each event was represented by three plots. Each screenhouse was infested with adult moths and maintained on a sugar diet as previously described. Measurements of defoliation were performed as previously described. Table 10 below shows the plant growth stages in which infestation occurred, the maximum percent and percent reduction in defoliation for each of the insect species from the two locations.

TABLE 10
Maximum percent defoliation and percent reduction of defoliation
for soybean event GM_CSM63770 and negative control.

| | | | | | % |
|---|---|---|---|---|---|
| | | Infest-1 | | Max | Defoliation |
| Insect | Location | Infest-2 | Event | Defoliation | Reduction |
| BLAW | O | LV | Negative | 23% | — |
| | | R2 | GM_CSM63770 | 0% | 100% |
| SAPW | | R1 | Negative | 33% | — |
| | | R2 | GM_CSM63770 | 2% | 94% |
| SFL | | LV | Negative | 25% | — |
| | | R2 | GM_CSM63770 | 0% | 100% |
| VBC | | LV | Negative | 23% | — |
| | | R2 | GM_CSM63770 | 0% | 100% |
| BLAW | P | LV | Negative | 27% | — |
| | | R2 | GM_CSM63770 | 1% | 96% |
| SAPW | | R1 | Negative | 53% | — |
| | | R2 | GM_CSM63770 | 2% | 96% |
| SFL | | LV | Negative | 28% | — |
| | | R2 | GM_CSM63770 | 0% | 100% |
| SBL | | R3 | Negative | 47% | — |
| | | R5 | GM_CSM63770 | 0% | 100% |

As can be seen in Table 10 above, soybean event GM_CSM63770 was efficacious against Velvet bean caterpillar (VBC, Anticarsia gemmatalis), Black armywormn (BLAW, Spodoptera cosmioides), South American podworm (SAPW, Helicoverpa gelotopoeon), Soybean looper (SBL, Chrysodeixis includens), and Sunflower looper (SFL, Rachiplusia nu).

Field trials in five locations (Q-U). Sampling in the fields was performed to determine the types of Lepidopteran species and their respective percentage in each field. The Lepidopteran species noted in the field were Velvet bean caterpillar (VBC, Anticarsia gemmatalis), the looper species (SBL/SFL) Soybean looper (SBL, Chrysodeixis includens) and Sunflower looper (SFL, Rachiplusia nu), Spodoptera spp. (SPO), and South American podworm (SAPW, Helicoverpa gelotopoeon). Each plot was comprised of 4, 8 meter rows with 25 seed per row. Each event was represented by 3 replica plots randomly located within the field. Measures of defoliation, cumulative larvae, and pod damage were as previously described. Table 11 shows the percent maximum defoliation and percent reduction in defoliation; the cumulative larvae per meter row and percent reduction in larvae; the mean percentage of pods damaged and the percent reduction in pod damage for GM_CSM63770 and negative control.

TABLE 11
Maximum defoliation, cumulative larvae, and mean percent pod damage for soybean event GM_CSM63770
and negative control under natural field infestations in Argentina during the 2017-2018 growing season.

| | | | | % | | % | | Mean % | % |
|---|---|---|---|---|---|---|---|---|---|
| | | | % Max | Reduction | Cumulative | Reduction | Mean # | pods | Reduction |
| Location | Insects | Event | defoliation | Defoliation | larvae/m row | Larvae | pods/plant | damaged | Pod Damage |
| Q | SBL/SFL-47% | Negative | 14% | — | 6.4 | — | 33.2 | 0.2% | — |
| | VBC-30% | GM_CSM63770 | 1% | 91% | 0.1 | 99% | 33.5 | 0.0% | 100% |
| | SPO-11% | | | | | | | | |
| | SAPW-13% | | | | | | | | |
| R | VBC-49% | Negative | 9% | — | 12.4 | — | 39.4 | 0.1% | — |
| | SBL/SFL-30% | GM_CSM63770 | 2% | 78% | 0.3 | 98% | 33.6 | 0.2% | 0% |
| | SPO-19% | | | | | | | | |
| | SAPW-3% | | | | | | | | |
| S | VBC-81% | Negative | 7% | — | 40.6 | — | 33.3 | 1.4% | — |
| | SBL/SFL-12% | GM_CSM63770 | 0% | 97% | 0 | 100% | 36.1 | 0.0% | 100% |
| | SPO-1% | | | | | | | | |
| | SAPW-6% | | | | | | | | |
| T | VBC-92% | Negative | 6% | — | 18.9 | — | — | — | — |
| | SBL/SFL-1% | GM_CSM63770 | 2% | 64% | 1.6 | 92% | 26.1 | 0.0% | 100% |
| | SPO-7% | | | | | | | | |
| | SAPW-0% | | | | | | | | |
| U | VBC-48% | Negative | 12% | — | 6.7 | — | 33.7 | 1.8% | — |
| | SBL/SFL-41% | GM_CSM63770 | 1% | 89% | 0.1 | 99% | 35.6 | 0.3% | 81% |
| | SPO-8% | | | | | | | | |
| | SAPW-3% | | | | | | | | |

As can be seen in Table 11 above, the largest percentage of Lepidopteran insect pests in the fields of location Q and U were VBC and SBL/SFL, whereas VBC was the predominant insect pest in the fields of location S. From the results above, soybean event GM_CSM63770 was efficacious against VBC and SBL/SFL as demonstrated by the percent reduction in defoliation, lower amounts of cumulative larvae per meter row, and a reduction in pod damage.

Soybean event GM_CSM63770 was assayed against Lesser cornstalk borer (LCSB, Elasmopalpus lignosellus) using a leaf disc assay. Eight (8) plants from soybean event GM_CSM63770 and a negative control were grown in the greenhouse. Sixteen leaf discs were harvested from each plant and used in assay with first instar neonates. Measures of mortality as well as the number of first instar neonates (mortality+L1) were taken. Table 12 below shows the average percent Mortality+L1 for GM_CSM63770 and the negative control.

TABLE 12
Average percent Mortality + L1 for soybean
event GM_CSM63770 infested with LSCB.

| Event | Mortality + L1 |
|---|---|
| Negative | 12.5% |
| GM_CSM63770 | 100.0% |

As can be seen in Table 12 above, soybean event GM_CSM63770 is very efficacious against Lesser cornstalk borer (Elasmopalpus lignosellus).

From the data presented above, soybean event GM_CSM63770 provides resistance against the Lepidopteran insect pest species Soybean podworm (Helicoverpa zea), Soybean looper (Chrysodeixis includens), Velvet bean caterpillar (Anticarsia gemmatalis), Southern armyworm (Spodoptera eridania), Black armyworm (Spodoptera cosmioides), South American podworm (Helicoverpa gelotopoeon), Sunflower looper (Rachiplusia nu), Bean shoot moth (Crocidosema aporema), Green cloverworm (Hypena scabra) and Lesser cornstalk borer (Elasmopalpus lignosellus).

### Example 3

### Soybean Event GM_CSM63770 Provides Consistent Yield and Agronomics Similar to Untransformed A3555 Soybean Plants

This Example demonstrates that transgenic soybean event GM_CSM63770 provides consistent yields and agronomics similar in the field as untransformed A3555 soybean plants.

Field trials were conducted in four separate growing seasons to evaluate agronomic traits and yield of soybean event GM_CSM63770 in comparison to the parental transformation background under field conditions. Agronomic data such as emergence, date of first flower, plant height, days to maturity, and yield (moisture corrected yield per acre) were assessed for each plot.

When compared to its transformation background A3555, soybean event GM_CSM63770 did not show any significant differences for other agronomic traits; emergence, date to flowering, and maturity, with the exception of plant height, wherein soybean event GM_CSM63770 was approximately 1 inch taller than the wildtype A3555 control, which was not considered biologically significant. Expression of the insecticidal proteins, Cry1A.2 and Cry1B.2 in soybean event GM_CSM63770 did not negatively affect the agronomic and yield performance characteristics of soybean event GM_CSM63770 when compared to the non-transgenic control.

### Example 4

### Soybean Event GM_CSM63770 Event-Specific Endpoint TAQMAN® Assays

The following Example describes methods useful in identifying the presence of GM_CSM63770 in a soybean sample. A pair of PCR primers and a probe were designed for the purpose of identifying the unique junction formed between the soybean genomic DNA and the inserted DNA of GM_CSM63770 in an event-specific endpoint TAQMAN® PCR. Examples of conditions utilized for identifying the presence of GM_CSM63770 in a soybean sample in an event-specific endpoint TAQMAN® PCR are described in Table 13 and Table 14.

The sequence of the oligonucleotide forward primer SQ13805 (SEQ ID NO:14) is identical to the nucleotide sequence corresponding to positions 13,177-13,202 of SEQ ID NO: 10. The sequence of the oligonucleotide reverse primer SQ51400 (SEQ ID NO: 15) is identical to the reverse complement of the nucleotide sequence corresponding to positions 13,280-13,310 of SEQ ID NO:10. The sequence of the oligonucleotide probe PB4832 (SEQ ID NO:16) is identical to the nucleotide sequence corresponding to positions 13,204-13,219 of SEQ ID NO:10. The primers SQ13805 (SEQ ID NO:14) and SQ51400 (SEQ ID NO:15) with probe PB4832 (SEQ ID NO:16), which may be fluorescently labeled (e.g., a 6-FAM™ fluorescent label), can be used in an endpoint TAQMAN® PCR assay to identify the presence of DNA derived from GM_CSM63770 in a sample.

In addition to SQ13805 (SEQ ID NO:14), SQ51400 (SEQ ID NO:15), and PB4832 (SEQ ID NO:16), it should be apparent to persons skilled in the art that other primers and/or probes can be designed to either amplify or hybridize to sequences within SEQ ID NO:10 which are unique to, and useful for, detecting the presence of DNA derived from GM_CSM63770 in a sample.

Following standard molecular biology laboratory practices, PCR assays for event identification were developed for detection of GM_CSM63770 in a sample. Parameters of either a standard PCR assay or a TAQMAN® PCR assay were optimized with each set of primer pairs and probes (e.g., probes labeled with a fluorescent tag such as 6-FAM™) used to detect the presence of DNA derived from GM_CSM63770 in a sample. A control for the PCR reaction includes internal control primers and an internal control probe (e.g., VIC®-labeled) specific to a region within the soybean genome that is used as an internal control, and are primers SQ549 (SEQ ID NO:17), SQ546 (SEQ ID NO:18), and VIC® labeled probe PB0004 (SEQ ID NO:19).

Generally, the parameters which were optimized for detection of GM_CSM63770 in a sample included primer and probe concentration, amount of templated DNA, and PCR amplification cycling parameters. The controls for this analysis include a positive control from soybean containing GM_CSM63770, a negative control from non-transgenic soybean, and a negative control that contains no template DNA.

TABLE 13
GM_CSM63770 event-specific endpoint TAQMAN ®
PCR reaction components.

| | | Stock | | Final | |
|---|---|---|---|---|---|
| | | Concentration | Volume | Concentration | |
| Step | Reagent | (μM) | (μl) | (μM) | Comments |
| | Reaction volume | | 5 | | |
| 1 | Master Mix | | 2.28 | | 1X final |
| | | | | | concentration |
| 2 | Event Specific | 100 | 0.05 | 0.9 | |
| | Primer SQ13805 | | | | |
| 3 | Event Specific | 100 | 0.05 | 0.9 | |
| | Primer SQ51400 | | | | |
| 4 | Event Specific | 100 | 0.01 | 0.2 | Probe is light |
| | 6FAM ™ probe | | | | sensitive |
| | PB4832 | | | | |
| 5 | Internal Control | 100 | 0.05 | 0.9 | |
| | Primer SQ549 | | | | |
| 6 | Internal Control | 100 | 0.05 | 0.9 | |
| | Primer SQ546 | | | | |
| 7 | Internal Control | 100 | 0.01 | 0.2 | Probe is light |
| | VIC ® probe | | | | sensitive |
| | PB0004 | | | | |
| 8 | Extracted DNA | | 2.5 | | Separate |
| | (template): | | | | reactions are |
| | Leaf Samples to be | | | | made for each |
| | analyzed | | | | template. |
| | Negative control | | | | |
| | (non-transgenic | | | | |
| | DNA) | | | | |
| | Negative water | | | | |
| | control (No | | | | |
| | template control) | | | | |
| | Positive | | | | |
| | Qualitative | | | | |
| | control(s) | | | | |
| | GM_CSM63770 | | | | |
| | DNA | | | | |

TABLE 14
Endpoint TAQMAN ® thermocycler conditions.

| Step No. | Number of Cycles | Settings |
|---|---|---|
| 1 | 1 | 95° C. 20 seconds |
| 2 | 35 | 95° C. 3 seconds |
| | | 60° C. 20 seconds |
| 3 | 1 | 10° C. |

### Example 5

### Assays for Determining Zygosity for Soybean Event GM_CSM63770 Using TAQMAN®

The following Example describes methods useful in identifying the zygosity of event GM_CSM63770. Pairs of PCR primers and a probe are designed for the purpose of identifying specific properties of alleles positive for the T-DNA insertion that gave rise to event GM_CSM63770 and pairs of PCR primers and a probe are designed as an internal control probe specific to a region within the soybean genome that is used as an internal control which is represented in the soybean genome as homozygous.

The pairs of PCR primers and probe specific to the GM_CSM63770 transgenic allele, described in Example 4, PCR primers SQ13805 (SEQ ID NO:14), SQ51400 (SEQ ID NO:15), and 6-FAM™ labeled probe PB4832 (SEQ ID NO:16) and the pairs of PCR primers and probe specific to the internal control, primers SQ549 (SEQ ID NO:17), SQ546 (SEQ ID NO:18), and VIC® labeled probe PB0004 (SEQ ID NO:19) are used in a real-time PCR reaction such as that described in Example 6 above.

After amplification, the cycle thresholds (Ct values) are determined for the amplicon corresponding to the GM_CSM63770 inserted allele and the single-copy, homozygous internal standard. The difference (ΔCt) between the Ct value of the single-copy, homozygous internal standard amplicon, and the Ct value of the GM_CSM63770 inserted allele amplicon are determined. With respect to zygosity, a ΔCt of around zero (0) indicates homozygosity of the inserted GM_CSM63770 T-DNA and ΔCt of around one (1) indicated heterozygosity of the inserted GM_CSM63770 T-DNA. Lack of an amplicon corresponding to the GM_CSM63770 inserted allele indicates the sample is null for the inserted GM_CSM63770 T-DNA. The Ct values in the TAQMAN® thermal amplification method will have some variability due to multiple factors such as amplification efficiency and ideal annealing temperatures. Therefore, the range of “about one (1)” is defined as a ΔCt of 0.75 to 1.25.

### Example 6

### Assays for Determining Zygosity for Soybean Event GM_CSM63770 Using TAQMAN®

The following Example describes a method useful in identifying the zygosity of event GM_CSM63770 in a soybean sample.

Pairs of PCR primers and a probe are designed for the purpose of identifying specific properties of alleles positive and negative for the T-DNA insertion that gave rise to event GM_CSM63770. Examples of conditions that may be used in an event-specific zygosity TAQMAN® PCR are provided in Tables 15 and 16. For this assay, four primers and two probes are mixed together with the sample. The DNA primer pairs used in the zygosity assay are primers SQ13805 (SEQ ID NO:14) and SQ51400 (SEQ ID NO:15); and GM_WTA3555F (SEQ ID NO:20) and SQ51400 (SEQ ID NO:15). The probes used in the zygosity assay are 6FAM™-labeled probe PB4832 (SEQ ID NO:16) and VIC®-labeled probe GM_WTA3555PB (SEQ ID NO:21). SQ13805 (SEQ ID NO:14) and SQ51400 (SEQ ID NO:15) and the 6FAM™-labeled probe PB4832 (SEQ ID NO:16) are diagnostic for, or characteristic of, soybean event GM_CSM63770 DNA. The primers GM_WTA3555F (SEQ ID NO:20) and SQ51400 (SEQ ID NO:15) and the VIC®-labeled probe GM_WTA3555PB (SEQ ID NO:21) are diagnostic when there is no copy of soybean event GM_CSM63770; i.e., they are diagnostic for, or characteristic of, the wild type allele.

When the three primers and two probes are mixed together in a PCR reaction with DNA extracted from a plant heterozygous for soybean event GM_CSM63770, there is a fluorescent signal from both the 6FAM™-labeled probe PB4832 (SEQ ID NO:16) and the VIC®-labeled probe GM_WTA3555PB (SEQ ID NO:21) which is indicative of and diagnostic for, or characteristic of, a plant heterozygous for soybean event GM_CSM63770. When the three primers and two probes are mixed together in a PCR reaction with DNA extracted from a plant homozygous for GM_CSM63770, there is a fluorescent signal from only the 6FAM™-labeled probe PB4832 (SEQ ID NO: 16) and not the VIC®-labeled probe GM_WTA3555PB (SEQ ID NO:21). When the three primers and the two probes are mixed together in a PCR reaction with DNA extracted from a plant which is null for soybean event GM_CM63770 (i.e., the wild-type), there is a fluorescent signal from only the VIC®-labeled probe GM_WTA3555PB (SEQ ID NO:21). The template DNA samples and controls for this analysis are a positive control from soybean containing GM_CSM63770 DNA (from both a known homozygous and a known heterozygous sample), a negative control from non-transgenic soybean and a negative control that contains no template DNA.

TABLE 15
GM_CSM63770 zygosity TAQMAN ® PCR.

| | | Stock | | Final | |
|---|---|---|---|---|---|
| | | Concentration | Volume | Concentration | |
| Step | Reagent | (μM) | (μl) | (μM) | Comments |
| | Reaction volume | | 5 | | |
| 1 | Master Mix | | 2.28 | | 1X final |
| | | | | | concentration |
| 2 | Event Specific | 100 | 0.05 | 0.9 | |
| | Primer SQ13805 | | | | |
| 3 | Event Specific | 100 | 0.10 | 1.8 | |
| | Primer SQ51400 | | | | |
| 4 | Event Specific | 100 | 0.01 | 0.2 | Probe is light |
| | 6FAM ™ probe | | | | sensitive |
| | PB4832 | | | | |
| 5 | Wildtype Allele | 100 | 0.05 | 0.9 | |
| | Primer | | | | |
| | GM_WTA3555F | | | | |
| 6 | Wildtype Allele | 100 | 0.01 | 0.2 | Probe is light |
| | VIC ® probe | | | | sensitive |
| | GM_WTA3555PB | | | | |
| 7 | Extracted DNA | | 2.5 | | Separate |
| | (template): | | | | reactions are |
| | Leaf Samples to be | | | | made for each |
| | analyzed | | | | template. |
| | Negative control | | | | |
| | (non-transgenic | | | | |
| | DNA) | | | | |
| | Negative water | | | | |
| | control (No template | | | | |
| | control) | | | | |
| | Positive Qualitative | | | | |
| | control(s) | | | | |
| | GM_CSM63770 | | | | |
| | DNA | | | | |

TABLE 16
Zygosity TAQMAN ® thermocycler conditions.

| Step No. | Number of Cycles | Settings |
|---|---|---|
| 1 | 1 | 95° C. 20 seconds |
| 2 | 40 | 95° C. 3 seconds |
| | | 60° C. 20 seconds |
| 3 | 1 | 10° C. |

### Example 7

### Identification of Soybean Event GM_CSM63770 in any GM_CSM63770 Breeding Event

The following Example describes how one may identify the soybean event GM_CSM63770 within progeny of any breeding activity using soybean event GM_CSM63770. For example, the soybean event GM_CSM63770 could be stacked by breeding or by site directed introgression with other events known in the art to control Lepidopteran pests such as any of the following soybean events including but not limited to MON87701, MON87751, DAS81419 (Lepidopteran resistant and herbicide tolerant). The soybean event GM_CSM63770 could also be stacked by breeding or by site directed introgression with other transgenic soybean events known in the art to provide resistance to herbicides including but not limited to A2704-12 (U.S. Patent Application Publication No. US20080320616), A5547-127 (U.S. Patent Application Publication No. US2008196127), CV127 (International Patent Application Publication No. WO2010080829), DS40278-9 (International Patent Application Publication No. WO2011022469, WO2011022470, WO2011022471), DAS44406-6 (U.S. Patent Application Publication No. US2014041083), DAS68416-4 (U.S. Patent Application Publication No. US20130296170), DAS81419-2 (U.S. Patent Application Publication No. US2013338006), DP356043-5 (U.S. Patent Application Publication No. US20100184079), FG72 (U.S. Patent Application Publication No. US2011162098), GMB151 (U.S. Patent Application Publication No. US2019345512), GTS 40-3-2, MON87708 (U.S. Patent Application Publication US, MON87751 (U.S. Patent Application No. US20140373191), MON89788, SYHT0H2 (U.S. Patent Application Publication No. US20140201860), and SYHT04R (U.S. Patent Application Publication No. US20140310835). The soybean event GM_CSM63770 could also be stacked by breeding or by site directed introgression with other transgenic soybean events known in the art to provide resistance to herbicides and modified oils, or enhanced photosynthesis, or drought tolerance including but not limited to DP305423-1 (U.S. Patent Application Publication No. US20080312082), MON87705 (U.S. Patent Application Publication No. US2014373191), MON87712 (U.S. Patent Application Publication No. US2014373191), MON87769 (U.S. Patent Application Publication No. US2011067141), and HB4 (U.S. Patent Application Publication No. US2022090114).

DNA primer pairs are used to produce an amplicon diagnostic for, or characteristic of, soybean event GM_CSM63770. An amplicon diagnostic for, or characteristic of, soybean event GM_CSM63770 comprises at least one junction sequence. The junction sequences for soybean event GM_CSM63770 are SEQ ID NO:1, SEQ ID NO:2, SEQ ID NO:3, SEQ ID NO:4, SEQ ID NO:5, and SEQ ID NO:6 ([1], [2], [3], [4], [5], and [6], respectively in FIG. 1). SEQ ID NO:1 is a 50 nucleotide sequence representing the 5′ junction regions of soybean genomic DNA and the integrated transgenic expression cassette. SEQ ID NO:1 is positioned in SEQ ID NO:10 at nucleotide position 976-1,025. SEQ ID NO:2 is a 50 nucleotide sequence representing the 3′ junction regions of soybean genomic DNA and the integrated transgenic expression cassette. SEQ ID NO:2 is positioned in SEQ ID NO:10 at nucleotide position 13,216-13,265. SEQ ID NO:3 is a 100 nucleotide sequence representing the 5′ junction regions of soybean genomic DNA and the integrated transgenic expression cassette. SEQ ID NO:3 is positioned in SEQ ID NO:10 at nucleotide position 951-1,050. SEQ ID NO:4 is a 100 nucleotide sequence representing the 3′ junction regions of soybean genomic DNA and the integrated transgenic expression cassette. SEQ ID NO:4 is positioned in SEQ ID NO:10 at nucleotide position 13,191-13,290. SEQ ID NO:5 is a 200 nucleotide sequence representing the 5′ junction regions of soybean genomic DNA and the integrated transgenic expression cassette. SEQ ID NO:5 is positioned in SEQ ID NO:10 at nucleotide position 901-1,100. SEQ ID NO:6 is a 200 nucleotide sequence representing the 3′ junction regions of soybean genomic DNA and the integrated transgenic expression cassette. SEQ ID NO:6 is positioned in SEQ ID NO:10 at nucleotide position 13,141-13,340.

Primer pairs that will produce an amplicon diagnostic for, or characteristic of, event GM_CSM63770 include primer pairs based upon the flanking sequences (SEQ ID NO:11 and SEQ ID NO:12) and the inserted T-DNA (SEQ ID NO:9). To acquire a diagnostic amplicon in which SEQ ID NO:1, or SEQ ID NO:3, or SEQ ID NO:5 is found, one would design a forward primer molecule based upon the 5′ flanking soybean genomic DNA (SEQ ID NO:11) from bases 1-1,000 and a reverse primer molecule based upon the inserted T-DNA (SEQ ID NO:9) from positions 1,001-13,240 in which the primer molecules are of sufficient length of contiguous nucleotides to specifically hybridize to SEQ ID NO:11 and SEQ ID NO:9. To acquire a diagnostic amplicon in which SEQ ID NO:2, or SEQ ID NO:4, or SEQ ID NO:6 is found, one would design a forward primer molecule based upon the inserted T-DNA (SEQ ID NO:9) from positions 1,001-13,240 and a reverse primer molecule based upon the 3′ flanking soybean genomic DNA (SEQ ID NO:12) from positions 13,241-14,240 in which the primer molecules are of sufficient length of contiguous nucleotides to specifically hybridize to SEQ ID NO:9 and SEQ ID NO:12.

For practical purposes, one should design primers which produce amplicons of a limited size range, preferably between 200 to 1000 bases. Smaller sized amplicons in general are more reliably produced in PCR reactions, allow for shorter cycle times, and can be easily separated and visualized on agarose gels or adapted for use in endpoint TAQMAN®-like assays. In addition, amplicons produced using said primer pairs can be cloned into vectors, propagated, isolated and sequenced, or can be sequenced directly with methods well established in the art. Any primer pair derived from the combinations of SEQ ID NO:11 and SEQ ID NO:9 or SEQ ID NO:12 and SEQ ID NO:9 that are useful in a DNA amplification method to produce an amplicon diagnostic for, or characteristic of, soybean event GM_CSM63770 or progeny thereof is an aspect of the present invention. Any single isolated DNA polynucleotide primer molecule comprising at least eleven (11) contiguous nucleotides of SEQ ID NO:11, SEQ ID NO:9 or SEQ ID NO:12 or their complements that is useful in a DNA amplification method to produce an amplicon diagnostic for, or characteristic of, soybean event GM_CSM63770 or progeny thereof is an aspect of the present invention.

An example of the amplification conditions for this analysis is illustrated in Tables 14 and 15. Any modification of these methods or the use of DNA primers homologous or complementary to SEQ ID NO:11 or SEQ ID NO:12, or DNA sequences of the genetic elements contained in the transgene insert (SEQ ID NO:9) of GM_CSM63770, that produce an amplicon diagnostic for, or characteristic of, soybean event GM_CSM63770 is within the art. A diagnostic amplicon comprises a DNA molecule homologous or complementary to at least one transgene/genomic junction DNA or a substantial portion thereof.

An analysis for a soybean event GM_CSM63770 plant tissue sample should include a positive tissue control from a plant that contains GM_CSM63770, a negative control from a soybean plant that does not contain GM_CSM63770 (e.g., A3555), and a negative control that contains no soybean genomic DNA. A primer pair will amplify an endogenous soybean DNA molecule and will serve as an internal control for the DNA amplification conditions. Additional primer sequences can be selected from SEQ ID NO: 11, SEQ ID NO: 12, or SEQ ID NO: 9 by those skilled in the art of DNA amplification methods. Conditions selected for the production of an amplicon by the methods shown in Table 13 and Table 14 may differ but result in an amplicon diagnostic for, or characteristic of, soybean event GM_CSM63770 DNA. The use of DNA primer sequences within or with modifications to the methods of Table 13 and Table 14 are within the scope of the invention. An amplicon produced by at least one DNA primer sequence derived from SEQ ID NO:11, SEQ ID NO:12, or SEQ ID NO:9 that is diagnostic for, or characteristic of, soybean event GM_CSM63770 is an aspect of the invention.

DNA detection kits that contain at least one DNA primer of sufficient length of contiguous nucleotides derived from SEQ ID NO:11, SEQ ID NO:12, or SEQ ID NO:9 that, when used in a DNA amplification method, produces a diagnostic amplicon for GM_CSM63770 or its progeny is an aspect of the invention. A soybean plant or seed, wherein its genome will produce an amplicon diagnostic for, or characteristic of, soybean event GM_CSM63770, when tested in a DNA amplification method is an aspect of the invention. The assay for the soybean event GM_CSM63770 amplicon can be performed by using an Applied Biosystems GeneAmp™ PCR System 9700, Stratagene Robocycler®, Eppendorf® Mastercycler® Gradient thermocycler or any other amplification system that can be used to produce an amplicon diagnostic of, or characteristic of, soybean event GM_CSM63770 as shown in Table 15.

### Example 8

### Insertion of Sequences into Soybean Event GM_CSM63770 to Facilitate Removal of the Transgene Insertion Using a Single Guide RNA

The following Example describes how one may excise the transgene insertion present in soybean event GM_CSM63770 using genomic editing techniques. Sequences useful in excision of the soybean event GM_CSM63770 transgene insertion or expression cassettes within SEQ ID NO:10 can be introduced through genomic editing using a variety of methods, particularly through the use of Clustered Regularly Interspersed Short Palindromic Repeats (CRISPR) editing systems. The CRISPR-associated protein can be selected from a Type I CRISPR-associated protein, a Type II CRISPR-associated protein, a Type III CRISPR-associated protein, a Type IV CRISPR-associated protein, Type V CRISPR-associated protein, or a Type VI CRISPR-associated protein, such as but not limited to, Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7. The CRISPR-associated protein and one or more guide RNAs (gRNA) can be introduced into a plant cell corresponding to soybean event GM_CSM63770 to target a specific sequence within the transgene insertion locus via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination thereof, at the genomic target site. One or more sequences can be inserted within the soybean event GM_CSM63770 transgene insertion locus which can allow for the excision of the transgene insertion from soybean event GM_CSM63770 or specific expression cassettes within SEQ ID NO:10.

Sequences corresponding to the 5′ and 3′ genomic flanking sequences of soybean event GM_CSM63770 (presented as SEQ ID NOs:11 and 12), the 5′ and 3′ junction regions (presented as SEQ ID NOs:1-6), the inserted T-DNA are scanned for potential originator guide RNA recognition sites (OgRRS) which comprises a protospacer adjacent motif (PAM) site operably linked to a guide RNA hybridization site. The OgRRS can be located within the flanking 5′ or 3′ genomic sequence, or within the 5′ or 3′ junction regions, or within the inserted T-DNA. The OgRRS will be determined based upon the specific CRISPR editing system chosen. For example, Cas9 recognizes a G-rich protospacer-adjacent motif (PAM) that is 3′ to its guide RNA binding site whereas Cas12a systems recognize a T-rich protospacer-adjacent motif (PAM) that is 5′ to its guide RNA binding site.

The OgRRS sequence is then used to define a cognate guide RNA recognition site (CgRRS) which is inserted into the transgene insertion locus of event GM_CSM63770 using a CRISPR editing system. The CgRRS comprises the same gRNA target sequence as the selected OgRRS. The CgRRS is inserted in a region within the transgene insertion locus of event GM_CSM63770 that is on the opposite side of the transgene insertion, relative to the OgRRS in a manner that will permit the excision of a fragment of DNA corresponding to either the entire transgene insertion of soybean event GM_CSM63770, or a fragment within the transgene insertion of soybean event GM_CSM63770 such as an expression cassette or genetic element within the transgene cassette, using a single gRNA. For example, to the extent that the OgRRS is located within the 3′ genomic flanking sequence or the 3′ junction region, then the CgRRS will be inserted within the 5′ genomic flanking sequence, or the 5′ junction region, or within the transgene insert such as between expression cassettes or genetic elements within an expression cassette. Insertion of the CgRRS on the opposite side of the transgene insertion or within the region between expression cassettes, relative to the OgRRS allows for excision of the transgene insertion or specific expression cassettes to be excised using a single gRNA. An OgRRS located between the expression cassettes of soybean event GM_CSM63770 can be used to design a CgRRS that can be inserted in either the 5′ or 3′ genomic flanking sequence to permit excision of one or the other expression cassette using a single gRNA.

Table 17 below shows exemplary OgRRS sequences located within the 5′ and 3′ genomic flanking sequences and between the two expression cassettes of soybean event GM_CSM63770 that can be used in a CRISPR editing system employing Cas12a, a Type V CRISPR-associated protein (coding sequence presented as SEQ ID NO:34; protein sequence presented as SEQ ID NO:35).

TABLE 17
Exemplary OgRRS sequences within
soybean event GM_CSM63770.

| | | SEQ | |
|---|---|---|---|
| | | ID | |
| OgRRS | Sequence | NO: | Target Site |
| OgRRS_5-1 | TTTAGAGTTTGGA | 22 | 5′ Flanking Genomic |
| | GAATAAAGTACACA | | DNA |
| OgRRS_5-2 | TTTGGCATTGGTC | 23 | 5′ Flanking Genomic |
| | GGAGTCTATGTTTC | | DNA |
| OgRRS_5-3 | TTTGGAGGGGAGAG | 24 | 5′ Flanking Genomic |
| | ATAACTACACTAA | | DNA |
| OgRRS_3-1 | TTTGGAGAGATTCT | 25 | 3′ Flanking Genomic |
| | TGGATAAATGTAT | | DNA |
| OgRRS_In-1 | TTTAGGGATAATAG | 26 | Between Expression |
| | CTGTTTGCCAATC | | Cassettes |
| OgRRS_In-2 | TTTCGCAACAGGCA | 27 | Between Expression |
| | CAGCGCTGAGGTA | | Cassettes |

Table 18 below shows gRNAs which include a poly-T transcript termination region that can be used to target the Cas12a nuclease to cut within both the OgRRS and CgRRS sequences.

TABLE 18
gRNAs useful in targeting Cas12a nuclease.

| | SEQ | |
|---|---|---|
| | ID | |
| gRNA | NO: | Sequence |
| gRNA_OgRRS_5-1 | 28 | GAATTTCTACTAAGTGT |
| | | AGATGAGTTTGGAGAAT |
| | | AAAGTACACATTTTTTT |
| gRNA_OgRRS_5-2 | 29 | GAATTTCTACTAAGTGT |
| | | AGATGCATTGGTCGGAG |
| | | TCTATGTTTCTTTTTTT |
| gRNA_OgRRS_5-3 | 30 | GAATTTCTACTAAGTGT |
| | | AGATGAGGGGAGAGATA |
| | | ACTACACTAATTTTTTT |
| gRNA_OgRRS_3-1 | 31 | GAATTTCTACTAAGTGT |
| | | AGATGAGAGATTCTTGG |
| | | ATAAATGTATTTTTTTT |
| gRNA_OgRRS_In-1 | 32 | GAATTTCTACTAAGTGT |
| | | AGATGGGATAATAGCTG |
| | | TTTGCCAATCTTTTTTT |
| gRNA_OgRRS_In-2 | 33 | GAATTTCTACTAAGTGT |
| | | AGATGCAACAGGCACA |
| | | GCGCTGAGGTATTTTTTT |

Any of the OgRRS sequences presented in Table 17 above can be used alternatively as a site to insert a CgRRS that was designed using a different OgRRS. For example, a CgRRS can be inserted into a flanking sequence to allow for the excision of the entire transgene insertion of event GM_CSM63770. To illustrate this approach, OgRRS_3-1 is selected as the OgRRS that will be used to design a corresponding CgRRS comprising DNA fragment, and OgRRS_5-3 is selected as the target site in which the CgRRS comprising DNA fragment is inserted. Using a Cas12a editing system, the OgRRS_5-3 site is targeted using the gRNA, gRNA_OgRRS_5-3 presented in Table 18 to cut within the OgRRS_5-3 site. The CgRRS comprising DNA fragment that comprises the OgRRS_3-1 target site is then inserted within the cut site that was introduced into the OgRRS_5-3 sequence. After selection of a transgenic event comprising the introduced CgRRS site, the event can be bred into another germplasm. When desired, the transgene insert of soybean event GM_CSM63770 can be excised from the plant using a Cas12a editing system and the gRNA, gRNA_OgRRS_3-1 as presented in Table 18.

Any of the OgRRS sequence presented in Table 17 that are within the 5′ and 3′ genomic flanking sequences of soybean event GM_CSM63770 can be used as a site to insert a CgRRS comprising DNA fragment, comprising an OgRRS sequence that is between expression cassettes, to permit the excision of a specific expression cassette using a single gRNA. To illustrate this approach, OgRRS_In-1 is selected as the OgRRS that will be used to design a corresponding CgRRS comprising DNA fragment, and OgRRS_5-2 is selected as the target site in which the CgRRS comprising DNA fragment is inserted. Using a Cas12a editing system, the OgRRS_5-2 site is targeted using the gRNA, gRNA_OgRRS_5-2 presented in Table 18 to cut within the OgRRS_5-2 site. The CgRRS comprising DNA fragment that comprises the OgRRS_In-1 target site is then inserted within the cut site that was introduced into the OgRRS_5-2 sequence. After selection of a transgenic event comprising the introduced CgRRS site, the event can be bred into another germplasm. When desired, the first expression cassette which expresses the Cry1A.2 toxin protein can be excised from the plant using a Cas12a editing system and the gRNA, gRNA_OgRRS_In-1 as presented in Table 18.

The CgRRS can be introduced into the transgene insertion locus through multiple methods using a CRISPR system. For example, a CRISPR system can be utilized for targeting 5′ insertion of a blunt-end double-stranded DNA fragment into a genomic target site of interest such as an OgRRS that is not the OgRRS that has been selected for the design of the CgRRS. The CRISPR-mediated endonuclease activity can introduce a double stand break (DSB) in the selected genomic target site and DNA repair, such as microhomology-driven nonhomologous end-joining DNA repair, results in insertion of the blunt-end double-stranded DNA fragment into the DSB. Blunt-end double-stranded DNA fragments can be designed with 1-10 bp of microhomology, on both the 5′ and 3′ ends of the DNA fragment, that correspond to the 5′ and 3′ flanking sequence at the cut site of the protospacer in the genomic target site.

The CRISPR system can be introduced into event GM_CSM63770 by several methods. One or more expression cassettes encoding the gRNA and/or CRISPR associated protein components of a Type I, Type II, Type III, Type IV, Type V, or Type VI CRISPR-Cas system is transiently introduced into a cell. The introduced one or more expression cassettes encoding the gRNA and/or CRISPR associated protein, along with a DNA fragment comprising the CgRRS is provided in sufficient quantity to modify the cell but does not persist after a contemplated period of time has passed or after one or more cell divisions. In such embodiments, no further steps are needed to remove or segregate the one or more expression cassettes encoding the gRNA and/or CRISPR associated protein from the modified cell.

Alternatively, an expression construct comprising one or more expression cassette for the expression of a gRNA, and an expression construct encoding a Type I, Type II, Type III, Type IV, Type V, or Type VI CRISPR associated protein is stably transformed into event GM_CSM63770 to introduce the CgRRS within the desired target locus. The gRNA will direct the nuclease to cut within the target locus which can be an OgRRS different from the selected OgRRS. The expression construct would also comprise a CgRRS DNA fragment which is flanked 5′ and 3′ with the PAM/gRNA sequence of the desired locus (i.e., an OgRRS different from the selected OgRRS) which will permit the excision of the CgRRS DNA fragment that can then be introduced into the target locus via a double strand break repair pathway.

Other Cas12a PAM/gRNA sites can be found within SEQ ID NO:10 that can be used as potential OgRRS sequences, depending upon the desired outcome after genomic editing. Table 19 below shows the coordinates of each of 418 potential OgRRS sequences within SEQ ID NO:10 and the element in which they can be found. Those indicated in bold were previously presented in Table 17.

TABLE 19
Potential OgRRS sequences within SEQ ID NO: 10 and Elements.

| | | | Coordinates | |
|---|---|---|---|---|
| PAM/ | | | within SEQ | Element within |
| gRNA | PAM | Strand | ID NO: 10 | SEQ ID NO: 10 |
| 1 | TTTA | + | 28...54 | 5′ Flanking DNA |
| 2 | TTTA | + | 79...105 | 5′ Flanking DNA |
| 3 | TTTG | + | 84...110 | 5′ Flanking DNA |
| 4 | TTTA | + | 111...137 | 5′ Flanking DNA |
| 5 | TTTA | + | 121...147 | 5′ Flanking DNA |
| 6 | TTTG | + | 129...155 | 5′ Flanking DNA |
| 7 | TTTA | + | 170...196 | 5′ Flanking DNA |
| 8 | TTTG | − | 182...156 | 5′ Flanking DNA |
| 9 | TTTA | − | 189...163 | 5′ Flanking DNA |
| 10 | TTTA | − | 203...177 | 5′ Flanking DNA |
| 11 | TTTA | − | 207...181 | 5′ Flanking DNA |
| 12 | TTTA | + | 237...263 | 5′ Flanking DNA |
| 13 | TTTG | − | 282...256 | 5′ Flanking DNA |
| 14 | TTTC | + | 369...395 | 5′ Flanking DNA |
| 15 | TTTC | + | 379...405 | 5′ Flanking DNA |
| 16 | TTTC | + | 385...411 | 5′ Flanking DNA |
| 17 | TTTG | + | 389...415 | 5′ Flanking DNA |
| 18 | TTTC | − | 403...377 | 5′ Flanking DNA |
| 19 | TTTG | − | 426...400 | 5′ Flanking DNA |
| 20 | TTTG | + | 430...456 | 5′ Flanking DNA |
| 21 | TTTC | − | 494...468 | 5′ Flanking DNA |
| 22 | TTTA | − | 503...477 | 5′ Flanking DNA |
| 23 | TTTC | − | 507...481 | 5′ Flanking DNA |
| 24 | TTTG | + | 546...572 | 5′ Flanking DNA |
| 25 | TTTG | − | 606...580 | 5′ Flanking DNA |
| 26 | TTTA | + | 632...658 | 5′ Flanking DNA |
| 27 | TTTA | − | 659...633 | 5′ Flanking DNA |
| 28 | TTTA | + | 689...715 | 5′ Flanking DNA |
| 29 | TTTA | + | 695...721 | 5′ Flanking DNA |
| 30 | TTTC | + | 765...791 | 5′ Flanking DNA |
| 31 | TTTA | + | 769...795 | 5′ Flanking DNA |
| 32 | TTTC | − | 794...768 | 5′ Flanking DNA |
| 33 | TTTA | − | 874...848 | 5′ Flanking DNA |
| 34 | TTTA | + | 890...916 | 5′ Flanking DNA |
| 35 | TTTG | − | 904...878 | 5′ Flanking DNA |
| 36 | TTTA | − | 923...897 | 5′ Flanking DNA |
| 37 | TTTC | + | 966...992 | 5′ Flanking DNA |
| 38 | TTTA | − | 990...964 | 5′ Flanking DNA |
| 39 | TTTG | − | 1021...995 | 5′ Flanking DNA/ |
| | | | | Right Border Region |
| 40 | TTTC | − | 1044...1018 | Right Border Region |
| 41 | TTTG | + | 1038...1064 | Right Border Region |
| 42 | TTTC | − | 1051...1025 | Right Border Region |
| 43 | TTTG | + | 1155...1181 | Between Right Border Region |
| | | | | and P-At.Ubq10-1:1:1 |
| 44 | TTTG | + | 1184...1210 | P-At.Ubq10-1:1:1 |
| 45 | TTTC | − | 1220...1194 | P-At.Ubq10-1:1:1 |
| 46 | TTTA | − | 1258...1232 | P-At.Ubq10-1:1:1 |
| 47 | TTTG | − | 1276...1250 | P-At.Ubq10-1:1:1 |
| 48 | TTTA | − | 1302...1276 | P-At.Ubq10-1:1:1 |
| 49 | TTTG | − | 1328...1302 | P-At.Ubq10-1:1:1 |
| 50 | TTTA | + | 1341...1367 | P-At.Ubq10-1:1:1 |
| 51 | TTTG | − | 1373...1347 | P-At.Ubq10-1:1:1 |
| 52 | TTTG | − | 1392...1366 | P-At.Ubq10-1:1:1 |
| 53 | TTTG | − | 1408...1382 | P-At.Ubq10-1:1:1 |
| 54 | TTTC | + | 1448...1474 | P-At.Ubq10-1:1:1 |
| 55 | TTTA | − | 1471...1445 | P-At.Ubq10-1:1:1 |
| 56 | TTTC | + | 1481...1507 | P-At.Ubq10-1:1:1 |
| 57 | TTTC | − | 1491...1465 | P-At.Ubq10-1:1:1 |
| 58 | TTTA | + | 1501...1527 | P-At.Ubq10-1:1:1 |
| 59 | TTTA | − | 1527...1501 | P-At.Ubq10-1:1:1 |
| 60 | TTTG | − | 1534...1508 | P-At.Ubq10-1:1:1 |
| 61 | TTTC | − | 1579...1553 | P-At.Ubq10-1:1:1 |
| 62 | TTTC | − | 1587...1561 | P-At.Ubq10-1:1:1 |
| 63 | TTTA | − | 1654...1628 | P-At.Ubq10-1:1:1 |
| 64 | TTTA | − | 1702...1676 | P-At.Ubq10-1:1:1 |
| 65 | TTTC | − | 1737...1711 | P-At.Ubq10-1:1:1 |
| 66 | TTTA | − | 1765...1739 | P-At.Ubq10-1:1:1 |
| 67 | TTTG | − | 1775...1749 | P-At.Ubq10-1:1:1 |
| 68 | TTTA | + | 1805...1831 | P-At.Ubq10-1:1:1 |
| 69 | TTTG | − | 1818...1792 | P-At.Ubq10-1:1:1 |
| 70 | TTTG | − | 1825...1799 | P-At.Ubq10-1:1:1 |
| 71 | TTTA | − | 1844...1818 | P-At.Ubq10-1:1:1 |
| 72 | TTTG | + | 1870...1896 | P-At.Ubq10-1:1:1 |
| 73 | TTTA | + | 1904...1930 | P-At.Ubq10-1:1:1 |
| 74 | TTTC | + | 1936...1962 | P-At.Ubq10-1:1:1 |
| 75 | TTTG | − | 1996...1970 | P-At.Ubq10-1:1:1 |
| 76 | TTTG | − | 2036...2010 | L-At.Ubq10-1:1:1 |
| 77 | TTTA | − | 2044...2018 | L-At.Ubq10-1:1:1 |
| 78 | TTTC | + | 2048...2074 | L-At.Ubq10-1:1:1 |
| 79 | TTTA | − | 2082...2056 | L-At.Ubq10-1:1:1 |
| 80 | TTTC | + | 2083...2109 | Between L-At.Ubq10-1:1:1 |
| | | | | and I-At.Ubq10:3 |
| 81 | TTTG | − | 2107...2081 | I-At.Ubq10:3 |
| 82 | TTTG | + | 2117...2143 | I-At.Ubq10:3 |
| 83 | TTTC | + | 2122...2148 | I-At.Ubq10:3 |
| 84 | TTTC | + | 2138...2164 | I-At.Ubq10:3 |
| 85 | TTTG | + | 2152...2178 | I-At.Ubq10:3 |
| 86 | TTTA | + | 2157...2183 | I-At.Ubq10:3 |
| 87 | TTTC | + | 2190...2216 | I-At.Ubq10:3 |
| 88 | TTTG | + | 2198...2224 | I-At.Ubq10:3 |
| 89 | TTTC | + | 2235...2261 | I-At.Ubq10:3 |
| 90 | TTTG | + | 2254...2280 | I-At.Ubq10:3 |
| 91 | TTTG | − | 2263...2237 | I-At.Ubq10:3 |
| 92 | TTTG | + | 2267...2293 | I-At.Ubq10:3 |
| 93 | TTTG | + | 2274...2300 | I-At.Ubq10:3 |
| 94 | TTTG | + | 2297...2323 | I-At.Ubq10:3 |
| 95 | TTTC | + | 2304...2330 | I-At.Ubq10:3 |
| 96 | TTTC | + | 2327...2353 | I-At.Ubq10:3 |
| 97 | TTTG | + | 2334...2360 | I-At.Ubq10:3 |
| 98 | TTTC | + | 2368...2394 | Between I-At.Ubq10:3 |
| | | | | and Cry1A.2 |
| 99 | TTTC | + | 2478...2504 | Cry1A.2 |
| 100 | TTTG | + | 2509...2535 | Cry1A.2 |
| 101 | TTTG | + | 2515...2541 | Cry1A.2 |
| 102 | TTTC | + | 2557...2583 | Cry1A.2 |
| 103 | TTTG | + | 2569...2595 | Cry1A.2 |
| 104 | TTTG | + | 2578...2604 | Cry1A.2 |
| 105 | TTTG | + | 2583...2609 | Cry1A.2 |
| 106 | TTTC | + | 2590...2616 | Cry1A.2 |
| 107 | TTTC | + | 2617...2643 | Cry1A.2 |
| 108 | TTTA | + | 2709...2735 | Cry1A.2 |
| 109 | TTTC | + | 2731...2757 | Cry1A.2 |
| 110 | TTTG | + | 2779...2805 | Cry1A.2 |
| 111 | TTTG | − | 2846...2820 | Cry1A.2 |
| 112 | TTTC | + | 2848...2874 | Cry1A.2 |
| 113 | TTTC | + | 2865...2891 | Cry1A.2 |
| 114 | TTTG | − | 2889...2863 | Cry1A.2 |
| 115 | TTTG | + | 2902...2928 | Cry1A.2 |
| 116 | TTTG | + | 2924...2950 | Cry1A.2 |
| 117 | TTTG | − | 2933...2907 | Cry1A.2 |
| 118 | TTTC | + | 2941...2967 | Cry1A.2 |
| 119 | TTTG | + | 2977...3003 | Cry1A.2 |
| 120 | TTTC | + | 2991...3017 | Cry1A.2 |
| 121 | TTTG | + | 3034...3060 | Cry1A.2 |
| 122 | TTTG | + | 3045...3071 | Cry1A.2 |
| 123 | TTTG | + | 3103...3129 | Cry1A.2 |
| 124 | TTTC | + | 3171...3197 | Cry1A.2 |
| 125 | TTTC | − | 3191...3165 | Cry1A.2 |
| 126 | TTTC | + | 3229...3255 | Cry1A.2 |
| 127 | TTTG | − | 3353...3327 | Cry1A.2 |
| 128 | TTTC | + | 3373...3399 | Cry1A.2 |
| 129 | TTTC | + | 3394...3420 | Cry1A.2 |
| 130 | TTTA | + | 3402...3428 | Cry1A.2 |
| 131 | TTTA | + | 3465...3491 | Cry1A.2 |
| 132 | TTTC | + | 3477...3503 | Cry1A.2 |
| 133 | TTTA | + | 3489...3515 | Cry1A.2 |
| 134 | TTTC | + | 3502...3528 | Cry1A.2 |
| 135 | TTTC | + | 3531...3557 | Cry1A.2 |
| 136 | TTTG | − | 3576...3550 | Cry1A.2 |
| 137 | TTTA | + | 3591...3617 | Cry1A.2 |
| 138 | TTTC | + | 3681...3707 | Cry1A.2 |
| 139 | TTTC | + | 3690...3716 | Cry1A.2 |
| 140 | TTTC | + | 3726...3752 | Cry1A.2 |
| 141 | TTTG | + | 3837...3863 | Cry1A.2 |
| 142 | TTTC | + | 3913...3939 | Cry1A.2 |
| 143 | TTTC | + | 3918...3944 | Cry1A.2 |
| 144 | TTTC | + | 4109...4135 | Cry1A.2 |
| 145 | TTTC | + | 4123...4149 | Cry1A.2 |
| 146 | TTTC | + | 4142...4168 | Cry1A.2 |
| 147 | TTTG | − | 4152...4126 | Cry1A.2 |
| 148 | TTTG | + | 4187...4213 | Cry1A.2 |
| 149 | TTTA | + | 4218...4244 | Cry1A.2 |
| 150 | TTTA | + | 4310...4336 | Cry1A.2 |
| 151 | TTTC | − | 4420...4394 | Cry1A.2 |
| 152 | TTTG | − | 4452...4426 | Cry1A.2 |
| 153 | TTTC | + | 4492...4518 | Cry1A.2 |
| 154 | TTTA | + | 4571...4597 | Cry1A.2 |
| 155 | TTTC | − | 4715...4689 | Cry1A.2 |
| 156 | TTTG | − | 4740...4714 | Cry1A.2 |
| 157 | TTTG | − | 4824...4798 | Cry1A.2 |
| 158 | TTTA | + | 4910...4936 | Cry1A.2 |
| 159 | TTTA | + | 4973...4999 | Cry1A.2 |
| 160 | TTTC | + | 5021...5047 | Cry1A.2 |
| 161 | TTTC | − | 5083...5057 | Cry1A.2 |
| 162 | TTTG | − | 5095...5069 | Cry1A.2 |
| 163 | TTTC | − | 5104...5078 | Cry1A.2 |
| 164 | TTTC | − | 5117...5091 | Cry1A.2 |
| 165 | TTTG | − | 5121...5095 | Cry1A.2 |
| 166 | TTTG | − | 5134...5108 | Cry1A.2 |
| 167 | TTTG | − | 5143...5117 | Cry1A.2 |
| 168 | TTTG | + | 5162...5188 | Cry1A.2 |
| 169 | TTTG | − | 5202...5176 | Cry1A.2 |
| 170 | TTTG | + | 5300...5326 | Cry1A.2 |
| 171 | TTTA | + | 5333...5359 | Cry1A.2 |
| 172 | TTTC | - | 5355...5329 | Cry1A.2 |
| 173 | TTTA | + | 5375...5401 | Cry1A.2 |
| 174 | TTTG | − | 5407...5381 | Cry1A.2 |
| 175 | TTTC | − | 5627...5601 | Cry1A.2 |
| 176 | TTTC | − | 5706...5680 | Cry1A.2 |
| 177 | TTTG | − | 5856...5830 | Cry1A.2 |
| 178 | TTTC | − | 5879...5853 | Cry1A.2 |
| 179 | TTTG | − | 5887...5861 | Cry1A.2 |
| 180 | TTTC | − | 5900...5874 | Cry1A.2 |
| 181 | TTTC | − | 5909...5883 | Cry1A.2 |
| 182 | TTTG | + | 5979...6005 | Between Cry1A.2 and |
| | | | | T-Mt.Zfp-1:2:1 |
| 183 | TTTG | + | 5988...6014 | T-Mt.Zfp-1:2:1 |
| 184 | TTTA | + | 6015...6041 | T-Mt.Zfp-1:2:1 |
| 185 | TTTA | + | 6020...6046 | T-Mt.Zfp-1:2:1 |
| 186 | TTTC | + | 6025...6051 | T-Mt.Zfp-1:2:1 |
| 187 | TTTG | − | 6093...6067 | T-Mt.Zfp-1:2:1 |
| 188 | TTTC | + | 6147...6173 | T-Mt.Zfp-1:2:1 |
| 189 | TTTA | + | 6155...6181 | T-Mt.Zfp-1:2:1 |
| 190 | TTTA | − | 6164...6138 | T-Mt.Zfp-1:2:1 |
| 191 | TTTA | − | 6184...6158 | T-Mt.Zfp-1:2:1 |
| 192 | TTTC | + | 6191...6217 | T-Mt.Zfp-1:2:1 |
| 193 | TTTC | − | 6225...6199 | T-Mt.Zfp-1:2:1 |
| 194 | TTTG | − | 6260...6234 | T-Mt.Zfp-1:2:1 |
| 195 | TTTA | + | 6283...6309 | T-Mt.Zfp-1:2:1 |
| 196 | TTTC | + | 6296...6322 | T-Mt.Zfp-1:2:1 |
| 197 | TTTC | + | 6309...6335 | T-Mt.Zfp-1:2:1 |
| 198 | TTTA | + | 6341...6367 | T-Mt.Zfp-1:2:1 |
| 199 | TTTC | + | 6373...6399 | T-Mt.Zfp-1:2:1 |
| 200 | TTTA | + | 6388...6414 | T-Mt.Zfp-1:2:1 |
| 201 | TTTA | + | 6406...6432 | T-Mt.Zfp-1:2:1 |
| 202 | TTTA | + | 6423...6449 | T-Mt.Zfp-1:2:1 |
| 203 | TTTA | + | 6462...6488 | T-Mt.Zfp-1:2:1 |
| 204 | TTTA | − | 6504...6478 | T-Mt.Zfp-1:2:1 |
| 205 | TTTG | − | 6521...6495 | T-Mt.Zfp-1:2:1 |
| 206 | TTTC | + | 6515...6541 | T-Mt.Zfp-1:2:1 |
| 207 | TTTA | + | 6534...6560 | T-Mt.Zfp-1:2:1 |
| 208 | TTTG | − | 6544...6518 | T-Mt.Zfp-1:2:1 |
| 209 | TTTG | − | 6550...6524 | T-Mt.Zfp-1:2:1 |
| 210 | TTTA | + | 6569...6595 | Between Cassettes |
| 211 | TTTG | + | 6586...6612 | Between Cassettes |
| 212 | TTTC | − | 6674...6648 | Between Cassettes |
| 213 | TTTC | + | 6703...6729 | P-CUCme.CipLhcb:1 |
| 214 | TTTC | + | 6804...6830 | P-CUCme.CipLhcb:1 |
| 215 | TTTG | + | 6837...6863 | P-CUCme.CipLhcb:1 |
| 216 | TTTC | + | 6882...6908 | P-CUCme.CipLhcb:1 |
| 217 | TTTC | + | 6886...6912 | P-CUCme.CipLhcb:1 |
| 218 | TTTG | − | 6911...6885 | P-CUCme.CipLhcb:1 |
| 219 | TTTA | − | 6917...6891 | P-CUCme.CipLhcb:1 |
| 220 | TTTG | + | 6968...6994 | P-CUCme.CipLhcb:1 |
| 221 | TTTA | − | 6980...6954 | P-CUCme.CipLhcb:1 |
| 222 | TTTC | + | 6973...6999 | P-CUCme.CipLhcb:1 |
| 223 | TTTA | − | 7001...6975 | P-CUCme.CipLhcb:1 |
| 224 | TTTA | + | 7003...7029 | P-CUCme.CipLhcb:1 |
| 225 | TTTC | − | 7014...6988 | P-CUCme.CipLhcb:1 |
| 226 | TTTA | − | 7019...6993 | P-CUCme.CipLhcb:1 |
| 227 | TTTA | − | 7030...7004 | P-CUCme.CipLhcb:1 |
| 228 | TTTA | − | 7035...7009 | P-CUCme.CipLhcb:1 |
| 229 | TTTC | + | 7047...7073 | P-CUCme.CipLhcb:1 |
| 230 | TTTA | + | 7057...7083 | P-CUCme.CipLhcb:1 |
| 231 | TTTG | + | 7073...7099 | P-CUCme.CipLhcb:1 |
| 232 | TTTG | + | 7088...7114 | P-CUCme.CipLhcb:1 |
| 233 | TTTG | − | 7128...7102 | P-CUCme.CipLhcb:1 |
| 234 | TTTG | − | 7146...7120 | P-CUCme.CipLhcb:1 |
| 235 | TTTC | + | 7183...7209 | P-CUCme.CipLhcb:1 |
| 236 | TTTG | + | 7188...7214 | P-CUCme.CipLhcb:1 |
| 237 | TTTA | + | 7208...7234 | P-CUCme.CipLhcb:1 |
| 238 | TTTC | + | 7234...7260 | P-CUCme.CipLhcb:1 |
| 239 | TTTG | + | 7245...7271 | P-CUCme.CipLhcb:1 |
| 240 | TTTC | − | 7268...7242 | P-CUCme.CipLhcb:1 |
| 241 | TTTG | + | 7278...7304 | P-CUCme.CipLhcb:1 |
| 242 | TTTC | + | 7298...7324 | P-CUCme.CipLhcb:1 |
| 243 | TTTA | + | 7307...7333 | P-CUCme.CipLhcb:1 |
| 244 | TTTC | − | 7329...7303 | P-CUCme.CipLhcb:1 |
| 245 | TTTC | − | 7335...7309 | P-CUCme.CipLhcb:1 |
| 246 | TTTA | − | 7357...7331 | P-CUCme.CipLhcb:1 |
| 247 | TTTA | + | 7385...7411 | P-CUCme.CipLhcb:1 |
| 248 | TTTA | + | 7393...7419 | P-CUCme.CipLhcb:1 |
| 249 | TTTA | − | 7402...7376 | P-CUCme.CipLhcb:1 |
| 250 | TTTG | − | 7420...7394 | P-CUCme.CipLhcb:1 |
| 251 | TTTG | + | 7447...7473 | P-CUCme.CipLhcb:1 |
| 252 | TTTC | + | 7475...7501 | P-CUCme.CipLhcb:1 |
| 253 | TTTG | − | 7487...7461 | P-CUCme.CipLhcb:1 |
| 254 | TTTA | + | 7504...7530 | P-CUCme.CipLhcb:1 |
| 255 | TTTA | + | 7554...7580 | P-CUCme.CipLhcb:1 |
| 256 | TTTA | − | 7582...7556 | P-CUCme.CipLhcb:1 |
| 257 | TTTA | + | 7584...7610 | P-CUCme.CipLhcb:1 |
| 258 | TTTG | + | 7588...7614 | P-CUCme.CipLhcb:1 |
| 259 | TTTA | + | 7616...7642 | P-CUCme.CipLhcb:1 |
| 260 | TTTG | + | 7629...7655 | P-CUCme.CipLhcb:1 |
| 261 | TTTA | − | 7659...7633 | P-CUCme.CipLhcb:1 |
| 262 | TTTA | + | 7708...7734 | P-CUCme.CipLhcb:1 |
| 263 | TTTA | + | 7724...7750 | P-CUCme.CipLhcb:1 |
| 264 | TTTG | − | 7744...7718 | P-CUCme.CipLhcb:1 |
| 265 | TTTC | − | 7770...7744 | P-CUCme.CipLhcb:1 |
| 266 | TTTC | − | 7789...7763 | P-CUCme.CipLhcb:1 |
| 267 | TTTC | + | 7813...7839 | P-CUCme.CipLhcb:1 |
| 268 | TTTA | + | 7867...7893 | P-CUCme.CipLhcb:1 |
| 269 | TTTC | + | 7875...7901 | P-CUCme.CipLhcb:1 |
| 270 | TTTG | + | 7880...7906 | P-CUCme.CipLhcb:1 |
| 271 | TTTA | + | 7949...7975 | P-CUCme.CipLhcb:1 |
| 272 | TTTA | + | 7959...7985 | P-CUCme.CipLhcb:1 |
| 273 | TTTG | + | 7963...7989 | P-CUCme.CipLhcb:1 |
| 274 | TTTA | − | 7983...7957 | P-CUCme.CipLhcb:1 |
| 275 | TTTA | + | 7976...8002 | P-CUCme.CipLhcb:1 |
| 276 | TTTG | + | 7985...8011 | P-CUCme.CipLhcb:1 |
| 277 | TTTC | − | 8012...7986 | P-CUCme.CipLhcb:1 |
| 278 | TTTC | + | 8035...8061 | P-CUCme.CipLhcb:1 |
| 279 | TTTA | − | 8083...8057 | P-CUCme.CipLhcb:1 |
| 280 | TTTA | − | 8095...8069 | P-CUCme.CipLhcb:1 |
| 281 | TTTA | − | 8145...8119 | P-CUCme.CipLhcb:1 |
| 282 | TTTG | + | 8147...8173 | P-CUCme.CipLhcb:1 |
| 283 | TTTA | + | 8157...8183 | P-CUCme.CipLhcb:1 |
| 284 | TTTG | − | 8198...8172 | P-CUCme.CipLhcb:1 |
| 285 | TTTA | + | 8227...8253 | P-CUCme.CipLhcb:1 |
| 286 | TTTA | − | 8246...8220 | P-CUCme.CipLhcb:1 |
| 287 | TTTA | + | 8241...8267 | P-CUCme.CipLhcb:1 |
| 288 | TTTA | + | 8249...8275 | P-CUCme.CipLhcb:1 |
| 289 | TTTG | + | 8254...8280 | P-CUCme.CipLhcb:1 |
| 290 | TTTG | − | 8281...8255 | P-CUCme.CipLhcb:1 |
| 291 | TTTA | + | 8273...8299 | P-CUCme.CipLhcb:1 |
| 292 | TTTG | − | 8286...8260 | P-CUCme.CipLhcb:1 |
| 293 | TTTG | + | 8287...8313 | P-CUCme.CipLhcb:1 |
| 294 | TTTG | − | 8312...8286 | P-CUCme.CipLhcb:1 |
| 295 | TTTA | − | 8328...8302 | P-CUCme.CipLhcb:1 |
| 296 | TTTA | + | 8330...8356 | P-CUCme.CipLhcb:1 |
| 297 | TTTC | − | 8400...8374 | P-CUCme.CipLhcb:1 |
| 298 | TTTA | − | 8409...8383 | P-CUCme.CipLhcb:1 |
| 299 | TTTA | − | 8436...8410 | P-CUCme.CipLhcb:1 |
| 300 | TTTC | − | 8449...8423 | P-CUCme.CipLhcb:1 |
| 301 | TTTG | − | 8470...8444 | P-CUCme.CipLhcb:1 |
| 302 | TTTA | + | 8477...8503 | P-CUCme.CipLhcb:1 |
| 303 | TTTG | − | 8525...8499 | P-CUCme.CipLhcb:1 |
| 304 | TTTC | + | 8527...8553 | P-CUCme.CipLhcb:1 |
| 305 | TTTC | + | 8538...8564 | P-CUCme.CipLhcb:1 |
| 306 | TTTC | + | 8637...8663 | L-CUCme.CipLhcb:1 |
| 307 | TTTC | − | 8693...8667 | Between L-CUCme.CipLhcb:1 |
| | | | | and Cry1B.2 |
| 308 | TTTG | − | 8741...8715 | Cry1B.2 |
| 309 | TTTG | − | 8908...8882 | Cry1B.2 |
| 310 | TTTC | + | 8916...8942 | Cry1B.2 |
| 311 | TTTC | − | 8971...8945 | Cry1B.2 |
| 312 | TTTG | − | 9413...9387 | Cry1B.2 |
| 313 | TTTC | + | 9627...9653 | Cry1B.2 |
| 314 | TTTC | + | 9675...9701 | Cry1B.2 |
| 315 | TTTA | + | 9841...9867 | Cry1B.2 |
| 316 | TTTA | + | 9857...9883 | Cry1B.2 |
| 317 | TTTC | + | 10100...10126 | Cry1B.2 |
| 318 | TTTG | + | 10326...10352 | Cry1B.2 |
| 319 | TTTC | + | 10501...10527 | Cry1B.2 |
| 320 | TTTC | + | 10575...10601 | Cry1B.2 |
| 321 | TTTG | − | 10788...10762 | Cry1B.2 |
| 322 | TTTG | + | 10948...10974 | Cry1B.2 |
| 323 | TTTC | − | 10998...10972 | Cry1B.2 |
| 324 | TTTA | − | 11085...11059 | Cry1B.2 |
| 325 | TTTC | − | 11301...11275 | Cry1B.2 |
| 326 | TTTC | − | 11413...11387 | Cry1B.2 |
| 327 | TTTA | − | 11555...11529 | Cry1B.2 |
| 328 | TTTC | + | 11640...11666 | Cry1B.2 |
| 329 | TTTG | − | 12134...12108 | Cry1B.2 |
| 330 | TTTC | + | 12147...12173 | Cry1B.2 |
| 331 | TTTC | − | 12178...12152 | Cry1B.2 |
| 332 | TTTC | − | 12244...12218 | Cry1B.2 |
| 333 | TTTA | + | 12253...12279 | T-Mt.Lox-1-1:2:1 |
| 334 | TTTA | + | 12257...12283 | T-Mt.Lox-1-1:2:1 |
| 335 | TTTC | + | 12278...12304 | T-Mt.Lox-1-1:2:1 |
| 336 | TTTA | − | 12287...12261 | T-Mt.Lox-1-1:2:1 |
| 337 | TTTG | − | 12308...12282 | T-Mt.Lox-1-1:2:1 |
| 338 | TTTC | + | 12370...12396 | T-Mt.Lox-1-1:2:1 |
| 339 | TTTA | + | 12381...12407 | T-Mt.Lox-1-1:2:1 |
| 340 | TTTG | + | 12385...12411 | T-Mt.Lox-1-1:2:1 |
| 341 | TTTA | − | 12407...12381 | T-Mt.Lox-1-1:2:1 |
| 342 | TTTA | + | 12402...12428 | T-Mt.Lox-1-1:2:1 |
| 343 | TTTG | + | 12410...12436 | T-Mt.Lox-1-1:2:1 |
| 344 | TTTG | + | 12417...12443 | T-Mt.Lox-1-1:2:1 |
| 345 | TTTG | − | 12430...12404 | T-Mt.Lox-1-1:2:1 |
| 346 | TTTG | − | 12448...12422 | T-Mt.Lox-1-1:2:1 |
| 347 | TTTG | + | 12467...12493 | T-Mt.Lox-1-1:2:1 |
| 348 | TTTA | + | 12475...12501 | T-Mt.Lox-1-1:2:1 |
| 349 | TTTA | + | 12523...12549 | T-Mt.Lox-1-1:2:1 |
| 350 | TTTG | + | 12527...12553 | T-Mt.Lox-1-1:2:1 |
| 351 | TTTA | − | 12537...12511 | T-Mt.Lox-1-1:2:1 |
| 352 | TTTA | + | 12554...12580 | T-Mt.Lox-1-1:2:1 |
| 353 | TTTG | + | 12604...12630 | T-Mt.Lox-1-1:2:1 |
| 354 | TTTC | − | 12647...12621 | T-Mt.Lox-1-1:2:1 |
| 355 | TTTG | − | 12703...12677 | T-Mt.Lox-1-1:2:1 |
| 356 | TTTC | + | 12695...12721 | T-Mt.Lox-1-1:2:1 |
| 357 | TTTC | + | 12714...12740 | T-Mt.Lox-1-1:2:1 |
| 358 | TTTC | + | 12718...12744 | T-Mt.Lox-1-1:2:1 |
| 359 | TTTA | − | 12818...12792 | T-Mt.Lox-1-1:2:1 |
| 360 | TTTA | + | 12813...12839 | T-Mt.Lox-1-1:2:1 |
| 361 | TTTG | − | 12854...12828 | T-Mt.Lox-1-1:2:1 |
| 362 | TTTA | − | 12873...12847 | Between T-Mt.Lox-1-1:2:1 |
| | | | | and Left Border Region |
| 363 | TTTA | + | 12868...12894 | Between T-Mt.Lox-1-1:2:1 |
| | | | | and Left Border Region |
| 364 | TTTG | + | 12888...12914 | Between T-Mt.Lox-1-1:2:1 |
| | | | | and Left Border Region |
| 365 | TTTG | − | 12906...12880 | Between T-Mt.Lox-1-1:2:1 |
| | | | | and Left Border Region |
| 366 | TTTG | + | 12987...13013 | Between T-Mt.Lox-1-1:2:1 |
| | | | | and Left Border Region |
| 367 | TTTC | − | 13014...12988 | Between T-Mt.Lox-1-1:2:1 |
| | | | | and Left Border Region |
| 368 | TTTA | − | 13018...12992 | Between T-Mt.Lox-1-1:2:1 |
| | | | | and Left Border Region |
| 369 | TTTC | + | 13021...13047 | Left Border Region |
| 370 | TTTG | + | 13038...13064 | Left Border Region |
| 371 | TTTA | + | 13042...13068 | Left Border Region |
| 372 | TTTC | + | 13050...13076 | Left Border Region |
| 373 | TTTA | − | 13062...13036 | Left Border Region |
| 374 | TTTG | + | 13078...13104 | Left Border Region |
| 375 | TTTG | − | 13094...13068 | Left Border Region |
| 376 | TTTA | + | 13086...13112 | Left Border Region |
| 377 | TTTC | + | 13101...13127 | Left Border Region |
| 378 | TTTA | + | 13107...13133 | Left Border Region |
| 379 | TTTC | + | 13172...13198 | Left Border Region |
| 380 | TTTC | + | 13215...13241 | Left Border Region |
| 381 | TTTA | + | 13234...13260 | Left Border Region/ |
| | | | | 3′ FlankingDNA |
| 382 | TTTG | − | 13266...13240 | 3′ Flanking DNA |
| 383 | TTTA | + | 13279...13305 | 3′ Flanking DNA |
| 384 | TTTC | − | 13298...13272 | 3′ Flanking DNA |
| 385 | TTTC | + | 13325...13351 | 3′ Flanking DNA |
| 386 | TTTA | + | 13335...13361 | 3′ Flanking DNA |
| 387 | TTTG | + | 13339...13365 | 3′ Flanking DNA |
| 388 | TTTC | + | 13343...13369 | 3′ Flanking DNA |
| 389 | TTTA | + | 13375...13401 | 3′ Flanking DNA |
| 390 | TTTA | − | 13513...13487 | 3′ Flanking DNA |
| 391 | TTTG | + | 13590...13616 | 3′ Flanking DNA |
| 392 | TTTG | + | 13636...13662 | 3′ Flanking DNA |
| 393 | TTTC | + | 13662...13688 | 3′ Flanking DNA |
| 394 | TTTG | − | 13715...13689 | 3′ Flanking DNA |
| 395 | TTTG | + | 13746...13720 | 3′ Flanking DNA |
| 396 | TTTG | − | 13733...13707 | 3′ Flanking DNA |
| 397 | TTTG | + | 13741...13767 | 3′ Flanking DNA |
| 398 | TTTA | + | 13796...13822 | 3′ Flanking DNA |
| 399 | TTTG | + | 13802...13828 | 3′ Flanking DNA |
| 400 | TTTG | − | 13823...13797 | 3′ Flanking DNA |
| 401 | TTTC | + | 13854...13880 | 3′ Flanking DNA |
| 402 | TTTA | − | 13865...13839 | 3′ Flanking DNA |
| 403 | TTTA | + | 13860...13886 | 3′ Flanking DNA |
| 404 | TTTA | − | 13885...13859 | 3′ Flanking DNA |
| 405 | TTTA | − | 13921...13895 | 3′ Flanking DNA |
| 406 | TTTC | − | 13933...13907 | 3′ Flanking DNA |
| 407 | TTTG | + | 13991...14017 | 3′ Flanking DNA |
| 408 | TTTC | + | 14005...14031 | 3′ Flanking DNA |
| 409 | TTTG | − | 14026...14000 | 3′ Flanking DNA |
| 410 | TTTG | + | 14027...14053 | 3′ Flanking DNA |
| 411 | TTTG | + | 14039...14065 | 3′ Flanking DNA |
| 412 | TTTA | − | 14060...14034 | 3′ Flanking DNA |
| 413 | TTTC | + | 14072...14098 | 3′ Flanking DNA |
| 414 | TTTG | + | 14097...14123 | 3′ Flanking DNA |
| 415 | TTTA | − | 14112...14086 | 3′ Flanking DNA |
| 416 | TTTG | + | 14122...14148 | 3′ Flanking DNA |
| 417 | TTTA | + | 14130...14156 | 3′ Flanking DNA |
| 418 | TTTG | + | 14184...14210 | 3′ Flanking DNA |

### Example 9

### Modification of Soybean Event GM_CSM63770 to Facilitate with Genomic Editing Techniques Using a Two Guide RNAs

This example describes the excision of all or any portion of the transgenic inserted DNA or an expression cassette within the transgenic inserted DNA defining and present in soybean event GM_CSM63770, using CRISPR editing systems comprising two guide RNAs by genomic editing methods. Excision of the event GM_CSM63770 transgenic insertion or expression cassettes within SEQ ID NO:9 or SEQ ID NO:10 can be performed through genomic editing using a variety of methods. In one embodiment, Clustered Regularly Interspersed Short Palindromic Repeats (CRISPR) editing systems comprising a CRISPR associated protein and two cognate guide RNAs may be used for targeted excision. The CRISPR-associated protein is an RNA guided nuclease and can be selected from a Type I CRISPR-associated protein, a Type II CRISPR-associated protein, a Type III CRISPR-associated protein, a Type IV CRISPR-associated protein, Type V CRISPR-associated protein, or a Type VI CRISPR-associated protein, such as but not limited to, Cas1, Cas1B, Cas2, Cas3, Cas4, Cas5, Cas6, Cas7, Cas8, Cas9 (also known as Csn1 and Csx12), Cas10, Cas 12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7. The CRISPR-associated protein and two guide RNAs (gRNA) may be introduced into a plant cell comprising the soybean event GM_CSM63770 to target a specific sequence within the transgene insertion locus. In one embodiment, the CRISPR nuclease system cleaves at two distinct guide RNA hybridization sites thereby permitting the excision of the intervening sequence. Following DNA cleavage, the genomic sequence may be repaired via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination thereof, at the genomic target site.

The guide RNAs presented in Table 18 from Example 8 are used to excise the entire transgene cassette, or alternatively, are used to remove one of the two expression cassettes in soybean event GM_CSM63770. For example, a gRNA selected from the group consisting of SEQ ID NOs:28-30 and the gRNA as presented as SEQ ID NO:31 are used to guide a Cas12a nuclease to cut within regions of the 5′ and 3′ genomic flanking sequence of soybean event GM_CSM63770 causing the excision of the entire transgene insert. Alternatively, to excise the Cry1A.2 expression cassette from soybean event GM_CSM63770 a gRNA is selected from the group consisting of SEQ ID NOs:28-30 and a gRNA is selected from the group consisting of SEQ ID NOs:32 and 33 is used to guide a Cas12a nuclease to cut within the region of the 5′ genomic flanking sequence and a region between the two transgene cassettes causing the excision of the Cry1A.2 expression cassette. Likewise, to excise the Cry1B.2 expression cassette from soybean event GM_CSM63770 a gRNA is selected from the group consisting of SEQ ID NOs:32 and 33 and the gRNA as presented as SEQ ID NO:31 is used to guide a Cas12a nuclease to cut within the region between the two transgene cassettes and with the region of the 3′ flanking genomic sequence causing excision of the Cry1B.2 expression cassette.

All publications and published patent documents cited in this specification, and which are material to the invention, are incorporated herein by reference to the same extent as if each individual publication or patent application was specifically and individually indicated to be incorporated by reference.

Having illustrated and described the principles of the present invention, it should be apparent to persons skilled in the art that the invention can be modified in arrangement and detail without departing from such principles. We claim all modifications that are within the spirit and scope of the appended claims.

1. A recombinant DNA molecule comprising a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10; and a complete complement thereof of any of the foregoing.

2. The recombinant DNA molecule of claim 1, wherein said molecule is derived from soybean event GM_CSM63770, a representative sample of seed comprising said event having been deposited as ATCC Accession No. PTA-126048.

3. A DNA molecule comprising a polynucleotide segment of sufficient length to function as a DNA probe that hybridizes specifically under stringent hybridization conditions with the recombinant DNA molecule of claim 1 in a sample, wherein detecting hybridization of said DNA molecule under said stringent hybridization conditions is diagnostic for the presence of soybean event GM_CSM63770 DNA in said sample.

4. The DNA molecule of claim 3, wherein said sample comprises a soybean plant, soybean plant cell, soybean seed, soybean plant part, soybean progeny plant, processed soybean seed, animal feed comprising soybean, soybean oil, soybean meal, soybean flour, soybean flakes, soybean bran, soybean biomass, and fuel products produced using soybean and soybean parts.

5. A pair of DNA molecules, comprising a first DNA molecule and a second DNA molecule different from the first DNA molecule, that function as DNA primers when used together in an amplification reaction with a sample containing soybean event GM_CSM63770 template DNA to produce an amplicon diagnostic for the presence of said soybean event GM_CSM63770 DNA in said sample, wherein said amplicon comprises the recombinant DNA molecule of claim 1.

6. A method of detecting the presence of a DNA segment diagnostic for soybean event GM_CSM63770 DNA in a sample, said method comprising:
a. contacting said sample with the DNA molecule of claim 3;
b. subjecting said sample and said DNA molecule to stringent hybridization conditions; and
c. detecting hybridization of said DNA molecule to said DNA in said sample, wherein said detection is diagnostic for the presence of said soybean event GM_CSM63770 DNA in said sample.

7. A method of detecting the presence of a DNA segment diagnostic for soybean event GM_CSM63770 DNA in a sample, said method comprising:
a. contacting said sample with the pair of DNA molecules of claim 5;
b. performing an amplification reaction sufficient to produce a DNA amplicon; and
c. detecting the presence of said DNA amplicon in said reaction,
wherein said DNA amplicon comprises the nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

8. A method of detecting the presence of a protein produced by a soybean plant cell comprising the recombinant DNA molecule of claim 1, wherein said protein is diagnostic for soybean event GM_CSM63770 in a sample, and wherein said recombinant DNA molecule comprises SEQ ID NO:9 or SEQ ID NO:10, said method comprising:
a. contacting said sample with a first and second monoclonal antibody, wherein the first monoclonal antibody binds specifically to Cry1A.2 and the second monoclonal antibody binds specifically to Cry1B.2;
b. incubating the protein binding assay for a sufficient amount of time to allow for binding of the monoclonal antibodies; and
c. detecting the presence of the Cry1A.2 and Cry1B.2 proteins in said assay, wherein said detection is diagnostic for the presence of said soybean event GM_CSM63770 DNA in said sample.

9. The method of claim 8, wherein the assay is performed as an Enzyme-linked Immunosorbent Assay (ELISA), a Radioimmunoassay, or a Lateral flow immunochromatographic assay.

10. A soybean plant, soybean plant part, soybean cell, or part thereof comprising soybean event GM_CSM63770 DNA characterized by the detectable presence of the recombinant DNA molecule of claim 1; wherein said soybean plant, plant part, cell, or part thereof exhibits insecticidal activity when provided in the diet of a Lepidopteran insect pest.

11. The soybean plant, soybean plant part, soybean cell, or part thereof of claim 10, wherein the Lepidopteran insect pest is selected from the group consisting of Soybean podworm (Helicoverpa zea), Soybean looper (Chrysodeixis includens), Velvet bean caterpillar (Anticarsia gemmatalis), Southern armyworm (Spodoptera eridania), Black armyworm (Spodoptera cosmioides), South American podworm (Helicoverpa gelotopoeon), Sunflower looper (Rachiplusia nu), Bean shoot moth (Crocidosema aporema), Green cloverworm (Hypena scabra), and Lesser cornstalk borer (Elasmopalpus lignosellus).

12. The soybean plant, soybean plant part, soybean cell, or part thereof of claim 10, wherein the soybean plant is further defined as progeny of any generation of a soybean plant comprising the soybean event GM_CSM63770.

13. A method for protecting a soybean plant from insect infestation, wherein said method comprises providing in the diet of a Lepidopteran insect pest an insecticidally effective amount of the soybean plant, soybean plant part, soybean cell, or part thereof of claim 10.

14. The method of claim 13, wherein said Lepidopteran insect pest is selected from the group consisting of Soybean podworm (Helicoverpa zea), Soybean looper (Chrysodeixis includens), Velvet bean caterpillar (Anticarsia gemmatalis), Southern armyworm (Spodoptera eridania), Black armyworm (Spodoptera cosmioides), South American podworm (Helicoverpa gelotopoeon), Sunflower looper (Rachiplusia nu), Bean shoot moth (Crocidosema aporema), Green cloverworm (Hypena scabra) and Lesser cornstalk borer (Elasmopalpus lignosellus).

15. A method of producing a Lepidopteran resistant soybean plant comprising:
a. breeding two different soybean plants with at least one of the two different soybean plants comprising soybean event GM_CSM63770 DNA to produce progeny;
b. confirming in said progeny the presence of the recombinant DNA molecule of claim 1; and
c. selecting said progeny comprising said recombinant DNA molecule;
wherein said progeny of step (c) are Lepidopteran resistant.

16. A soybean seed comprising a detectable amount of the recombinant DNA molecule of claim 1.

17. A nonliving soybean plant material or a commodity product comprising a detectable amount of the recombinant DNA molecule of claim 1.

18. A microorganism comprising a detectable amount of the recombinant DNA molecule of claim 1.

19. The microorganism of claim 18, wherein the microorganism is selected from the group consisting of a bacterial cell and a plant cell.

20. (canceled)

21. The commodity product of claim 19, further selected from the group consisting of whole or processed soybean seed, animal feed comprising soybean, soybean oil, soybean meal, soybean flour, soybean flakes, soybean bran, soybean biomass, and fuel products produced using soybean and soybean parts.

22. A soybean plant, soybean plant part, or soybean seed thereof comprising DNA functional as a template in a DNA amplification method to produce an amplicon diagnostic for the recombinant DNA molecule of claim 1.

23. A method of determining the zygosity of the soybean plant, soybean plant part, or soybean seed of claim 22 comprising:
a. contacting a sample comprising soybean DNA with a primer pair that is capable of producing an amplicon diagnostic for the allele corresponding to soybean event GM_CSM63770 DNA;
b. contacting said sample with a second primer pair that is capable of producing, using a thermal amplification reaction, an amplicon of an internal standard soybean genomic DNA known to be single-copy and homozygous in the soybean plant;
c. contacting said sample with a probe set which contains at least a first probe that specifically hybridizes to (or with) the allele DNA of soybean event GM_CSM63770, and a second probe that specifically hybridizes to the internal standard soybean genomic DNA known to be single-copy and homozygous in the soybean plant;
d. performing a DNA amplification reaction using real-time PCR and determining the cycle thresholds (Ct values) of the amplicon corresponding to the allele DNA of soybean event GM_CSM63770 and the single-copy, homozygous internal standard;
e. calculating the difference (ΔCt) between the Ct value of the single-copy, homozygous internal standard amplicon and the Ct value of the amplicon corresponding to the allele DNA of soybean event GM_CSM63770 sequence amplicon; and
f. determining zygosity, wherein a ΔCt of about zero (0) indicates homozygosity of the inserted T-DNA of event GM_CSM63770 and a ΔCt of about one (1) indicates heterozygosity of the inserted T-DNA of soybean event GM_CSM63770.

24. The method of claim 23, wherein the primer pairs are selected from the group consisting of SEQ ID NO:14 combined with SEQ ID NO:15, and SEQ ID NO:17 combined with SEQ ID NO:18; and wherein the probes are SEQ ID NO:16 and SEQ ID NO:19, or wherein the ΔCt of about one (1) indicating heterozygosity of the inserted T-DNA of GM CSM63770 is in the range of 0.75 to 1.25.

25. (canceled)

26. A method of determining the zygosity of the soybean plant, soybean plant part, or soybean seed of claim 22 comprising:
a. contacting a sample comprising soybean DNA with a set of primer pairs comprising at least two different primer pairs capable of producing a first amplicon diagnostic for soybean event GM_CSM63770 and a second amplicon diagnostic for native soybean genomic DNA devoid of soybean event GM_CSM63770;
b. performing a nucleic acid amplification reaction with the sample and the set of primer pairs; and
c. detecting in the nucleic acid amplification reaction the first amplicon diagnostic for soybean event GM_CSM63770 DNA, or the second amplicon diagnostic for native soybean genomic DNA devoid soybean event GM_CSM63770, wherein the presence of only the first amplicon is diagnostic of a soybean plant or soybean seed homozygous for soybean event GM_CSM63770 DNA, and the presence of both the first amplicon and the second amplicon is diagnostic of a soybean plant or soybean seed heterozygous for soybean event GM_CSM63770 DNA; or
d. contacting a sample comprising soybean DNA with a probe set which contains at least a first probe that specifically hybridizes to soybean event GM_CSM63770 DNA and at least a second probe that specifically hybridizes to soybean genomic DNA that was disrupted by insertion of the heterologous DNA of soybean event GM_CSM63770 and does not hybridize to event GM_CSM63770 DNA;
e. hybridizing the probe set with the sample under stringent hybridization conditions,
wherein detecting hybridization of only the first probe under the hybridization conditions is diagnostic for a homozygous allele of soybean event GM_CSM63770 DNA, and wherein detecting hybridization of both the first probe and the second probe under the hybridization conditions is diagnostic for a soybean plant or soybean seed heterozygous for soybean event GM_CSM63770 in said sample.

27. The method of claim 26, wherein the set of primer pairs comprises SEQ ID NO:14 combined with SEQ ID NO:15, and SEQ ID NO:20 combined with SEQ ID NO:15, or wherein the probe set comprises SEQ ID NO:16 and SEQ ID NO:21.

28. (canceled)

29. A DNA construct comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO: 9; and wherein the DNA construct comprises at the 5′ or 3′ end of said construct (i) at least 50 contiguous nucleotides of SEQ ID NO: 11 or SEQ ID NO:36; or (ii) at least 50 contiguous nucleotides of SEQ ID NO: 12 or SEQ ID NO:37.

30. The DNA construct of claim 29, wherein:
a. the DNA construct comprises at least 50 contiguous nucleotides of SEQ ID NO:11 or SEQ ID NO:36 at the 5′ end of the construct and at least 50 contiguous nucleotides of SEQ ID NO:12 or SEQ ID NO:37 at the 3′ end of the construct;
b. the construct comprises at the 5′ end of said construct one or more nucleotide sequences selected from SEQ ID NOs:38-137; or
c. the construct comprises at the 3′ end of said construct one or more nucleotide sequences selected from SEQ ID NOs:138-237.

31-32. (canceled)

33. A soybean plant, plant cell, plant part, or plant seed comprising the DNA construct of claim 29.

34. A soybean plant, plant cell, plant part, or plant seed comprising a recombinant DNA construct integrated in chromosome 19, wherein the recombinant DNA construct confers resistance to Lepidopteran insect pest species, and wherein the recombinant DNA construct is integrated in a position of said chromosome flanked by at least 50 contiguous nucleotides of SEQ ID NO:11 or SEQ ID NO:36 and 50 contiguous nucleotides of SEQ ID NO:12 or SEQ ID NO:37.

35. The soybean plant, plant cell, plant part, or plant seed of claim 34, wherein:
a. the at least 50 contiguous nucleotides in SEQ ID NO:11 or SEQ ID NO:36 comprise one or more nucleotide sequences selected from SEQ ID NOs:38-137, or
b. the at least 50 contiguous nucleotide of SEQ ID NO:12 or SEQ ID NO:37 comprise one or more nucleotide sequences selected from SEQ ID NOs:138-237.

36. (canceled)
